SEQUENCE LISTING (5 sequences)

SEQ ID NO: 1 - protein, 19 aa, Artificial Sequence
  Synthetic Peptide
  MISC_FEATURE (8)..(8): (R)-2-(7-octenyl)alanine
  MISC_FEATURE (15)..(15): (S)-2-(4-pentenyl)alanine
LAKLAKAAAKLLKAAAKAL

SEQ ID NO: 2 - protein, 19 aa, Artificial Sequence
  Synthetic Peptide
  MISC_FEATURE (1)..(1): N-Stearyl modification
  MISC_FEATURE (8)..(8): (R)-2-(7-octenyl)alanine
  MISC_FEATURE (15)..(15): (S)-2-(4-pentenyl)alanine
LAKLAKAAAKLLKAAAKAL

SEQ ID NO: 3 - protein, 19 aa, Artificial Sequence
  Synthetic Peptide
  MISC_FEATURE (8)..(8): (R)-2-(7-octenyl)alanine
  MISC_FEATURE (15)..(15): (S)-2-(7-octenyl)alanine
LAKLAKAAAKLLKAAAKAL

SEQ ID NO: 4 - protein, 19 aa, Artificial Sequence
  Synthetic Peptide
  MISC_FEATURE (2)..(2): (R)-2-(7-octenyl)alanine
  MISC_FEATURE (8)..(8): (R)-2-(7-octenyl)alanine
  MISC_FEATURE (15)..(15): (S)-2-(7-octenyl)alanine
LAKLAKAAAKLLKAAAKAL

SEQ ID NO: 5 - protein, 19 aa, Artificial Sequence
  Synthetic Peptide
  MISC_FEATURE (2)..(2): (R)-2-(7-octenyl)alanine
  MISC_FEATURE (15)..(15): (S)-2-(7-octenyl)alanine
LAKLAKAAAKLLKAAAKAL

(19) United States
(12) Patent Application Publication
(10) Pub. No.: US 2023/0227500 A1
(43) Pub. Date: Jul. 20, 2023

(54) NANOPARTICLE-LIKE DELIVERY SYSTEM

(71) Applicant: Evox Therapeutics Ltd, Oxford Oxfordshire (GB)

(72) Inventors: Samir EL ANDALOUSSI, Huddinge (SE); Taavi LEHTO, Huddinge (SE); Edvard SMITH, Stockholm (SE); Rannar SILLARD, Huddinge (SE); Kariem EZZAT, Huddinge (SE)

(21) Appl. No.: 17/777,227

(22) PCT Filed: Nov. 20, 2020
(86) PCT No.: PCT/EP2020/082986

§ 371 Date: May 16, 2022

(30) Foreign Application Priority Data
Nov. 20, 2019 (GB) 1916884.8

(51) Int. Cl. C07K 7/06; C07K 7/08; C07K 14/00; A61K 47/69

(52) U.S. Cl. CPC C07K 7/06; C07K 7/08; C07K 14/001; A61K 47/6929

(57) ABSTRACT

The present invention pertains to a new nanoparticle-like delivery system for intracellular delivery of cargo molecules such as nucleic acids, ribonucleoproteins and extracellular vesicles.

### CROSS-REFERENCE TO RELATED APPLICATIONS

This application is a national stage application filed under 35 U.S.C. 371, of International Application No. PCT/EP2020/082986, filed Nov. 20, 2020, which claims priority to, and the benefit of, GB 1916884.8, filed Nov. 20, 2019, the contents of each of which are herein incorporated by reference in their entireties.

### INCORPORATION BY REFERENCE OF SEQUENCE LISTING

The contents of the file named “EVOX-019_N01US_Sub_Seq_List_ST25.txt”, which was created on Oct. 20, 2022, and is 2,669 bytes in size are hereby incorporated by reference in their entirety.

### TECHNICAL FIELD

The present invention pertains to the field of intracellular delivery of nucleic acids, proteins, ribonucleoproteins (RNPs) and even extracellular vesicles (EVs) into cells. In particular, the invention relates to new peptides with cell penetration capability, which exhibit increased efficacy of the delivery of such cargo and low toxicity.

### BACKGROUND ART

Protein and nucleic acid-based therapeutics are extremely promising drug modalities that have the potential to treat or prevent a wide range of diseases and disorders, including those which are currently insufficiently treated or do not have any treatment at all. Similarly, therapeutics reliant on sophisticated emerging modalities, such as gene editing (for instance, using clustered regularly interspaced short palindromic repeats and associated protein 9 (CRISPR-Cas9)) and viral gene therapy, suffer from considerable delivery and/or toxicity issues, which has slowed down the impact of these inherently powerful treatment strategies. Both protein and nucleic acid-based therapies, as well as other advanced therapeutic modalities, are advantageous in that they can be designed to be extremely specific to a target therefore avoiding many side effects. However, these modalities exhibit several unsuitable properties for drug development, including poor stability in vivo, short half-life, rapid clearance, potential toxicity and immunogenicity issues, as well as an almost complete inability to penetrate the cell membrane due to their size and charge.

Nucleic acid-based therapeutics are known to suffer from poor bioavailability due to their biopolymeric properties, that is, their high molecular weight and their polyanionic nature. Messenger RNA (mRNA), in particular, is hard to deliver into cells; naked mRNA has almost no ability to naturally enter cells due to its large size and anionic charge. Lipid nanoparticles (LNPs) have been utilised for mRNA delivery with varying success, but they have very strong liver tropism and suffer from local and systemic toxicity and severe immunological side effects.

Protein-based therapeutic molecules, like antibodies, enzymes, tumour suppressors, zinc finger nucleases and transcription activator-like effector nucleases (TALENs), and RNPs such as Cas9 RNP complexes, hold enormous therapeutic potential if bioactive delivery into the intracellular space can be achieved. However, owing to their large size, these proteins and RNPs have an extremely limited ability to reach the inside of target cells, severely restricting their utility at present. Effective delivery means for protein-based therapeutic compounds are sorely lacking. In the case of enzyme replacement therapy (ERT) for various inherited metabolic disorders, delivery via the mannose-6-phosphate receptor internalisation pathway is in some cases possible, but this requires extensive protein engineering. Similarly, the delivery of antibodies across the blood-brain-barrier or into target cells has been attempted with the aid of fusion proteins to cell-penetrating peptides (CPPs) and to binders for various internalising receptors, such as the transferrin or the folate receptors.

Beyond these relatively small advantages, the delivery of protein and nucleic acid-based therapeutics into target cells has therefore become the major hurdle that must be overcome before protein therapeutics generally, and complex protein and RNP-based treatments in particular, can be used to their full potential.

EVs, such as exosomes and microvesicles, are typically nanometer-sized particles produced by most cell types. They function as the body's natural intercellular transport system for proteins, nucleic acids, peptides, lipid, and various other molecules. EVs have a number of potential therapeutic uses and are already being investigated as delivery vehicles for small molecules, proteins, mRNA and short interfering RNA (siRNA) therapeutics and for the delivery of other short nucleic acid-based drugs in various settings. EVs can be targeted to different organs using various different targeting strategies, for instance, relying on antibodies or antibody derivatives, peptides, receptors or other exosome surface engineering techniques. One inherent aspect of EVs is that they have a relatively short plasma half-life, as a result of being rapidly taken up into tissues. Interestingly, EV uptake into target cells is often more specific and less toxic than that of synthetic carriers or transfection agents, but there is clearly a need to further improve their delivery efficacy and potency.

CPPs are a promising non-viral strategy for delivery of therapeutic cargos into cells. CPPs are usually short peptides, for example, of less than 30-40 amino acids. They may be derived from proteins or chimeric sequences or may be of completely artificial, synthetic or designed origin. They are occasionally amphipathic, but almost always possess a net positive (cationic) charge at physiological pH. In some instances, CPPs are chemically stapled to increase stability and efficacy of membrane penetration. This can be useful, for instance, in the development of CPPs for disturbing bacterial membranes (Mourtada et al. (2019) Nat Biotechnol, 37(10):1186-1197) and for improving delivery efficacy. CPPs are able to penetrate biological membranes, to trigger the movement of various biomolecules across cell membranes into the cytoplasm and to improve their intracellular routing, thereby facilitating interactions with the target. Known CPPs have been shown to be able to deliver protein, nucleic acid and nanoparticle cargos into a wide variety of cell types. However, the CPPs known in the art are generally poorly tolerated, even at moderately high doses.

Therefore, there is a need in the field for new peptides that can penetrate cells, which are capable of transporting cargos of different sizes and compositions (nucleic acids, protein and vesicle cargos) and which are well tolerated at high doses.

### SUMMARY OF THE INVENTION

It is an object of the present invention to overcome the above-identified problems associated with currently known CPPs and, more generally, problems associated with inefficient intracellular delivery of sophisticated protein, nucleic acid, RNP or nanoparticle-based drug modalities. As discussed above, toxicity is a major issue for CPPs. In an attempt to lower the required dose of CPP, and thus lower the toxicity, the conventional wisdom has been to stabilise the protein backbone by using chemical stapling.

The present inventors have surprisingly demonstrated that stapling of peptides is not required when the peptides comprise the structure disclosed herein. Surprisingly, the structure of the peptides of the present invention provides them with remarkable cell penetration capacity, whilst at the same time exhibiting very low toxicity, even at high doses. The peptides of the present invention are advantageously capable of transporting a very large range of different molecules into cells.

The present invention hence pertains to a peptide comprising a first sequence A1-B1-C1-(D1)ₙ and a second sequence A2-B2-C2-(D2)ₘ, wherein:
A1 is an amino acid that has a positively charged side chain at pH 7;
B1 is an amino acid having a hydrophobic side chain;
C1 is an amino acid having a hydrophobic side chain;
D1, when present, is an amino acid having a hydrophobic side chain; and
n is 0 or 1;
wherein one of B1, C1, or D1 is substituted at the α carbon with a terminal alkenyl group or terminal alkynyl group; and
A2 is an amino acid that has a positively charged side chain at pH 7;
B2 is an amino acid having a hydrophobic side chain;
C2 is an amino acid having a hydrophobic side chain;
D2, when present, is an amino acid having a hydrophobic side chain; and
m is 0 or 1;
wherein at least one of B2, C2, or D2 is substituted at the α carbon with a terminal alkenyl group or terminal alkynyl group.

Optionally, B1 is unsubstituted. Optionally, C1 is substituted at the α carbon with a terminal alkenyl group or terminal alkynyl group. Optionally, n is 1 and D1 is unsubstituted.

Optionally B2 is unsubstituted. Optionally C2 is substituted at the α carbon with a terminal alkenyl group or terminal alkynyl group, for example a terminal alkenyl group. Optionally, m is 1 and D2 is unsubstituted.

Optionally the amino acid of the first sequence that is substituted with a terminal alkenyl or terminal alkynyl group is located at position i, and the amino acid in the second sequence that is substituted with a terminal alkenyl or terminal alkynyl group is located at i+3, 4, 5, 6, 7, 8, 9, 10, 11, 12, 13, 14, 15, or 16.

Optionally, the peptide further comprises a third sequence A3-B3-(C3)ₚ that is optionally conjugated to the C-terminus of the first sequence, wherein:
A3 is an amino acid that has a positively charged side chain at pH 7;
B3 is an amino acid having a hydrophobic side chain;
C3, when present, is an amino acid having a hydrophobic side chain; and
p is 0 or 1;
wherein B3 and C3 are independently (i) unsubstituted or (ii) substituted at the α carbon with a terminal alkenyl group or terminal alkynyl group.

Optionally, B3 is unsubstituted. Optionally, p is 1 and C3 is unsubstituted.

Optionally, the third sequence is conjugated to the C-terminus of the first sequence and the N-terminus of the second sequence.

Optionally, the peptide further comprises a fourth sequence A4-B4-(C4)_{q}, that is optionally conjugated to the N-terminus of the first sequence, wherein:
A4 is an amino acid that has a positively charged side chain at pH 7;
B4 is an amino acid having a hydrophobic side chain;
C4, when present, is an amino acid having a hydrophobic side chain; and
q is 0 or 1;
wherein B4 and C4 are independently (i) unsubstituted or (ii) substituted at the α carbon with a terminal alkenyl group or terminal alkynyl group.

Optionally, B4 is unsubstituted. Optionally, q is 1 and C4 is unsubstituted.

Optionally, the peptide further comprises a fifth sequence B5-C5-(D5)ᵣ, that is optionally conjugated to the N-terminus of the fourth sequence, wherein:
B5 is an amino acid having a hydrophobic side chain;
C5 is an amino acid having a hydrophobic side chain;
D5, when present, is an amino acid having a hydrophobic side chain; and
r is 0 or 1;
wherein B5 and C5 are independently (i) unsubstituted or (ii) substituted at the α carbon with a terminal alkenyl group or terminal alkynyl group.

Optionally, B5 is unsubstituted. Optionally, r is 0. Optionally, C5 is unsubstituted. Alternatively, C5 is substituted at the α carbon with a terminal alkenyl group or terminal alkynyl group.

Optionally, the peptide further comprises a sixth sequence A6-B6-C6-(D6)ₛ, that is optionally conjugated to the C-terminus of the second sequence, wherein:
A6 is an amino acid that has a positively charged side chain at pH 7;
B6 is an amino acid having a hydrophobic side chain;
C6 is an amino acid having a hydrophobic side chain;
D6, when present, is an amino acid having a hydrophobic side chain; and
s is 0 or 1;
wherein B6 and C6 are independently (i) unsubstituted or (ii) substituted at the α carbon with a terminal alkenyl group or terminal alkynyl group.

Optionally, B6 is unsubstituted. Optionally, C6 is unsubstituted. Optionally, s is 0.

Optionally, the peptide has the following sequence: B5-C5-(D5)ᵣ-A4-B4-(C4)_{q}-A1-B1-C1-(D1)ₙ-A3-B3-(C3)ₚ-A2-B2-C2-(D2)ₘ-A6-B6-C6-(D6)ₛ, wherein:
A1 is an amino acid that has a positively charged side chain at pH 7;
B1 is an amino acid having a hydrophobic side chain;
C1 is an amino acid having a hydrophobic side chain;
D1, when present, is an amino acid having a hydrophobic side chain; and
n is 0 or 1;
wherein one of B1, C1, or D1 is substituted at the α carbon with a terminal alkenyl group or terminal alkynyl group;
A2 is an amino acid that has a positively charged side chain at pH 7;
B2 is an amino acid having a hydrophobic side chain;
C2 is an amino acid having a hydrophobic side chain;
D2, when present, is an amino acid having a hydrophobic side chain; and
m is 0 or 1;
wherein at least one of B2, C2, or D2 is substituted at the α carbon with a terminal alkenyl group or terminal alkynyl group;
A3 is an amino acid that has a positively charged side chain at pH 7;
B3 is an amino acid having a hydrophobic side chain;
C3, when present, is an amino acid having a hydrophobic side chain; and
p is 0 or 1;
wherein B3 and C3 are independently (i) unsubstituted or (ii) substituted at the α carbon with a terminal alkenyl group or terminal alkynyl group;
A4 is an amino acid that has a positively charged side chain at pH 7;
B4 is an amino acid having a hydrophobic side chain;
C4, when present, is an amino acid having a hydrophobic side chain; and
q is 0 or 1;
wherein B4 and C4 are independently (i) unsubstituted or (ii) substituted at the α carbon with a terminal alkenyl group or terminal alkynyl group;
B5 is an amino acid having a hydrophobic side chain;
C5 is an amino acid having a hydrophobic side chain;
D5, when present, is an amino acid having a hydrophobic side chain; and
r is 0 or 1;
wherein B5 and C5 are independently (i) unsubstituted or (ii) substituted at the α carbon with a terminal alkenyl group or terminal alkynyl group, and
A6 is an amino acid that has a positively charged side chain at pH 7;
B6 is an amino acid having a hydrophobic side chain;
C6 is an amino acid having a hydrophobic side chain;
D6, when present, is an amino acid having a hydrophobic side chain; and
s is 0 or 1;
wherein B6 and C6 are independently (i) unsubstituted or (ii) substituted at the α carbon with a terminal alkenyl group or terminal alkynyl group.

Optionally, the peptide further comprises a seventh sequence [A7-B7-C7-(D7)ₜ]ᵤ, and/or an eighth sequence [A8-B8-C8-(D8)ₓ]_{y}, wherein:
A7 is an amino acid that has a positively charged side chain at pH 7;
B7 is an amino acid having a hydrophobic side chain;
C7 is an amino acid having a hydrophobic side chain;
D7, when present, is an amino acid having a hydrophobic side chain;
t is 0 or 1; and
u is 0 to 6; and
A8 is an amino acid that has a positively charged side chain at pH 7;
B8 is an amino acid having a hydrophobic side chain;
C8 is an amino acid having a hydrophobic side chain;
D8, when present, is an amino acid having a hydrophobic side chain;
x is 0 or 1; and
y is 0 to 6.

Optionally, the peptide has the following sequence: [A7-B7-C7-(D7)ₜ]ᵤ-B5-C5-(D5)ᵣ-A4-B4-(C4)_{q}-A1-B1-C1-(D1)ₙ-A3-B3-(C3)ₚ-A2-B2-C2-(D2)ₘ-A6-B6-C6(D6)ₛ-[A8-B8-C8-(D8)ₓ]_{y}, wherein:
A1 is an amino acid that has a positively charged side chain at pH 7;
B1 is an amino acid having a hydrophobic side chain;
C1 is an amino acid having a hydrophobic side chain;
D1, when present, is an amino acid having a hydrophobic side chain; and
n is 0 or 1;
wherein one of B1, C1, or D1 is substituted at the α carbon with a terminal alkenyl group or terminal alkynyl group;
A2 is an amino acid that has a positively charged side chain at pH 7;
B2 is an amino acid having a hydrophobic side chain;
C2 is an amino acid having a hydrophobic side chain;
D2, when present, is an amino acid having a hydrophobic side chain; and
m is 0 or 1;
wherein at least one of B2, C2, or D2 is substituted at the α carbon with a terminal alkenyl group or terminal alkynyl group;
A3 is an amino acid that has a positively charged side chain at pH 7;
B3 is an amino acid having a hydrophobic side chain;
C3, when present, is an amino acid having a hydrophobic side chain; and
p is 0 or 1;
wherein B3 or C3 are independently (i) unsubstituted or (ii) substituted at the α carbon with a terminal alkenyl group or terminal alkynyl group;
A4 is an amino acid that has a positively charged side chain at pH 7;
B4 is an amino acid having a hydrophobic side chain;
C4, when present, is an amino acid having a hydrophobic side chain; and
q is 0 or 1;
wherein B4 and C4 are independently (i) unsubstituted or (ii) substituted at the α carbon with a terminal alkenyl group or terminal alkynyl group;
B5 is an amino acid having a hydrophobic side chain;
C5 is an amino acid having a hydrophobic side chain;
D5, when present, is an amino acid having a hydrophobic side chain; and
r is 0 or 1;
wherein B5 and C5 are independently (i) unsubstituted or (ii) substituted at the α carbon with a terminal alkenyl group or terminal alkynyl group;
A6 is an amino acid that has a positively charged side chain at pH 7;
B6 is an amino acid having a hydrophobic side chain;
C6 is an amino acid having a hydrophobic side chain;
D6, when present, is an amino acid having a hydrophobic side chain; and
s is 0 or 1;
wherein B6 and C6 are independently unsubstituted or substituted at the α carbon with a terminal alkenyl group or terminal alkynyl group
A7 is an amino acid that has a positively charged side chain at pH 7;
B7 is an amino acid having a hydrophobic side chain;
C7 is an amino acid having a hydrophobic side chain;
D7, when present, is an amino acid having a hydrophobic side chain;
t is 0 or 1; and
u is 0 to 6; and
A8 is an amino acid that has a positively charged side chain at pH 7;
B8 is an amino acid having a hydrophobic side chain;
C8 is an amino acid having a hydrophobic side chain; and
D8, when present, is an amino acid having a hydrophobic side chain.

Optionally, each terminal alkenyl group or terminal alkynyl group comprises at least 5 carbon atoms, optionally from 5 to 20 carbon atoms, optionally from 5 to 15 carbon atoms, optionally from 5 to 11 carbon atoms, optionally from 5 to 10 carbon atoms, optionally from 5 to 8 carbon atoms.

Optionally, each alkenyl or alkynyl group is independently selected from 4-pentenyl, 5-hexenyl, 6-heptenyl, 7-octenyl, 8-nonenyl and 9-decenyl, 4-pentynyl, 5-hexynyl, 6-heptynyl, 7-octynyl, 8-nonynyl and 9-decynyl, optionally wherein each alkenyl group is independently selected from 4-pentenyl and 7-octenyl.

Optionally, the alkenyl or alkynyl groups in combination comprise at least 11 carbon atoms, optionally at least 13 carbon atoms.

Optionally, the peptide has an essentially alpha helical conformation as determined by helical wheel projection, optionally wherein the peptide has at least 90% alpha helicity, optionally at least 99% alpha helicity.

Optionally, each amino acid that has a positively charged side chain at pH 7 is individually selected from the group consisting of arginine, ornithine, histidine, lysine and analogues thereof. Each amino acid that has a positively charged side chain at pH 7 may be individually selected from the group consisting of arginine, ornithine and lysine. Optionally each amino acid that has a positively charged side chain at pH 7 is lysine or arginine.

Optionally, each amino acid having a hydrophobic side chain is individually selected from the group consisting of glycine, valine, alanine, isoleucine, leucine, proline and methionine. Optionally each amino acid having a hydrophobic side chain is individually selected from the group consisting of leucine and alanine.

The peptide optionally comprises at least 10, but no more than 100 amino acids, optionally 10-50 amino acids, 10-30 amino acids or 15-25 amino acids.

The peptide is optionally bound covalently or non-covalently to a cargo molecule.

The cargo molecule optionally is or comprises at least one biomacromolecule. The biomacromolecule may be a nanoparticle, a protein, a polypeptide, a peptide, an oligonucleotide and/or a polynucleotide, an RNP or a gene editing technology. The nanoparticle may be an EV, optionally an exosome, a microvesicle or an arrestin domain containing protein 1 (ARRDC1)-mediated microvesicle (ARRM), or a virus, optionally a lentivirus, an adenovirus, an adeno-associated virus (AAV), a retrovirus, a respiratory syncytial virus (RSV), a herpes simplex virus (HSV), an encapsulated virus, a non-encapsulated virus or a naked viral genome. The oligonucleotide and/or a polynucleotide may be a nucleic acid such as an RNA molecule, a DNA molecule or a mixmer, an antisense oligonucleotide, a splice-switching oligonucleotide, a siRNA, a short activating RNA (saRNA), a short hairpin RNA (shRNA), a microRNA (miRNA), an anti-miRNA, a plasmid DNA, a supercoiled or unsupercoiled plasmid, a mini-circle, an mRNA, a viral genome and/or viral genetic material). The RNP may be CRISPR-Cas. The gene editing technology may be a TALEN, a Zinc finger or CRISPR-Cas. In one embodiment, the cargo may be a mixture of protein, nucleic acid, virus, viral genome, antigen and/or small molecule.

According to a further aspect of the invention, there is provided a pharmaceutical composition comprising a peptide of the invention as defined above and a pharmaceutically acceptable carrier, optionally wherein the pharmaceutically acceptable carrier comprises an aqueous solution, optionally an aqueous solution comprising 4-(2-hydroxyethyl)-1-piperazineethanesulfonic acid (HEPES), wherein the pharmaceutically acceptable carrier optionally comprises a monosaccharide, disaccharide, polyvinylpyrrolidone, polyvinyl alcohol, dihydric alcohol, polyhydric alcohol (optionally sorbitol, polyethylene glycol or propylene glycol) and/or a detergent, optionally a polyoxyethylenesorbitan (Tween).

### BRIEF DESCRIPTION OF THE FIGURES

FIG. 1: (A) depicts the luciferase reporter model used to evaluate effective delivery of splice switching oligonucleotides (SSOs) by splice correction. (B) shows a bar chart which demonstrates that a range of peptides according to the invention are capable of delivering SSOs into cells at therapeutically effective levels such that they act to correct the aberrant splicing in the luciferase reporter system.

FIG. 2: shows that the peptide identified herein as Oct2815 improves the biodistribution of short antisense SSOs in vivo. (A) depicts the relative fluorescence of labelled SSO found in liver, lungs, heart, gastrointestinal (G.I.) tract, spleen, muscles and kidneys. (B) shows the fold enrichment of the delivered SSOs in the different organs. (C) shows the results of the SSO delivery to the different organs as a percentage of injected dose.

FIG. 3: shows a polyacrylamide gel following electrophoresis of RT-PCR products, which demonstrates that Oct2815 can be used to deliver charge-neutral morpholino (PMO) antisense oligonucleotides, which act to induce alternative splicing by exon skipping.

FIG. 4: demonstrates that a range of peptides according to the invention are capable of delivering functional mRNA in vivo.

FIG. 5: (A) depicts the stop-light reporter system used to assess Cas9-mediated genome editing. (B) shows the efficacy of using various peptides according to the present invention (as well as a comparison to the known CPP, Pepfect 14 (PF14)) to deliver Cas9 RNP in cells expressing the stop-light reporter system.

FIG. 6: (A) demonstrates the speed and (B) the efficacy of uptake of the Cas9 RNP into cells when complexed to Oct2815 as compared to PF14.

FIG. 7: shows that Oct2815 peptide can deliver phycoerythrin (PE)-conjugated antibodies in cells with high efficiency.

FIG. 8: shows that Oct2815 complexed with Cas9 RNP and mRNA enables delivery of both active protein and mRNA simultaneously.

FIG. 9: shows that Oct2815 can mediate intracellular delivery of EVs and significantly enhance the transfection/delivery efficacy of a variety of different types of EVs from a range of different EV cell sources.

FIG. 10: shows that Oct2815 can mediate mRNA delivery across the blood brain barrier.

FIG. 11: shows that the efficacy of Oct215 and Oct2815 are enhanced still further by the presence of different additives.

FIG. 12: shows Oct2815 is capable of delivery of siRNA.

### DETAILED DESCRIPTION OF THE INVENTION

The peptides according to the present invention are typically amphipathic, preferably have an α-helical peptide backbone and are exceptionally well-tolerated in vivo. Specifically, the peptides of the invention are tolerated in a dose of up to 30 mg/kg in mice (as illustrated herein in Example 4), which is at least 10-fold higher than the dose commonly known to be tolerated when using any CPP of the prior art in vivo.

For convenience and clarity, certain terms employed herein are collated and described below. Unless otherwise defined, all technical and scientific terms used herein have the same meaning as commonly understood by one of ordinary skill in the art.

Where features, aspects, embodiments or alternatives of the present invention are described in terms of Markush groups, a person skilled in the art will recognise that the invention is also thereby described in terms of any individual member or subgroup of members of the Markush group. The person skilled in the art will further recognise that the invention is also thereby described in terms of any combination of individual members or subgroups of members of Markush groups. Additionally, it should be noted that embodiments and features described in connection with one of the aspects and/or embodiments of the present invention also apply mutatis mutandis to all the other aspects and/or embodiments of the invention.

Throughout this disclosure, various aspects of the invention are presented in a range format. It should be understood that the description in range format is merely for convenience and brevity and should not be construed as an inflexible limitation on the scope of the invention. Accordingly, the description of a range should be considered to have specifically disclosed all the possible subranges, as well as individual numerical values, within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, each of the following terms have the meaning associated with it in this section.

The term “cell-penetrating peptide” or “CPP” or similar term shall be taken to mean a peptidyl compound, which is typically capable of translocating across a membrane system and internalising within a cell and/or within a cellular compartment.

By “peptidyl compound” is meant a peptide, or a composition the structure of which is based on a peptide such as an analogue of a peptide.

As used herein, the term “peptide” shall be taken to mean a compound other than a full-length protein that may be the expression product of a natural open-reading frame of an organism having a prokaryotic or compact eukaryote genome or a peptidyl compound of synthetic origin. The term “peptide” encompasses both oligopeptides, which have few amino acids (e.g. two to 20), and polypeptides, which have many amino acids (e.g. more than 20).

The term “amino acid”, as used herein, includes both natural and non-natural amino acids. The term “non-natural amino acid”, as used herein, refers to an organic compound that is a congener of a natural amino acid, in that it has a structure similar to a natural amino acid, so that it mimics the structure and reactivity of a natural amino acid. The non-natural amino acid can be a modified amino acid and/or an amino acid analogue that is not one of the 20 common naturally-occurring amino acids or the rare natural amino acids, selenocysteine or pyrrolysine. Non-natural amino acids can also be the D-isomer of the natural amino acids. Amino acid analogues can be natural amino acids with modified side chains or backbones. In a preferred embodiment, the analogues share backbone structures, and/or even most of the side chain structures, of one or more natural amino acids (or the D-isomer of the natural amino acids), with a difference(s) being containing one or more modified groups in the molecule. Such modification may include, without limitation, substitution of one heteroatom (such as N) for another hetero atom (such as 0 or S), addition of a group (such as an aliphatic (e.g. methyl), or hydroxyl group, etc.) or an atom (such as Cl or Br, etc.), deletion of a group (supra), substitution of a covalent bond (single bond for double bond, etc.), or combinations thereof. Amino acid analogues may include α-hydroxy acids, and α-amino acids, and can also be referred to as “modified amino acids”. Examples of suitable amino acids include, but are not limited to, alanine, allosoleucine, arginine, asparagine, aspartic acid, cysteine, glutamine, glutamic acid, glycine, histidine, isoleucine, leucine, lysine, methionine, napthylalanine, phenylalanine, proline, pyroglutamic acid, serine, threonine, tryptophan, tyrosine, valine, a derivative or combinations thereof. These, and others, are listed in Table 1 along with their abbreviations as used herein.

TABLE 1
Amino Acid Abbreviations

| Amino Acid | Abbreviations |
|---|---|
| Alanine | Ala (A) |
| Allosoleucine | Alle |
| Arginine | Arg (R) |
| Asparagine | Asn (N) |
| Aspartic acid | Asp (D) |
| Cysteine | Cys (C) |
| Cyclohexylalanine | Cha |
| 2,3-diaminopropionic acid | Dap |
| 4-fluorophenylalanine | Fpa (Σ) |
| Glutamic acid | Glu (E) |
| Glutamine | Gln (Q) |
| Glycine | Gly (G) |
| Histidine | His (H) |
| Homoproline | Pip (Θ) |
| Isoleucine | Ile (I) |
| Leucine | Leu (L) |
| Lysine | Lys (K) |
| Methionine | Met (M) |
| Napthylanine | Nal (Φ) |
| Norleucine | Nle (Ω) |
| Ornithine | Orn |
| Phenylalanine | Phe (F) |
| Phenylglycine | Phg (Ψ) |
| 4-(phosphonodifluoromethyl)phenylalanine | F₂Pmp (Λ) |
| Pipecolic acid | Pp (ϑ) |
| Proline | Pro (P) |
| Sarcosine | Sar (Ξ) |
| Selenocysteine | Sec (U) |
| Serine | Ser (S) |
| Threonine | Thr (T) |
| Tyrosine | Tyr (Y) |
| Tryptophan | Trp (W) |
| Valine | Val (V) |

The invention provides a peptide comprising a first sequence A1-B1-C1-(D1)ₙ and a second sequence A2-B2-C2-(D2)ₘ, wherein:
A1 is an amino acid that has a positively charged side chain at pH 7;
B1 is an amino acid having a hydrophobic side chain;
C1 is an amino acid having a hydrophobic side chain;
D1, when present, is an amino acid having a hydrophobic side chain; and
n is 0 or 1;
wherein one of B1, C1, or D1 is substituted at the α carbon with a terminal alkenyl group or terminal alkynyl group; and
A2 is an amino acid that has a positively charged side chain at pH 7;
B2 is an amino acid having a hydrophobic side chain;
C2 is an amino acid having a hydrophobic side chain;
D2, when present, is an amino acid having a hydrophobic side chain; and
m is 0 or 1;
wherein at least one of B2, C2, or D2 is substituted at the α carbon with a terminal alkenyl group or terminal alkynyl group.

The peptides of the invention are typically not full-length proteins that occur in nature, as defined above. For example, the peptide may be a peptide fragment of a protein, comprise at least 5 or 6 or 7 or 8 or 9 or 10 contiguous amino acid residues, and have an upper length of around 200 amino acids or 190 amino acids or 180 amino acids or 170 amino acids or 160 amino acids or 150 amino acids or 140 amino acids or 130 amino acids or 120 amino acids or 110 amino acids or 100 amino acids. Optionally the peptides have lengths in the range of 10-20 amino acids or 10-30 amino acids or 10-40 amino acids or 10-50 amino acids or 10-60 amino acids or 10-70 amino acids or 10-80 amino acids or 10-90 amino acids or 10-100 amino acids, or any length within said ranges. Particularly preferred peptides of the invention have a length in the range of about 10 to about 100 amino acids, for example, 10-95 amino acids, 11-94 amino acids or, more commonly, from about 10 to about 60 amino acids, from about 10 to about 50 amino acids or from about 10 to about 30 amino acids, for example 15-25 amino acids.

The peptides of the invention comprise two amino acids that have a positively charged side chain at pH 7 (A1, A2 as described herein).

Natural amino acids that are positively charged (i.e. that have a positively charged side chain) at pH 7 include arginine, ornithine, histidine and lysine. The peptides of the invention may include one or more of these amino acids and/or analogues thereof. Optionally, the amino acids that are positively charged at pH 7 are independently selected from arginine, ornithine and lysine. Optionally, the amino acids that are positively charged at pH 7 are independently selected from arginine and lysine. Optionally, the amino acids that are positively charged at pH 7 are each lysine.

The peptides of the invention also include four (or more) amino acids that have a hydrophobic side chain (B1, C1, B2, C2, (D1, D2 when present) as described herein).

Amino acids with a hydrophobic side chain include amino acids (or amino acid analogues) with a hydrophobicity index of 0 or greater using the method described in Kyte (1982) J Mol Biol 157(1):105-B2, for example alanine, cysteine, isoleucine, leucine, methionine, phenylalanine or valine.

The amino acid with a hydrophobic sidechain (for example, B1, C1, B2 and/or C2 as described herein) may be an amino acid or amino acid analogue with an aliphatic side chain. Aliphatic amino acids include alanine, valine, isoleucine, leucine and methionine.

Thus, each amino acid having a hydrophobic side chain may be individually selected from the group consisting of glycine, valine, alanine, isoleucine, leucine, proline and methionine, optionally wherein each amino acid having a hydrophobic side chain is individually selected from the group consisting of leucine and alanine.

In the peptides of the invention, one of B1, C1, or D1 is substituted at the α carbon with a terminal alkenyl group or terminal alkynyl group. Further, at least one of B2, C2, or D2 is substituted at the α carbon with a terminal alkenyl group or terminal alkynyl group.

“Alkenyl” or “alkenyl group” refers to a straight or branched hydrocarbon chain radical having from two to 12 carbon atoms, and having one or more carbon-carbon double bonds. Each alkenyl group is attached to the rest of the molecule by a single bond. Alkenyl groups comprising any number of carbon atoms from two to 12 are included. An alkenyl group comprising up to 12 carbon atoms is a C₂-C₁₂ alkenyl, an alkenyl group comprising up to 10 carbon atoms is a C₂-C₁₉ alkenyl, an alkenyl group comprising up to six carbon atoms is a C₂-C₆ alkenyl and an alkenyl group comprising up to five carbon atoms is a C₂-C₆ alkenyl. A C₂-C₆ alkenyl includes C₅ alkenyls, C₄ alkenyls, C₃ alkenyls, and C₂ alkenyls. A C₂-C₆ alkenyl includes all moieties described above for C₂-C₆ alkenyls but also includes C₆ alkenyls. A C₂-C₁₀ alkenyl includes all moieties described above for C₂-C₆ alkenyls and C₂-C₆ alkenyls, but also includes C₇, C₈, C₉ and C₁₀ alkenyls. Similarly, a C₂-C₁₂ alkenyl includes all of the foregoing moieties, but also includes and C₁₂ alkenyls. Non-limiting examples of C₂-C₁₂ alkenyls include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl and 11-dodecenyl. Unless specifically stated otherwise in the specification, an alkenyl group can optionally be substituted.

“Alkynyl” or “alkynyl group” refers to a straight or branched hydrocarbon chain radical having from two to 12 carbon atoms, and having one or more carbon-carbon triple bonds. Each alkynyl group is attached to the rest of the molecule by a single bond. Alkynyl groups comprising any number of carbon atoms from two to 12 are included. An alkynyl group comprising up to 12 carbon atoms is a C₂-C₁₂ alkynyl, an alkynyl comprising up to 10 carbon atoms is a C₂-C₁₀ alkynyl, an alkynyl group comprising up to six carbon atoms is a C₂-C₆ alkynyl and an alkynyl comprising up to five carbon atoms is a C₂-C₅ alkynyl. A C₂-C₅ alkynyl includes C₅ alkynyls, C₄ alkynyls, C₃ alkynyls, and C₂ alkynyls. A C₂-C₆ alkynyl includes all moieties described above for C₂-C₅ alkynyls, but also includes C₆ alkynyls. A C₂-C₁₀ alkynyl includes all moieties described above for C₂-C₅ alkynyls and C₂-C₆ alkynyls, but also includes C₇, C₈, C₉ and C₁₀ alkynyls. Similarly, a C₂-C₁₂ alkynyl includes all of the foregoing moieties, but also includes and C₁₂ alkynyls. Non-limiting examples of C₂-C₁₂ alkenyls include ethynyl, propynyl, butynyl, pentynyl and the like. Unless specifically stated otherwise in the specification, an alkynyl group can optionally be substituted.

The phrase “substituted at the α carbon with an alkenyl or alkynyl” means replacing the hydrogen bonded to the α carbon with an alkenyl or alkynyl.

The term “unsubstituted”, as used herein with reference to an amino acid, means that the amino acid is unsubstituted at the α carbon, i.e. that the hydrogen bonded to the α carbon has not been replaced by another substituent.

The term “terminal [alkenyl group or alkynyl group]”, as used herein, refers to an alkenyl or alkynyl group comprising a carbon-carbon double or triple bond, respectively, at an end of the group distal the α-carbon.

In the peptides of the invention, each terminal alkenyl group or terminal alkynyl group optionally comprises at least 5 carbon atoms, optionally from 5 to 20 carbon atoms, optionally from 5 to 15 carbon atoms, optionally from 5 to 11 carbon atoms, optionally from 5 to 10 carbon atoms, optionally from 5 to 8 carbon atoms.

Each alkenyl or alkynyl group is optionally independently selected from 4-pentenyl, 5-hexenyl, 6-heptenyl, 7-octenyl, 8-nonenyl and 9-decenyl, 4-pentynyl, 5-hexynyl, 6-heptynyl, 7-octynyl, 8-nonynyl and 9-decynyl, optionally wherein each alkenyl group is independently selected from 4-pentenyl and 7-octenyl. Optionally, the alkenyl or alkynyl groups in combination comprise at least 11 carbon atoms, optionally at least 13 carbon atoms.

The term “stapled peptide”, as used herein (with reference to known CPPs, for example), refers to a peptide that comprises at least two (typically non-adjacent) amino acids linked through a) the peptide backbone, and b) a bridge between the α-carbon atoms of each of the amino acids in question. The bridge (typically referred to as a “staple”) is typically a hydrocarbon chain, and thus the bridge is typically inert. Stapled peptides are most typically formed via the steps of i) introducing alkene or alkyne (optionally terminal alkene/alkyne) derivatives at the α-carbon atoms of the amino acids that require stapling; ii) performing a ring-closing metathesis reaction to link the two alkene/alkyne derivatives. The main reason to staple a peptide is to “lock” the peptide into a particular conformation, typically to improve the pharmacological performance of the peptide or to protect it from proteolytic degradation.

In contrast to known CPPs, the peptides of the invention do not require a staple. Surprisingly, the inventors have found that the peptides of the invention are highly effective at carrying cargo, and indeed delivering their cargo into cells, even without a staple. As such, whilst they may be stapled, the peptides of the invention are preferably unstapled. The term “unstapled peptide”, as used herein, includes peptides having alkene or alkyne (optionally terminal alkene/alkyne) derivatives at the α-carbon position, but which have not undergone the aforementioned step of ring-closing metathesis reaction, and so lack a bridge between the two amino acids in question. Likewise the term “unstapled alkenyl or alkynyl modification” refers to an alkene or alkyne group present at the α-carbon position of an amino acid which has not undergone the step of ring-closing metathesis reaction with an adjacent alkene or alkyne group.

The peptides of the invention preferably have an essentially alpha-helical conformation, which may be determined in a suitable experimental setting. Alpha-helicity can be determined using helical wheel projection using freely available tools such as EMBOSS PepWheel. The percentage of helicity can be calculated from the mean residue ellipticity at 222 nm using the method described by Low et al. (2001) J Biol Chem 276:11582-11589 and Chen et al. (1974) Biochemistry 13:3350-3359. The peptides of the invention optionally have at least 90% alpha helicity, at least 95% alpha helicity, or even at least 99% alpha helicity.

As used herein, the term “peptide” designates not only molecules in which amino acid residues (in L or D configurations or a mixture thereof) are joined by peptide (—CO—NH—) linkages, but also synthetic pseudopeptides or peptidomimetics in which the peptide bond is modified.

Peptides comprising non-natural amino acids are also within the scope of the present invention.

In the first sequence A1-B1-C1-(D1)ₙ, optionally n is 1. In the second sequence A2-B2-C2-(D2)ₘ, optionally m is 1. Optionally n is 1 and m is 1.

Optionally A1 is lysine or arginine, optionally lysine. Optionally A2 is lysine or arginine, optionally lysine. Optionally A1 and A2 are each independently selected from lysine or arginine, optionally A1 and A2 are both lysine.

Optionally B1 and/or B2 is unsubstituted.

Optionally C1 is substituted at the α carbon with a terminal alkenyl group or terminal alkynyl group. Optionally C2 is substituted at the α carbon with a terminal alkenyl group or terminal alkynyl group. Optionally C1 and C2 are both substituted at the α carbon with a terminal alkenyl group or terminal alkynyl group.

Optionally n is 1 and D1 is unsubstituted. Optionally m is 1 and D2 is unsubstituted.

Optionally the amino acid of the first sequence that is substituted with a terminal alkenyl or terminal alkynyl group is located at position i, and the amino acid in the second sequence that is substituted with a terminal alkenyl or terminal alkynyl group is located at i+3, 4, 5, 6, 7, 8, 9, 10, 11, 12, 13, 14, 15, or 16. Suitably the amino acid in the second sequence is located at i+6, 7 or 8, most preferably at i+7.

The peptides of the invention may further comprise a third sequence A3-B3-(C3)p that is optionally conjugated to the C-terminus of the first sequence, wherein:
A3 is an amino acid that has a positively charged side chain at pH 7;
B3 is an amino acid having a hydrophobic side chain;
C3, when present, is an amino acid having a hydrophobic side chain; and
p is 0 or 1;

wherein B3 and C3 are independently (i) unsubstituted or (ii) substituted at the α carbon with a terminal alkenyl group or terminal alkynyl group.

Optionally p is 1. Optionally n is 1 and p is 1. Optionally p is 1 and m is 1. Optionally n is 1, p is 1 and m is 1.

Optionally A3 is lysine or arginine, optionally lysine.

Optionally A1 and A3 are each independently selected from lysine or arginine, optionally A1 and A3 are both lysine. Optionally A2 and A3 are each independently selected from lysine or arginine, optionally A2 and A3 are both lysine.

Optionally each of A1, A2 and A3 are independently selected from lysine or arginine, optionally each of A1, A2 and A3 are lysine.

Optionally B3 is unsubstituted.

Optionally B3 is leucine, isoleucine or valine. Optionally B3 is leucine.

Optionally C3 is leucine, isoleucine or valine. Optionally C3 is leucine.

Optionally p is 1 and C3 is unsubstituted.

Optionally both B3 and C3 are each independently selected from leucine, isoleucine or valine. Optionally B3 and C3 are each leucine.

Optionally both B3 and C3 are independently selected from leucine, isoleucine or valine (optionally leucine) and A1 is lysine or arginine (optionally lysine).

Optionally both B3 and C3 are independently selected from leucine, isoleucine or valine (optionally leucine) and A2 is lysine or arginine (optionally lysine).

Optionally both B3 and C3 are independently selected from leucine, isoleucine or valine (optionally leucine) and A3 is lysine or arginine (optionally lysine).

Optionally both B3 and C3 are independently selected from leucine, isoleucine or valine (optionally leucine) and both A1 and A2 are independently selected from lysine or arginine (optionally lysine).

Optionally both B3 and C3 are independently selected from leucine, isoleucine or valine (optionally leucine) and both A1 and A3 are independently selected from lysine or arginine (optionally lysine).

Optionally both B3 and C3 are independently selected from leucine, isoleucine or valine (optionally leucine) and both A2 and A3 are independently selected from lysine or arginine (optionally lysine).

Optionally both B3 and C3 are independently selected from leucine, isoleucine or valine (optionally leucine) and A1, A2 and A3 are each independently selected from lysine or arginine (optionally lysine).

Optionally both B3 and C3 are independently selected from leucine, isoleucine or valine (optionally leucine) and each of A1, A2, A3 is independently selected from lysine or arginine (optionally lysine) and n is 1.

Optionally both B3 and C3 are independently selected from leucine, isoleucine or valine (optionally leucine) and each of A1, A2, A3 independently selected from lysine or arginine (optionally lysine) and p is 1.

Optionally both B3 and C3 are independently selected from leucine, isoleucine or valine (optionally leucine) and each of A1, A2, A3 is independently selected from lysine or arginine (optionally lysine) and m is 1.

Optionally both B3 and C3 are independently selected from leucine, isoleucine or valine (optionally leucine) and each of A1, A2, A3 is independently selected from lysine or arginine (optionally lysine) and n and p are both 1.

Optionally both B3 and C3 are independently selected from leucine, isoleucine or valine (optionally leucine) and each of A1, A2, A3 is independently selected from lysine or arginine (optionally lysine) and n and m are both 1.

Optionally both B3 and C3 are independently selected from leucine, isoleucine or valine (optionally leucine) and each of A1, A2, A3 is independently selected from lysine or arginine (optionally lysine) and m and p are both 1.

Optionally both B3 and C3 are independently selected from leucine, isoleucine or valine (optionally leucine) and each of A1, A2, A3 is lysine or arginine (optionally lysine) and each of m, n and p are both 1.

Optionally both B3 and C3 are independently selected from leucine, isoleucine and valine (and are optionally each leucine), each of A1, A2, A3 is independently selected from lysine and arginine (and are optionally each lysine), each of m, n and p are 1 and C1 and C2 are both substituted at the α carbon with a terminal alkenyl group or terminal alkynyl group.

Optionally both B3 and C3 are independently selected from leucine, isoleucine and valine (and are optionally each leucine), each of A1, A2, A3 is independently selected from lysine or arginine (and are optionally each lysine), each of m, n and p are 1, C1 and C2 are both substituted at the α carbon with a terminal alkenyl group or terminal alkynyl group and B3 and C3 are both unsubstituted.

Optionally both B3 and C3 are independently selected from leucine, isoleucine and valine (and are optionally each leucine), each of A1, A2, A3 is independently selected from lysine and arginine (and are optionally each lysine), each of m, n and p are 1, C1 and C2 are both substituted at the α carbon with a terminal alkenyl group or terminal alkynyl group, B3 and C3 are both unsubstituted and each of B1, C1, D1, B2, C2 and D2 are independently selected from glycine, valine, alanine, isoleucine, leucine, proline and methionine (and are optionally each alanine).

Optionally both B3 and C3 are independently selected from leucine, isoleucine and valine (and are optionally each leucine), each of A1, A2, A3 is independently selected from lysine and arginine (and are optionally each lysine), each of m, n and p are 1, C1 and C2 are both substituted at the α carbon with a terminal alkenyl group or terminal alkynyl group, B3 and C3 are both unsubstituted and each of B1, C1, D1, B2, C2 and D2 are independently selected from valine, alanine, isoleucine, leucine, proline and methionine (and are optionally each alanine).

Optionally both B3 and C3 are independently selected from leucine, isoleucine and valine (and are optionally each leucine), each of A1, A2, A3 is independently selected from lysine and arginine (and are optionally each lysine), each of m, n and p are 1, C1 and C2 are both substituted at the α carbon with a terminal alkenyl group or terminal alkynyl group, B3 and C3 are both unsubstituted and each of B1, C1, D1, B2, C2 and D2 are independently selected from valine, alanine, isoleucine, leucine, and methionine (and are optionally each alanine).

Optionally both B3 and C3 are each independently selected from leucine, isoleucine and valine (and are optionally each leucine), each of A1, A2, A3 is independently selected from lysine and arginine (and are optionally each lysine), each of m, n and p are 1, C1 and C2 are both substituted at the α carbon with a terminal alkenyl group or terminal alkynyl group and B3 and C3 are both unsubstituted and each of B1, C1, D1, B2, C2 and D2 are independently selected from glycine, valine, alanine, isoleucine and leucine (and are optionally each alanine).

Optionally both B3 and C3 are each independently selected from leucine, isoleucine and valine (and are optionally each leucine), each of A1, A2, A3 is independently selected from lysine or arginine (and are optionally each lysine), each of m, n and p are 1, C1 and C2 are both substituted at the α carbon with a terminal alkenyl group or terminal alkynyl group, B3 and C3 are both unsubstituted and each of B1, C1, D1, B2, C2 and D2 are selected from valine, alanine, isoleucine and leucine (and are optionally each alanine).

Optionally both B3 and C3 are unsubstituted. Optionally C1 is substituted at the α carbon with a terminal alkenyl group or terminal alkynyl group and both B3 and C3 are unsubstituted. Optionally C2 is substituted at the α carbon with a terminal alkenyl group or terminal alkynyl group and both B3 and C3 are unsubstituted. Optionally 01 and C2 are both substituted at the α carbon with a terminal alkenyl group or terminal alkynyl group and both B3 and C3 are unsubstituted.

Optionally C1 and C2 are both substituted at the α carbon with a terminal alkenyl group or terminal alkynyl group, both B3 and C3 are unsubstituted and n is 1.

Optionally C1 and C2 are both substituted at the α carbon with a terminal alkenyl group or terminal alkynyl group, both B3 and C3 are unsubstituted and p is 1.

Optionally C1 and C2 are both substituted at the α carbon with a terminal alkenyl group or terminal alkynyl group, both B3 and C3 are unsubstituted and m is 1.

Optionally C1 and C2 are both substituted at the α carbon with a terminal alkenyl group or terminal alkynyl group, both B3 and C3 are unsubstituted, n is 1 and m is 1.

Optionally C1 and C2 are both substituted at the α carbon with a terminal alkenyl group or terminal alkynyl group, both B3 and C3 are unsubstituted, p is 1 and m is 1.

Optionally C1 and C2 are both substituted at the α carbon with a terminal alkenyl group or terminal alkynyl group, both B3 and C3 are unsubstituted, n is 1, p is 1 and m is 1.

Advantageously the third sequence is conjugated to the C-terminus of the first sequence. Optionally the third sequence is conjugated to the C-terminus of the first sequence and the N-terminus of the second sequence.

Any of the peptides of the invention may further comprise a fourth sequence A4-B4-(C4)_{q}, that is optionally conjugated to the N-terminus of the first sequence, wherein:
A4 is an amino acid that has a positively charged side chain at pH 7;
B4 is an amino acid having a hydrophobic side chain;
C4, when present, is an amino acid having a hydrophobic side chain; and
q is 0 or 1;
wherein B4 and C4 are independently (i) unsubstituted or (ii) substituted at the α carbon with a terminal alkenyl group or terminal alkynyl group.

Optionally q is 1. Optionally q is 1 and n is 1. Optionally q is 1 and p is 1. Optionally q is 1 and m is 1. Optionally q is 1, n is 1 and p is 1. Optionally q is 1, n is 1 and m is 1. Optionally q is 1, p is 1 and m is 1. Optionally q is 1, n is 1, p is 1 and m is 1.

Optionally A4 is lysine or arginine, optionally lysine.

Optionally A1 and A4 are each independently selected from lysine or arginine, optionally A1 and A4 are both lysine. Optionally A2 and A4 are each independently selected from lysine or arginine, optionally A2 and A4 are both lysine. Optionally A3 and A4 are each independently selected from lysine or arginine, optionally A3 and A4 are both lysine.

Optionally each of A1, A2 and A4 are independently selected from lysine or arginine, optionally each of A1, A2 and A4 are lysine. Optionally each of A1, A3 and A4 are independently selected from lysine or arginine, optionally each of A1, A3 and A4 are lysine. Optionally each of A2, A3 and A4 are independently selected from lysine or arginine, optionally each of A2, A3 and A4 are lysine.

Optionally each of A1, A2, A3 and A4 are independently selected from lysine or arginine, optionally each of A1, A2, A3 and A4 are lysine.

Optionally B4 is leucine, isoleucine or valine. Optionally B4 is leucine. B4 may be unsubstituted.

Optionally both B3 and B4 are each independently selected from leucine, isoleucine or valine. Optionally B3 and B4 are each leucine.

Optionally both C3 and B4 are each independently selected from leucine, isoleucine or valine. Optionally C3 and B4 are each leucine.

Optionally all of B3, C3 and B4 are each independently selected from leucine, isoleucine or valine. Optionally all of B3, C3 and B4 are leucine.

Optionally both B3 and C3 are independently selected from leucine, isoleucine or valine (optionally leucine) and A4 is lysine or arginine (optionally lysine).

Optionally both B3 and C3 are independently selected from leucine, isoleucine or valine (optionally leucine) and both A1 and A4 are independently selected from lysine or arginine (optionally lysine).

Optionally both B3 and C3 are independently selected from leucine, isoleucine or valine (optionally leucine) and both A2 and A4 are independently selected from lysine or arginine (optionally lysine).

Optionally both B3 and C3 are independently selected from leucine, isoleucine or valine (optionally leucine) and both A3 and A4 are independently selected from lysine or arginine (optionally lysine).

Optionally both B3 and C3 are independently selected from leucine, isoleucine or valine (optionally leucine) and A1, A2 and A4 are each independently selected from lysine or arginine (optionally lysine).

Optionally both B3 and C3 are independently selected from leucine, isoleucine or valine (optionally leucine) and A1, A3 and A4 are each independently selected from lysine or arginine (optionally lysine).

Optionally both B3 and C3 are independently selected from leucine, isoleucine or valine (optionally leucine) and A2, A3 and A4 are each independently selected from lysine or arginine (optionally lysine).

Optionally both B3 and C3 are independently selected from leucine, isoleucine or valine (optionally leucine) and A1, A2, A3 and A4 are each independently selected from lysine or arginine (optionally lysine).

Optionally both B3 and C3 are independently selected from leucine, isoleucine or valine (optionally leucine) and each of A1, A2, A3 is independently selected from lysine or arginine (optionally lysine) and each of m, n and p, q are both 1.

Optionally B4, B3 and C3 are each independently selected from leucine, isoleucine and valine (and are optionally each leucine), each of A1, A2, A3 and A4 is independently selected from lysine and arginine (and are optionally each lysine), each of m, n, p and q are 1, C1 and C2 are both substituted at the α carbon with a terminal alkenyl group or terminal alkynyl group, B3, C3, B4 and C4 are all unsubstituted and each of B1, C1, D1, B2, C2, D2 and C4 are each independently selected from valine, alanine, isoleucine and leucine (and are optionally each alanine).

Optionally q is 1 and C4 is unsubstituted.

Optionally both B4 and C4 are unsubstituted. Optionally C1 is substituted at the α carbon with a terminal alkenyl group or terminal alkynyl group and both B4 and C4 are unsubstituted. Optionally C2 is substituted at the α carbon with a terminal alkenyl group or terminal alkynyl group and both B4 and C4 are unsubstituted. Optionally C1 and C2 are both substituted at the α carbon with a terminal alkenyl group or terminal alkynyl group and both B4 and C4 are unsubstituted.

Optionally B3, C3, B4 and C4 are all unsubstituted. Optionally C1 is substituted at the α carbon with a terminal alkenyl group or terminal alkynyl group and B3, C3, B4 and C4 are all unsubstituted. Optionally C2 is substituted at the α carbon with a terminal alkenyl group or terminal alkynyl group and B3, C3, B4 and C4 are all unsubstituted. Optionally C1 and C2 are both substituted at the α carbon with a terminal alkenyl group or terminal alkynyl group and B3, C3, B4 and C4 are all unsubstituted. Optionally C1 and C2 are both substituted at the α carbon with a terminal alkenyl group or terminal alkynyl group, both B3 and C3 are unsubstituted and r is 0.

Optionally C1 and C2 are both substituted at the α carbon with a terminal alkenyl group or terminal alkynyl group, both B3 and C3 are unsubstituted and q is 1.

Optionally C1 and C2 are both substituted at the α carbon with a terminal alkenyl group or terminal alkynyl group, both B3 and C3 are unsubstituted, q is 1 and n is 1.

Optionally C1 and C2 are both substituted at the α carbon with a terminal alkenyl group or terminal alkynyl group, both B3 and C3 are unsubstituted, q is 1 and p is 1.

Optionally C1 and C2 are both substituted at the α carbon with a terminal alkenyl group or terminal alkynyl group, both B3 and C3 are unsubstituted, q is 1 and m is 1.

Optionally C1 and C2 are both substituted at the α carbon with a terminal alkenyl group or terminal alkynyl group, both B3 and C3 are unsubstituted, n is 1 and p is 1.

Optionally C1 and C2 are both substituted at the α carbon with a terminal alkenyl group or terminal alkynyl group, both B3 and C3 are unsubstituted, q is 1, n is 1 and p is 1.

Optionally C1 and C2 are both substituted at the α carbon with a terminal alkenyl group or terminal alkynyl group, both B3 and C3 are unsubstituted, q is 1, n is 1 and m is 1.

Optionally C1 and C2 are both substituted at the α carbon with a terminal alkenyl group or terminal alkynyl group, both B3 and C3 are unsubstituted, q is 1, p is 1 and m is 1.

Optionally C1 and C2 are both substituted at the α carbon with a terminal alkenyl group or terminal alkynyl group, both B3 and C3 are unsubstituted, q is 1, n is 1, p is 1, and m is 1.

Optionally C1 and C2 are both substituted at the α carbon with a terminal alkenyl group or terminal alkynyl group, both B4 and C4 are unsubstituted and q is 1.

Optionally C1 and C2 are both substituted at the α carbon with a terminal alkenyl group or terminal alkynyl group, both B4 and C4 are unsubstituted and n is 1.

Optionally C1 and C2 are both substituted at the α carbon with a terminal alkenyl group or terminal alkynyl group, both B4 and C4 are unsubstituted and p is 1.

Optionally C1 and C2 are both substituted at the α carbon with a terminal alkenyl group or terminal alkynyl group, both B4 and C4 are unsubstituted and m is 1.

Optionally C1 and C2 are both substituted at the α carbon with a terminal alkenyl group or terminal alkynyl group, both B4 and C4 are unsubstituted, q is 1 and n is 1.

Optionally C1 and C2 are both substituted at the α carbon with a terminal alkenyl group or terminal alkynyl group, both B4 and C4 are unsubstituted, q is 1 and p is 1.

Optionally C1 and C2 are both substituted at the α carbon with a terminal alkenyl group or terminal alkynyl group, both B4 and C4 are unsubstituted, q is 1 and m is 1.

Optionally C1 and C2 are both substituted at the α carbon with a terminal alkenyl group or terminal alkynyl group, both B4 and C4 are unsubstituted, n is 1 and p is 1.

Optionally C1 and C2 are both substituted at the α carbon with a terminal alkenyl group or terminal alkynyl group, both B4 and C4 are unsubstituted, n is 1 and m is 1.

Optionally C1 and C2 are both substituted at the α carbon with a terminal alkenyl group or terminal alkynyl group, both B4 and C4 are unsubstituted, p is 1 and m is 1.

Optionally C1 and C2 are both substituted at the α carbon with a terminal alkenyl group or terminal alkynyl group, both B4 and C4 are unsubstituted, q is 1, n is 1 and p is 1.

Optionally C1 and C2 are both substituted at the α carbon with a terminal alkenyl group or terminal alkynyl group, both B4 and C4 are unsubstituted, q is 1, n is 1 and m is 1.

Optionally C1 and C2 are both substituted at the α carbon with a terminal alkenyl group or terminal alkynyl group, both B4 and C4 are unsubstituted, q is 1, p is 1 and m is 1.

Optionally C1 and C2 are both substituted at the α carbon with a terminal alkenyl group or terminal alkynyl group, both B4 and C4 are unsubstituted, n is 1, p is 1 and m is 1.

Optionally C1 and C2 are both substituted at the α carbon with a terminal alkenyl group or terminal alkynyl group, both B4 and C4 are unsubstituted, q is 1, n is 1, p is 1, and m is 1.

Optionally C1 and C2 are both substituted at the α carbon with a terminal alkenyl group or terminal alkynyl group, all of B3, C3, B4 and C4 are unsubstituted and q is 1.

Optionally C1 and C2 are both substituted at the α carbon with a terminal alkenyl group or terminal alkynyl group, all of B3, C3, B4 and C4 are unsubstituted and n is 1.

Optionally C1 and C2 are both substituted at the α carbon with a terminal alkenyl group or terminal alkynyl group, all of B3, C3, B4 and C4 are unsubstituted and p is 1.

Optionally C1 and C2 are both substituted at the α carbon with a terminal alkenyl group or terminal alkynyl group, all of B3, C3, B4 and C4 are unsubstituted and m is 1.

Optionally C1 and C2 are both substituted at the α carbon with a terminal alkenyl group or terminal alkynyl group, all of B3, C3, B4 and C4 are unsubstituted, q is 1 and n is 1.

Optionally C1 and C2 are both substituted at the α carbon with a terminal alkenyl group or terminal alkynyl group, all of B3, C3, B4 and C4 are unsubstituted, q is 1 and p is 1.

Optionally C1 and C2 are both substituted at the α carbon with a terminal alkenyl group or terminal alkynyl group, all of B3, C3, B4 and C4 are unsubstituted, q is 1 and m is 1.

Optionally C1 and C2 are both substituted at the α carbon with a terminal alkenyl group or terminal alkynyl group, all of B3, C3, B4 and C4 are unsubstituted, n is 1 and p is 1.

Optionally C1 and C2 are both substituted at the α carbon with a terminal alkenyl group or terminal alkynyl group, all of B3, C3, B4 and C4 are unsubstituted, n is 1 and m is 1.

Optionally C1 and C2 are both substituted at the α carbon with a terminal alkenyl group or terminal alkynyl group, all of B3, C3, B4 and C4 are unsubstituted, p is 1 and m is 1.

Optionally C1 and C2 are both substituted at the α carbon with a terminal alkenyl group or terminal alkynyl group, all of B3, C3, B4 and C4 are unsubstituted, q is 1, n is 1 and p is 1.

Optionally C1 and C2 are both substituted at the α carbon with a terminal alkenyl group or terminal alkynyl group, all of B3, C3, B4 and C4 are unsubstituted, q is 1, n is 1 and m is 1.

Optionally C1 and C2 are both substituted at the α carbon with a terminal alkenyl group or terminal alkynyl group, all of B3, C3, B4 and C4 are unsubstituted, q is 1, p is 1 and m is 1.

Optionally C1 and C2 are both substituted at the α carbon with a terminal alkenyl group or terminal alkynyl group, all of B3, C3, B4 and C4 are unsubstituted, n is 1, p is 1 and m is 1.

Optionally C1 and C2 are both substituted at the α carbon with a terminal alkenyl group or terminal alkynyl group, all of B3, C3, B4 and C4 are unsubstituted, r is 0, q is 1, p is 1, and m is 1.

Optionally C1 and C2 are both substituted at the α carbon with a terminal alkenyl group or terminal alkynyl group, all of B3, C3, B4 and C4 are unsubstituted, r is 0, n is 1, p is 1, and m is 1.

Optionally C1 and C2 are both substituted at the α carbon with a terminal alkenyl group or terminal alkynyl group, all of B3, C3, B4 and C4 are unsubstituted, r is 0, q is 1, n is 1, p is 1 and m is 1.

Optionally C1 and C2 are both substituted at the α carbon with a terminal alkenyl group or terminal alkynyl group, all of B3, C3, B4 and C4 are unsubstituted, q is 1, n is 1, p is 1, and m is 1.

Advantageously the fourth sequence is conjugated to the N-terminus of the first sequence. Alternatively, the fourth sequence may be conjugated to the C-terminus of the first sequence.

As used herein, the term “conjugated” can include both directly conjugated and indirectly conjugated. Advantageously it means directly conjugated; suitably, two sequences as described herein may be attached to each other simply via a peptide bond. For example, the third sequence can be attached directly to the C-terminus of the first sequence by a peptide bond and/or the fourth sequence can be attached directly to the N-terminus of the first sequence by a peptide bond. The term “indirectly”, when used in conjunction with “conjugated” or such like, can refer to a connection which is achieved using a linker. For example, a linker can be used to indirectly attach one sequence as described herein to another, according to some embodiments. The linker may comprise one or more amino acids and/or sequences as disclosed herein. Merely by way of illustration, therefore, if the third and fourth sequences are both conjugated to the C-terminus of the first sequence in an embodiment, the third sequence may be attached to the C-terminus of the first sequence directly (via a peptide bond) and the fourth sequence may be attached to the C-terminus of the first sequence indirectly (via a linker that comprises the third sequence).

Those peptides of the invention that comprise a fourth sequence may further comprise a fifth sequence B5-C5-(D5)ᵣ, that is optionally conjugated to the N-terminus of the fourth sequence, wherein:
B5 is an amino acid having a hydrophobic side chain;
C5 is an amino acid having a hydrophobic side chain;
D5, when present, is an amino acid having a hydrophobic side chain; and
r is 0 or 1;
wherein B5 and C5 are independently (i) unsubstituted or (ii) substituted at the α carbon with a terminal alkenyl group or terminal alkynyl group.

Advantageously the fifth sequence is conjugated to the N-terminus of the fourth sequence. Alternatively, the fifth sequence may be conjugated to the C-terminus of the fourth sequence.

Optionally r is 0. Optionally r is 0, q is 1, n is 1 and p is 1. Optionally r is 0, q is 1, n is 1 and m is 1. Optionally r is 0, q is 1, p is 1 and m is 1. Optionally r is 0, n is 1, p is 1 and m is 1. Optionally r is 0, q is 1, n is 1, p is 1 and m is 1.

Optionally C1 and C2 are both substituted at the α carbon with a terminal alkenyl group or terminal alkynyl group, both B3 and C3 are unsubstituted, r is 0 and q is 1.

Optionally C1 and C2 are both substituted at the α carbon with a terminal alkenyl group or terminal alkynyl group, both B3 and C3 are unsubstituted, r is 0 and n is 1.

Optionally C1 and C2 are both substituted at the α carbon with a terminal alkenyl group or terminal alkynyl group, both B3 and C3 are unsubstituted, r is 0 and p is 1.

Optionally C1 and C2 are both substituted at the α carbon with a terminal alkenyl group or terminal alkynyl group, both B3 and C3 are unsubstituted, r is 0 and m is 1.

Optionally C1 and C2 are both substituted at the α carbon with a terminal alkenyl group or terminal alkynyl group, both B3 and C3 are unsubstituted, r is 0, q is 1 and n is 1.

Optionally C1 and C2 are both substituted at the α carbon with a terminal alkenyl group or terminal alkynyl group, both B3 and C3 are unsubstituted, r is 0, q is 1 and p is 1.

Optionally C1 and C2 are both substituted at the α carbon with a terminal alkenyl group or terminal alkynyl group, both B3 and C3 are unsubstituted, r is 0, q is 1 and m is 1.

Optionally C1 and C2 are both substituted at the α carbon with a terminal alkenyl group or terminal alkynyl group, both B3 and C3 are unsubstituted, r is 0, n is 1 and p is 1.

Optionally C1 and C2 are both substituted at the α carbon with a terminal alkenyl group or terminal alkynyl group, both B3 and C3 are unsubstituted, r is 0, n is 1 and m is 1.

Optionally C1 and C2 are both substituted at the α carbon with a terminal alkenyl group or terminal alkynyl group, both B3 and C3 are unsubstituted, r is 0, p is 1 and m is 1.

Optionally C1 and C2 are both substituted at the α carbon with a terminal alkenyl group or terminal alkynyl group, both B3 and C3 are unsubstituted, r is 0, q is 1, n is 1, and p is 1.

Optionally C1 and C2 are both substituted at the α carbon with a terminal alkenyl group or terminal alkynyl group, both B3 and C3 are unsubstituted, r is 0, q is 1, n is 1, and m is 1.

Optionally C1 and C2 are both substituted at the α carbon with a terminal alkenyl group or terminal alkynyl group, both B3 and C3 are unsubstituted, r is 0, q is 1, n is 1, and m is 1.

Optionally C1 and C2 are both substituted at the α carbon with a terminal alkenyl group or terminal alkynyl group, both B3 and C3 are unsubstituted, r is 0, q is 1, p is 1, and m is 1.

Optionally C1 and C2 are both substituted at the α carbon with a terminal alkenyl group or terminal alkynyl group, both B3 and C3 are unsubstituted, r is 0, n is 1, p is 1, and m is 1.

Optionally C1 and C2 are both substituted at the α carbon with a terminal alkenyl group or terminal alkynyl group, both B3 and C3 are unsubstituted, r is 0, q is 1, n is 1, p is 1 and m is 1.

Optionally C1 and C2 are both substituted at the α carbon with a terminal alkenyl group or terminal alkynyl group, both B4 and C4 are unsubstituted and r is 0.

Optionally C1 and C2 are both substituted at the α carbon with a terminal alkenyl group or terminal alkynyl group, both B4 and C4 are unsubstituted, r is 0 and q is 1.

Optionally C1 and C2 are both substituted at the α carbon with a terminal alkenyl group or terminal alkynyl group, both B4 and C4 are unsubstituted, r is 0 and n is 1.

Optionally C1 and C2 are both substituted at the α carbon with a terminal alkenyl group or terminal alkynyl group, both B4 and C4 are unsubstituted, r is 0 and p is 1.

Optionally C1 and C2 are both substituted at the α carbon with a terminal alkenyl group or terminal alkynyl group, both B4 and C4 are unsubstituted, r is 0 and m is 1.

Optionally C1 and C2 are both substituted at the α carbon with a terminal alkenyl group or terminal alkynyl group, both B4 and C4 are unsubstituted, r is 0, q is 1 and n is 1.

Optionally C1 and C2 are both substituted at the α carbon with a terminal alkenyl group or terminal alkynyl group, both B4 and C4 are unsubstituted, r is 0, q is 1 and p is 1.

Optionally C1 and C2 are both substituted at the α carbon with a terminal alkenyl group or terminal alkynyl group, both B4 and C4 are unsubstituted, r is 0, q is 1 and m is 1.

Optionally C1 and C2 are both substituted at the α carbon with a terminal alkenyl group or terminal alkynyl group, both B4 and C4 are unsubstituted, r is 0, n is 1 and p is 1.

Optionally C1 and C2 are both substituted at the α carbon with a terminal alkenyl group or terminal alkynyl group, both B4 and C4 are unsubstituted, r is 0, n is 1 and m is 1.

Optionally C1 and C2 are both substituted at the α carbon with a terminal alkenyl group or terminal alkynyl group, both B4 and C4 are unsubstituted, r is 0, p is 1 and m is 1.

Optionally C1 and C2 are both substituted at the α carbon with a terminal alkenyl group or terminal alkynyl group, both B4 and C4 are unsubstituted, r is 0, q is 1, n is 1, and p is 1.

Optionally C1 and C2 are both substituted at the α carbon with a terminal alkenyl group or terminal alkynyl group, both B4 and C4 are unsubstituted, r is 0, q is 1, n is 1, and m is 1.

Optionally C1 and C2 are both substituted at the α carbon with a terminal alkenyl group or terminal alkynyl group, both B4 and C4 are unsubstituted, r is 0, q is 1, n is 1, and m is 1.

Optionally C1 and C2 are both substituted at the α carbon with a terminal alkenyl group or terminal alkynyl group, both B4 and C4 are unsubstituted, r is 0, q is 1, p is 1, and m is 1.

Optionally C1 and C2 are both substituted at the α carbon with a terminal alkenyl group or terminal alkynyl group, both B4 and C4 are unsubstituted, r is 0, n is 1, p is 1, and m is 1.

Optionally C1 and C2 are both substituted at the α carbon with a terminal alkenyl group or terminal alkynyl group, both B4 and C4 are unsubstituted, r is 0, q is 1, n is 1, p is 1 and m is 1.

Optionally C1 and C2 are both substituted at the α carbon with a terminal alkenyl group or terminal alkynyl group, all of B3, C3, B4 and C4 are unsubstituted and r is 0.

Optionally C1 and C2 are both substituted at the α carbon with a terminal alkenyl group or terminal alkynyl group, all of B3, C3, B4 and C4 are unsubstituted, r is 0 and q is 1.

Optionally C1 and C2 are both substituted at the α carbon with a terminal alkenyl group or terminal alkynyl group, all of B3, C3, B4 and C4 are unsubstituted, r is 0 and n is 1.

Optionally C1 and C2 are both substituted at the α carbon with a terminal alkenyl group or terminal alkynyl group, all of B3, C3, B4 and C4 are unsubstituted, r is 0 and p is 1.

Optionally C1 and C2 are both substituted at the α carbon with a terminal alkenyl group or terminal alkynyl group, all of B3, C3, B4 and C4 are unsubstituted, r is 0 and m is 1.

Optionally C1 and C2 are both substituted at the α carbon with a terminal alkenyl group or terminal alkynyl group, all of B3, C3, B4 and C4 are unsubstituted, r is 0, q is 1 and n is 1.

Optionally C1 and C2 are both substituted at the α carbon with a terminal alkenyl group or terminal alkynyl group, all of B3, C3, B4 and C4 are unsubstituted, r is 0, q is 1 and p is 1.

Optionally C1 and C2 are both substituted at the α carbon with a terminal alkenyl group or terminal alkynyl group, all of B3, C3, B4 and C4 are unsubstituted, r is 0, q is 1 and m is 1.

Optionally C1 and C2 are both substituted at the α carbon with a terminal alkenyl group or terminal alkynyl group, all of B3, C3, B4 and C4 are unsubstituted, r is 0, n is 1 and p is 1.

Optionally C1 and C2 are both substituted at the α carbon with a terminal alkenyl group or terminal alkynyl group, all of B3, C3, B4 and C4 are unsubstituted, r is 0, n is 1 and m is 1.

Optionally C1 and C2 are both substituted at the α carbon with a terminal alkenyl group or terminal alkynyl group, all of B3, C3, B4 and C4 are unsubstituted, r is 0, p is 1 and m is 1.

Optionally C1 and C2 are both substituted at the α carbon with a terminal alkenyl group or terminal alkynyl group, all of B3, C3, B4 and C4 are unsubstituted, r is 0, q is 1, n is 1, and p is 1.

Optionally C1 and C2 are both substituted at the α carbon with a terminal alkenyl group or terminal alkynyl group, all of B3, C3, B4 and C4 are unsubstituted, r is 0, q is 1, n is 1, and m is 1.

Optionally C1 and C2 are both substituted at the α carbon with a terminal alkenyl group or terminal alkynyl group, all of B3, C3, B4 and C4 are unsubstituted, r is 0, q is 1, n is 1, and m is 1.

Optionally B5 is leucine, isoleucine or valine. Optionally B5 is leucine. B5 may be unsubstituted.

Optionally both B3 and B5 are each independently selected from leucine, isoleucine or valine. Optionally B3 and B5 are each leucine.

Optionally both C3 and B5 are each independently selected from leucine, isoleucine or valine. Optionally C3 and B5 are each leucine.

Optionally both B4 and B5 are each independently selected from leucine, isoleucine or valine. Optionally B4 and B5 are each leucine.

Optionally all of B3, C3 and B5 are each independently selected from leucine, isoleucine or valine. Optionally all of B3, C3 and B5 are leucine.

Optionally all of B3, B4 and B5 are each independently selected from leucine, isoleucine or valine. Optionally all of B3, B4 and B5 are leucine.

Optionally all of C3, B4 and B5 are each independently selected from leucine, isoleucine or valine. Optionally all of C3, B4 and B5 are leucine.

Optionally all of B3, C3, B4, and B5 are each independently selected from leucine, isoleucine or valine. Optionally all of B3, C3, B4, and B5 are leucine.

Optionally C5 is unsubstituted. Alternatively, C5 may be substituted at the α carbon with a terminal alkenyl group or terminal alkynyl group.

Any of the peptides of the invention may further comprise a sixth sequence A6-B6-C6-(D6)ₛ, that is optionally conjugated to the C-terminus of the second sequence, wherein:
A6 is an amino acid that has a positively charged side chain at pH 7;
B6 is an amino acid having a hydrophobic side chain;
C6 is an amino acid having a hydrophobic side chain;
D6, when present, is an amino acid having a hydrophobic side chain; and
s is 0 or 1;
wherein B6 and C6 are independently (i) unsubstituted or (ii) substituted at the α carbon with a terminal alkenyl group or terminal alkynyl group.

Advantageously the sixth sequence is conjugated to the C-terminus of the second sequence. Alternatively, the sixth sequence may be conjugated to the N-terminus of the second sequence.

Optionally s is 0. Optionally s is 0, q is 1, n is 1 and p is 1. Optionally s is 0, q is 1, n is 1 and m is 1. Optionally s is 0, q is 1, p is 1 and m is 1. Optionally s is 0, n is 1, p is 1 and m is 1. Optionally s is 0, q is 1, n is 1, p is 1 and m is 1. Optionally r is 0, s is 0, q is 1, n is 1 and p is 1. Optionally r is 0, s is 0, q is 1, n is 1 and m is 1. Optionally r is 0, s is 0, q is 1, p is 1 and m is 1. Optionally r is 0, s is 0, n is 1, p is 1 and m is 1. Optionally r is 0, s is 0, q is 1, n is 1, p is 1 and m is 1.

Optionally A6 is lysine or arginine, optionally lysine.

Optionally A1 and A6 are each independently selected from lysine or arginine, optionally A1 and A6 are both lysine. Optionally A2 and A6 are each independently selected from lysine or arginine, optionally A2 and A6 are both lysine. Optionally A3 and A6 are each independently selected from lysine or arginine, optionally A3 and A6 are both lysine. Optionally A4 and A6 are each independently selected from lysine or arginine, optionally A4 and A6 are both lysine.

Optionally each of A1, A2 and A6 are independently selected from lysine or arginine, optionally each of A1, A2 and A6 are lysine. Optionally each of A1, A3 and A6 are independently selected from lysine or arginine, optionally each of A1, A3 and A6 are lysine. Optionally each of A1, A4 and A6 are independently selected from lysine or arginine, optionally each of A1, A4 and A6 are lysine. Optionally each of A2, A3 and A6 are independently selected from lysine or arginine, optionally each of A2, A3 and A6 are lysine. Optionally each of A2, A4 and A6 are independently selected from lysine or arginine, optionally each of A2, A4 and A6 are lysine. Optionally each of A3, A4 and A6 are independently selected from lysine or arginine, optionally each of A3, A4 and A6 are lysine.

Optionally each of A1, A2, A3 and A6 are independently selected from lysine or arginine, optionally each of A1, A2, A3 and A6 are lysine. Optionally each of A1, A2, A4 and A6 are independently selected from lysine or arginine, optionally each of A1, A2, A4 and A6 are lysine. Optionally each of A1, A3, A4 and A6 are independently selected from lysine or arginine, optionally each of A1, A3, A4 and A6 are lysine. Optionally each of A2, A3, A4 and A6 are independently selected from lysine or arginine, optionally each of A2, A3, A4 and A6 are lysine.

Optionally each of A1, A2, A3, A4 and A6 are independently selected from lysine or arginine, optionally each of A1, A2, A3, A4 and A6 are lysine.

Optionally C1 and C2 are both substituted at the α carbon with a terminal alkenyl group or terminal alkynyl group, both B3 and C3 are unsubstituted and s is 0.

Optionally C1 and C2 are both substituted at the α carbon with a terminal alkenyl group or terminal alkynyl group, both B3 and C3 are unsubstituted, s is 0 and q is 1.

Optionally C1 and C2 are both substituted at the α carbon with a terminal alkenyl group or terminal alkynyl group, both B3 and C3 are unsubstituted, s is 0 and n is 1.

Optionally C1 and C2 are both substituted at the α carbon with a terminal alkenyl group or terminal alkynyl group, both B3 and C3 are unsubstituted, s is 0 and p is 1.

Optionally C1 and C2 are both substituted at the α carbon with a terminal alkenyl group or terminal alkynyl group, both B3 and C3 are unsubstituted, s is 0 and m is 1.

Optionally C1 and C2 are both substituted at the α carbon with a terminal alkenyl group or terminal alkynyl group, both B3 and C3 are unsubstituted, r is 0 and s is 0.

Optionally C1 and C2 are both substituted at the α carbon with a terminal alkenyl group or terminal alkynyl group, both B3 and C3 are unsubstituted, r is 0, q is 1 and s is 0.

Optionally C1 and C2 are both substituted at the α carbon with a terminal alkenyl group or terminal alkynyl group, both B3 and C3 are unsubstituted, r is 0, n is 1 and s is 0.

Optionally C1 and C2 are both substituted at the α carbon with a terminal alkenyl group or terminal alkynyl group, both B3 and C3 are unsubstituted, r is 0, p is 1 and s is 0.

Optionally C1 and C2 are both substituted at the α carbon with a terminal alkenyl group or terminal alkynyl group, both B3 and C3 are unsubstituted, r is 0, m is 1 and s is 0.

Optionally C1 and C2 are both substituted at the α carbon with a terminal alkenyl group or terminal alkynyl group, both B3 and C3 are unsubstituted, r is 0, q is 1, n is 1, and s is 0.

Optionally C1 and C2 are both substituted at the α carbon with a terminal alkenyl group or terminal alkynyl group, both B3 and C3 are unsubstituted, r is 0, q is 1, p is 1, and s is 0.

Optionally C1 and C2 are both substituted at the α carbon with a terminal alkenyl group or terminal alkynyl group, both B3 and C3 are unsubstituted, r is 0, q is 1, m is 1, and s is 0.

Optionally C1 and C2 are both substituted at the α carbon with a terminal alkenyl group or terminal alkynyl group, both B3 and C3 are unsubstituted, r is 0, n is 1, p is 1, and s is 0.

Optionally C1 and C2 are both substituted at the α carbon with a terminal alkenyl group or terminal alkynyl group, both B3 and C3 are unsubstituted, r is 0, n is 1, m is 1, and s is 0.

Optionally C1 and C2 are both substituted at the α carbon with a terminal alkenyl group or terminal alkynyl group, both B3 and C3 are unsubstituted, r is 0, p is 1, m is 1, and s is 0.

Optionally C1 and C2 are both substituted at the α carbon with a terminal alkenyl group or terminal alkynyl group, both B3 and C3 are unsubstituted, q is 1, n is 1, p is 1, and s is 0.

Optionally C1 and C2 are both substituted at the α carbon with a terminal alkenyl group or terminal alkynyl group, both B3 and C3 are unsubstituted, q is 1, p is 1, m is 1, and s is 0.

Optionally C1 and C2 are both substituted at the α carbon with a terminal alkenyl group or terminal alkynyl group, both B3 and C3 are unsubstituted, n is 1, p is 1, m is 1, and s is 0.

Optionally C1 and C2 are both substituted at the α carbon with a terminal alkenyl group or terminal alkynyl group, both B3 and C3 are unsubstituted, r is 0, q is 1, n is 1, p is 1 and s is 0.

Optionally C1 and C2 are both substituted at the α carbon with a terminal alkenyl group or terminal alkynyl group, both B3 and C3 are unsubstituted, r is 0, q is 1, n is 1, m is 1 and s is 0.

Optionally C1 and C2 are both substituted at the α carbon with a terminal alkenyl group or terminal alkynyl group, both B3 and C3 are unsubstituted, r is 0, q is 1, p is 1, m is 1 and s is 0.

Optionally C1 and C2 are both substituted at the α carbon with a terminal alkenyl group or terminal alkynyl group, both B3 and C3 are unsubstituted, r is 0, n is 1, p is 1, m is 1 and s is 0.

Optionally C1 and C2 are both substituted at the α carbon with a terminal alkenyl group or terminal alkynyl group, both B3 and C3 are unsubstituted, q is 1, n is 1, p is 1, m is 1 and s is 0.

Optionally C1 and C2 are both substituted at the α carbon with a terminal alkenyl group or terminal alkynyl group, both B3 and C3 are unsubstituted, r is 0, s is 0, q is 1, n is 1, p is 1 and m is 1.

Optionally C1 and C2 are both substituted at the α carbon with a terminal alkenyl group or terminal alkynyl group, both B4 and C4 are unsubstituted and s is 0.

Optionally C1 and C2 are both substituted at the α carbon with a terminal alkenyl group or terminal alkynyl group, both B4 and C4 are unsubstituted, s is 0 and q is 1.

Optionally C1 and C2 are both substituted at the α carbon with a terminal alkenyl group or terminal alkynyl group, both B4 and C4 are unsubstituted, s is 0 and n is 1.

Optionally C1 and C2 are both substituted at the α carbon with a terminal alkenyl group or terminal alkynyl group, both B4 and C4 are unsubstituted, s is 0 and p is 1.

Optionally C1 and C2 are both substituted at the α carbon with a terminal alkenyl group or terminal alkynyl group, both B4 and C4 are unsubstituted, s is 0 and m is 1.

Optionally C1 and C2 are both substituted at the α carbon with a terminal alkenyl group or terminal alkynyl group, both B4 and C4 are unsubstituted, r is 0 and s is 0.

Optionally C1 and C2 are both substituted at the α carbon with a terminal alkenyl group or terminal alkynyl group, both B4 and C4 are unsubstituted, r is 0, q is 1 and s is 0.

Optionally C1 and C2 are both substituted at the α carbon with a terminal alkenyl group or terminal alkynyl group, both B4 and C4 are unsubstituted, r is 0, n is 1 and s is 0.

Optionally C1 and C2 are both substituted at the α carbon with a terminal alkenyl group or terminal alkynyl group, both B4 and C4 are unsubstituted, r is 0, p is 1 and s is 0.

Optionally C1 and C2 are both substituted at the α carbon with a terminal alkenyl group or terminal alkynyl group, both B4 and C4 are unsubstituted, r is 0, m is 1 and s is 0.

Optionally C1 and C2 are both substituted at the α carbon with a terminal alkenyl group or terminal alkynyl group, both B4 and C4 are unsubstituted, r is 0, q is 1, n is 1, and s is 0.

Optionally C1 and C2 are both substituted at the α carbon with a terminal alkenyl group or terminal alkynyl group, both B4 and C4 are unsubstituted, r is 0, q is 1, p is 1, and s is 0.

Optionally C1 and C2 are both substituted at the α carbon with a terminal alkenyl group or terminal alkynyl group, both B4 and C4 are unsubstituted, r is 0, q is 1, m is 1, and s is 0.

Optionally C1 and C2 are both substituted at the α carbon with a terminal alkenyl group or terminal alkynyl group, both B4 and C4 are unsubstituted, r is 0, n is 1, p is 1, and s is 0.

Optionally C1 and C2 are both substituted at the α carbon with a terminal alkenyl group or terminal alkynyl group, both B4 and C4 are unsubstituted, r is 0, n is 1, m is 1, and s is 0.

Optionally C1 and C2 are both substituted at the α carbon with a terminal alkenyl group or terminal alkynyl group, both B4 and C4 are unsubstituted, r is 0, p is 1, m is 1, and s is 0.

Optionally C1 and C2 are both substituted at the α carbon with a terminal alkenyl group or terminal alkynyl group, both B4 and C4 are unsubstituted, q is 1, n is 1, p is 1, and s is 0.

Optionally C1 and C2 are both substituted at the α carbon with a terminal alkenyl group or terminal alkynyl group, both B4 and C4 are unsubstituted, q is 1, p is 1, m is 1, and s is 0.

Optionally C1 and C2 are both substituted at the α carbon with a terminal alkenyl group or terminal alkynyl group, both B4 and C4 are unsubstituted, n is 1, p is 1, m is 1, and s is 0.

Optionally C1 and C2 are both substituted at the α carbon with a terminal alkenyl group or terminal alkynyl group, both B4 and C4 are unsubstituted, r is 0, q is 1, n is 1, p is 1 and s is 0.

Optionally C1 and C2 are both substituted at the α carbon with a terminal alkenyl group or terminal alkynyl group, both B4 and C4 are unsubstituted, r is 0, q is 1, n is 1, m is 1 and s is 0.

Optionally C1 and C2 are both substituted at the α carbon with a terminal alkenyl group or terminal alkynyl group, both B4 and C4 are unsubstituted, r is 0, q is 1, p is 1, m is 1 and s is 0.

Optionally C1 and C2 are both substituted at the α carbon with a terminal alkenyl group or terminal alkynyl group, both B4 and C4 are unsubstituted, r is 0, n is 1, p is 1, m is 1 and s is 0.

Optionally C1 and C2 are both substituted at the α carbon with a terminal alkenyl group or terminal alkynyl group, both B4 and C4 are unsubstituted, q is 1, n is 1, p is 1, m is 1 and s is 0.

Optionally C1 and C2 are both substituted at the α carbon with a terminal alkenyl group or terminal alkynyl group, both B4 and C4 are unsubstituted, r is 0, s is 0, q is 1, n is 1, p is 1 and m is 1.

Optionally C1 and C2 are both substituted at the α carbon with a terminal alkenyl group or terminal alkynyl group, all of B3, C3, B4 and C4 are unsubstituted and s is 0.

Optionally C1 and C2 are both substituted at the α carbon with a terminal alkenyl group or terminal alkynyl group, all of B3, C3, B4 and C4 are unsubstituted, s is 0 and q is 1.

Optionally C1 and C2 are both substituted at the α carbon with a terminal alkenyl group or terminal alkynyl group, all of B3, C3, B4 and C4 are unsubstituted, s is 0 and n is 1.

Optionally C1 and C2 are both substituted at the α carbon with a terminal alkenyl group or terminal alkynyl group, all of B3, C3, B4 and C4 are unsubstituted, s is 0 and p is 1.

Optionally C1 and C2 are both substituted at the α carbon with a terminal alkenyl group or terminal alkynyl group, all of B3, C3, B4 and C4 are unsubstituted, s is 0 and m is 1.

Optionally C1 and C2 are both substituted at the α carbon with a terminal alkenyl group or terminal alkynyl group, all of B3, C3, B4 and C4 are unsubstituted, r is 0 and s is 0.

Optionally C1 and C2 are both substituted at the α carbon with a terminal alkenyl group or terminal alkynyl group, all of B3, C3, B4 and C4 are unsubstituted, r is 0, q is 1 and s is 0.

Optionally C1 and C2 are both substituted at the α carbon with a terminal alkenyl group or terminal alkynyl group, all of B3, C3, B4 and C4 are unsubstituted, r is 0, n is 1 and s is 0.

Optionally C1 and C2 are both substituted at the α carbon with a terminal alkenyl group or terminal alkynyl group, all of B3, C3, B4 and C4 are unsubstituted, r is 0, p is 1 and s is 0.

Optionally C1 and C2 are both substituted at the α carbon with a terminal alkenyl group or terminal alkynyl group, all of B3, C3, B4 and C4 are unsubstituted, r is 0, m is 1 and s is 0.

Optionally C1 and C2 are both substituted at the α carbon with a terminal alkenyl group or terminal alkynyl group, all of B3, C3, B4 and C4 are unsubstituted, r is 0, q is 1, n is 1, and s is 0.

Optionally C1 and C2 are both substituted at the α carbon with a terminal alkenyl group or terminal alkynyl group, all of B3, C3, B4 and C4 are unsubstituted, r is 0, q is 1, p is 1, and s is 0.

Optionally C1 and C2 are both substituted at the α carbon with a terminal alkenyl group or terminal alkynyl group, all of B3, C3, B4 and C4 are unsubstituted, r is 0, q is 1, m is 1, and s is 0.

Optionally C1 and C2 are both substituted at the α carbon with a terminal alkenyl group or terminal alkynyl group, all of B3, C3, B4 and C4 are unsubstituted, r is 0, n is 1, p is 1, and s is 0.

Optionally C1 and C2 are both substituted at the α carbon with a terminal alkenyl group or terminal alkynyl group, all of B3, C3, B4 and C4 are unsubstituted, r is 0, n is 1, m is 1, and s is 0.

Optionally C1 and C2 are both substituted at the α carbon with a terminal alkenyl group or terminal alkynyl group, all of B3, C3, B4 and C4 are unsubstituted, r is 0, p is 1, m is 1, and s is 0.

Optionally C1 and C2 are both substituted at the α carbon with a terminal alkenyl group or terminal alkynyl group, all of B3, C3, B4 and C4 are unsubstituted, q is 1, n is 1, p is 1, and s is 0.

Optionally C1 and C2 are both substituted at the α carbon with a terminal alkenyl group or terminal alkynyl group, all of B3, C3, B4 and C4 are unsubstituted, q is 1, p is 1, m is 1, and s is 0.

Optionally C1 and C2 are both substituted at the α carbon with a terminal alkenyl group or terminal alkynyl group, all of B3, C3, B4 and C4 are unsubstituted, n is 1, p is 1, m is 1, and s is 0.

Optionally C1 and C2 are both substituted at the α carbon with a terminal alkenyl group or terminal alkynyl group, all of B3, C3, B4 and C4 are unsubstituted, r is 0, q is 1, n is 1, p is 1 and s is 0.

Optionally C1 and C2 are both substituted at the α carbon with a terminal alkenyl group or terminal alkynyl group, all of B3, C3, B4 and C4 are unsubstituted, r is 0, q is 1, n is 1, m is 1 and s is 0.

Optionally C1 and C2 are both substituted at the α carbon with a terminal alkenyl group or terminal alkynyl group, all of B3, C3, B4 and C4 are unsubstituted, r is 0, q is 1, p is 1, m is 1 and s is 0.

Optionally C1 and C2 are both substituted at the α carbon with a terminal alkenyl group or terminal alkynyl group, all of B3, C3, B4 and C4 are unsubstituted, r is 0, n is 1, p is 1, m is 1 and s is 0.

Optionally C1 and C2 are both substituted at the α carbon with a terminal alkenyl group or terminal alkynyl group, all of B3, C3, B4 and C4 are unsubstituted, q is 1, n is 1, p is 1, m is 1 and s is 0.

Optionally C1 and C2 are both substituted at the α carbon with a terminal alkenyl group or terminal alkynyl group, all of B3, C3, B4 and C4 are unsubstituted, r is 0, s is 0, q is 1, n is 1, p is 1 and m is 1.

Optionally B6 and/or C6 are unsubstituted.

Optionally C1 and C2 are both substituted at the α carbon with a terminal alkenyl group or terminal alkynyl group, all of B3, C3, B4, C4, B6 and C6 are unsubstituted and r is 0.

Optionally C1 and C2 are both substituted at the α carbon with a terminal alkenyl group or terminal alkynyl group, all of B3, C3, B4, C4, B6 and C6 are unsubstituted and q is 1.

Optionally C1 and C2 are both substituted at the α carbon with a terminal alkenyl group or terminal alkynyl group, all of B3, C3, B4, C4, B6 and C6 are unsubstituted and n is 1.

Optionally C1 and C2 are both substituted at the α carbon with a terminal alkenyl group or terminal alkynyl group, all of B3, C3, B4, C4, B6 and C6 are unsubstituted and p is 1.

Optionally C1 and C2 are both substituted at the α carbon with a terminal alkenyl group or terminal alkynyl group, all of B3, C3, B4, C4, B6 and C6 are unsubstituted and m is 1.

Optionally C1 and C2 are both substituted at the α carbon with a terminal alkenyl group or terminal alkynyl group, all of B3, C3, B4, C4, B6 and C6 are unsubstituted and s is 0.

Optionally C1 and C2 are both substituted at the α carbon with a terminal alkenyl group or terminal alkynyl group, all of B3, C3, B4, C4, B6 and C6 are unsubstituted, q is 1 and n is 1.

Optionally C1 and C2 are both substituted at the α carbon with a terminal alkenyl group or terminal alkynyl group, all of B3, C3, B4, C4, B6 and C6 are unsubstituted, q is 1 and p is 1.

Optionally C1 and C2 are both substituted at the α carbon with a terminal alkenyl group or terminal alkynyl group, all of B3, C3, B4, C4, B6 and C6 are unsubstituted, q is 1 and m is 1.

Optionally C1 and C2 are both substituted at the α carbon with a terminal alkenyl group or terminal alkynyl group, all of B3, C3, B4, C4, B6 and C6 are unsubstituted, n is 1 and p is 1.

Optionally C1 and C2 are both substituted at the α carbon with a terminal alkenyl group or terminal alkynyl group, all of B3, C3, B4, C4, B6 and C6 are unsubstituted, n is 1 and m is 1.

Optionally C1 and C2 are both substituted at the α carbon with a terminal alkenyl group or terminal alkynyl group, all of B3, C3, B4, C4, B6 and C6 are unsubstituted, p is 1 and m is 1.

Optionally C1 and C2 are both substituted at the α carbon with a terminal alkenyl group or terminal alkynyl group, all of B3, C3, B4, C4, B6 and C6 are unsubstituted, r is 0 and q is 1.

Optionally C1 and C2 are both substituted at the α carbon with a terminal alkenyl group or terminal alkynyl group, all of B3, C3, B4, C4, B6 and C6 are unsubstituted, r is 0 and n is 1.

Optionally C1 and C2 are both substituted at the α carbon with a terminal alkenyl group or terminal alkynyl group, all of B3, C3, B4, C4, B6 and C6 are unsubstituted, r is 0 and p is 1.

Optionally C1 and C2 are both substituted at the α carbon with a terminal alkenyl group or terminal alkynyl group, all of B3, C3, B4, C4, B6 and C6 are unsubstituted, r is 0 and m is 1.

Optionally C1 and C2 are both substituted at the α carbon with a terminal alkenyl group or terminal alkynyl group, all of B3, C3, B4, C4, B6 and C6 are unsubstituted, s is 0 and q is 1.

Optionally C1 and C2 are both substituted at the α carbon with a terminal alkenyl group or terminal alkynyl group, all of B3, C3, B4, C4, B6 and C6 are unsubstituted, s is 0 and n is 1.

Optionally C1 and C2 are both substituted at the α carbon with a terminal alkenyl group or terminal alkynyl group, all of B3, C3, B4, C4, B6 and C6 are unsubstituted, s is 0 and p is 1.

Optionally C1 and C2 are both substituted at the α carbon with a terminal alkenyl group or terminal alkynyl group, all of B3, C3, B4, C4, B6 and C6 are unsubstituted, s is 0 and m is 1.

Optionally C1 and C2 are both substituted at the α carbon with a terminal alkenyl group or terminal alkynyl group, all of B3, C3, B4, C4, B6 and C6 are unsubstituted, r is 0 and s is 0.

Optionally C1 and C2 are both substituted at the α carbon with a terminal alkenyl group or terminal alkynyl group, all of B3, C3, B4, C4, B6 and C6 are unsubstituted, q is 1, n is 1 and p is 1.

Optionally C1 and C2 are both substituted at the α carbon with a terminal alkenyl group or terminal alkynyl group, all of B3, C3, B4, C4, B6 and C6 are unsubstituted, q is 1, n is 1 and m is 1.

Optionally C1 and C2 are both substituted at the α carbon with a terminal alkenyl group or terminal alkynyl group, all of B3, C3, B4, C4, B6 and C6 are unsubstituted, q is 1, p is 1 and m is 1.

Optionally C1 and C2 are both substituted at the α carbon with a terminal alkenyl group or terminal alkynyl group, all of B3, C3, B4, C4, B6 and C6 are unsubstituted, n is 1, p is 1 and m is 1.

Optionally C1 and C2 are both substituted at the α carbon with a terminal alkenyl group or terminal alkynyl group, all of B3, C3, B4, C4, B6 and C6 are unsubstituted, r is 0, q is 1 and n is 1.

Optionally C1 and C2 are both substituted at the α carbon with a terminal alkenyl group or terminal alkynyl group, all of B3, C3, B4, C4, B6 and C6 are unsubstituted, r is 0, q is 1 and p is 1.

Optionally C1 and C2 are both substituted at the α carbon with a terminal alkenyl group or terminal alkynyl group, all of B3, C3, B4, C4, B6 and C6 are unsubstituted, r is 0, q is 1 and m is 1.

Optionally C1 and C2 are both substituted at the α carbon with a terminal alkenyl group or terminal alkynyl group, all of B3, C3, B4, C4, B6 and C6 are unsubstituted, r is 0, q is 1 and s is 0.

Optionally C1 and C2 are both substituted at the α carbon with a terminal alkenyl group or terminal alkynyl group, all of B3, C3, B4, C4, B6 and C6 are unsubstituted, r is 0, n is 1 and p is 1.

Optionally C1 and C2 are both substituted at the α carbon with a terminal alkenyl group or terminal alkynyl group, all of B3, C3, B4, C4, B6 and C6 are unsubstituted, r is 0, n is 1 and m is 1.

Optionally C1 and C2 are both substituted at the α carbon with a terminal alkenyl group or terminal alkynyl group, all of B3, C3, B4, C4, B6 and C6 are unsubstituted, r is 0, n is 1 and s is 0.

Optionally C1 and C2 are both substituted at the α carbon with a terminal alkenyl group or terminal alkynyl group, all of B B3, C3, B4, C4, B6 and C6 are unsubstituted, r is 0, p is 1 and m is 1.

Optionally C1 and C2 are both substituted at the α carbon with a terminal alkenyl group or terminal alkynyl group, all of B3, C3, B4, C4, B6 and C6 are unsubstituted, r is 0, p is 1 and s is 0.

Optionally C1 and C2 are both substituted at the α carbon with a terminal alkenyl group or terminal alkynyl group, all of B3, C3, B4, C4, B6 and C6 are unsubstituted, r is 0, m is 1 and s is 0.

Optionally C1 and C2 are both substituted at the α carbon with a terminal alkenyl group or terminal alkynyl group, all of B3, C3, B4, C4, B6 and C6 are unsubstituted, r is 0, q is 1, n is 1, and p is 1.

Optionally C1 and C2 are both substituted at the α carbon with a terminal alkenyl group or terminal alkynyl group, all of B3, C3, B4, C4, B6 and C6 are unsubstituted, r is 0, q is 1, n is 1, and m is 1.

Optionally C1 and C2 are both substituted at the α carbon with a terminal alkenyl group or terminal alkynyl group, all of B3, C3, B4, C4, B6 and C6 are unsubstituted, r is 0, q is 1, n is 1, and m is 1.

Optionally C1 and C2 are both substituted at the α carbon with a terminal alkenyl group or terminal alkynyl group, all of B3, C3, B4, C4, B6 and C6 are unsubstituted, r is 0, q is 1, n is 1, and s is 0.

Optionally C1 and C2 are both substituted at the α carbon with a terminal alkenyl group or terminal alkynyl group, all of B3, C3, B4, C4, B6 and C6 are unsubstituted, r is 0, q is 1, p is 1, and m is 1.

Optionally C1 and C2 are both substituted at the α carbon with a terminal alkenyl group or terminal alkynyl group, all of B3, C3, B4, C4, B6 and C6 are unsubstituted, r is 0, q is 1, p is 1, and s is 0.

Optionally C1 and C2 are both substituted at the α carbon with a terminal alkenyl group or terminal alkynyl group, all of B3, C3, B4, C4, B6 and C6 are unsubstituted, r is 0, q is 1, m is 1, and s is 0.

Optionally C1 and C2 are both substituted at the α carbon with a terminal alkenyl group or terminal alkynyl group, all of B3, C3, B4, C4, B6 and C6 are unsubstituted, r is 0, n is 1, p is 1, and m is 1.

Optionally C1 and C2 are both substituted at the α carbon with a terminal alkenyl group or terminal alkynyl group, all of B3, C3, B4, C4, B6 and C6 are unsubstituted, r is 0, n is 1, p is 1, and s is 0.

Optionally C1 and C2 are both substituted at the α carbon with a terminal alkenyl group or terminal alkynyl group, all of B3, C3, B4, C4, B6 and C6 are unsubstituted, r is 0, n is 1, m is 1, and s is 0.

Optionally C1 and C2 are both substituted at the α carbon with a terminal alkenyl group or terminal alkynyl group, all of B3, C3, B4, C4, B6 and C6 are unsubstituted, r is 0, p is 1, m is 1, and s is 0.

Optionally C1 and C2 are both substituted at the α carbon with a terminal alkenyl group or terminal alkynyl group, all of B3, C3, B4, C4, B6 and C6 are unsubstituted, q is 1, n is 1, p is 1, and m is 1.

Optionally C1 and C2 are both substituted at the α carbon with a terminal alkenyl group or terminal alkynyl group, all of B3, C3, B4, C4, B6 and C6 are unsubstituted, q is 1, n is 1, p is 1, and s is 0.

Optionally C1 and C2 are both substituted at the α carbon with a terminal alkenyl group or terminal alkynyl group, all of B3, C3, B4, C4, B6 and C6 are unsubstituted, q is 1, p is 1, m is 1, and s is 0.

Optionally C1 and C2 are both substituted at the α carbon with a terminal alkenyl group or terminal alkynyl group, all of B3, C3, B4, C4, B6 and C6 are unsubstituted, n is 1, p is 1, m is 1, and s is 0.

Optionally C1 and C2 are both substituted at the α carbon with a terminal alkenyl group or terminal alkynyl group, all of B3, C3, B4, C4, B6 and C6 are unsubstituted, r is 0, q is 1, n is 1, p is 1 and m is 1.

Optionally C1 and C2 are both substituted at the α carbon with a terminal alkenyl group or terminal alkynyl group, all of B3, C3, B4, C4, B6 and C6 are unsubstituted, r is 0, q is 1, n is 1, p is 1 and s is 0.

Optionally C1 and C2 are both substituted at the α carbon with a terminal alkenyl group or terminal alkynyl group, all of B3, C3, B4, C4, B6 and C6 are unsubstituted, r is 0, q is 1, n is 1, m is 1 and s is 0.

Optionally C1 and C2 are both substituted at the α carbon with a terminal alkenyl group or terminal alkynyl group, all of B3, C3, B4, C4, B6 and C6 are unsubstituted, r is 0, q is 1, p is 1, m is 1 and s is 0.

Optionally C1 and C2 are both substituted at the α carbon with a terminal alkenyl group or terminal alkynyl group, all of B3, C3, B4, C4, B6 and C6 are unsubstituted, r is 0, n is 1, p is 1, m is 1 and s is 0.

Optionally C1 and C2 are both substituted at the α carbon with a terminal alkenyl group or terminal alkynyl group, all of B3, C3, B4, C4, B6 and C6 are unsubstituted, q is 1, n is 1, p is 1, m is 1 and s is 0.

Optionally C1 and C2 are both substituted at the α carbon with a terminal alkenyl group or terminal alkynyl group, all of B3, C3, B4, C4, B6 and C6 are unsubstituted, r is 0, s is 0, q is 1, n is 1, p is 1 and m is 1.

Optionally C6 is leucine, isoleucine or valine. Optionally B6 is leucine.

Optionally both B3 and C6 are each independently selected from leucine, isoleucine or valine. Optionally B3 and C6 are each leucine.

Optionally both C3 and C6 are each independently selected from leucine, isoleucine or valine. Optionally C3 and C6 are each leucine.

Optionally both B4 and C6 are each independently selected from leucine, isoleucine or valine. Optionally B4 and C6 are each leucine.

Optionally both B5 and C6 are each independently selected from leucine, isoleucine or valine. Optionally B5 and C6 are each leucine.

Optionally all of B3, C3 and C6 are each independently selected from leucine, isoleucine or valine. Optionally all of B3, C3 and C6 are leucine.

Optionally all of B3, B4 and C6 are each independently selected from leucine, isoleucine or valine. Optionally all of B3, B4 and C6 are leucine.

Optionally all of B3, B5 and C6 are each independently selected from leucine, isoleucine or valine. Optionally all of B3, B5 and C6 are leucine.

Optionally all of C3, B4 and C6 are each independently selected from leucine, isoleucine or valine. Optionally all of C3, B4 and C6 are leucine.

Optionally all of C3, B5 and C6 are each independently selected from leucine, isoleucine or valine. Optionally all of C3, B5 and C6 are leucine.

Optionally all of B4, B5 and C6 are each independently selected from leucine, isoleucine or valine. Optionally all of B4, B5 and C6 are leucine.

Optionally all of B3, C3, B4, and C6 are each independently selected from leucine, isoleucine or valine. Optionally all of B3, C3, B4, and C6 are leucine.

Optionally all of B3, C3, B5, and C6 are each independently selected from leucine, isoleucine or valine. Optionally all of B3, C3, B5, and C6 are leucine.

Optionally all of B3, B4, B5, and C6 are each independently selected from leucine, isoleucine or valine. Optionally all of B3, B4, B5, and C6 are leucine.

Optionally all of C3, B4, B5, and C6 are each independently selected from leucine, isoleucine or valine. Optionally all of C3, B4, B5, and C6 are leucine.

Optionally all of B3, C3, B4, B5, and C6 are each independently selected from leucine, isoleucine or valine. Optionally all of B3, C3, B4, B5, and C6 are leucine.

Optionally both B3 and C3 are independently selected from leucine, isoleucine or valine (optionally leucine) and A6 is lysine or arginine (optionally lysine).

Optionally both B3 and C3 are independently selected from leucine, isoleucine or valine (optionally leucine) and both A1 and A6 are independently selected from lysine or arginine (optionally lysine).

Optionally both B3 and C3 are independently selected from leucine, isoleucine or valine (optionally leucine) and both A2 and A6 are independently selected from lysine or arginine (optionally lysine).

Optionally both B3 and C3 are independently selected from leucine, isoleucine or valine (optionally leucine) and both A3 and A6 are independently selected from lysine or arginine (optionally lysine).

Optionally both B3 and C3 are independently selected from leucine, isoleucine or valine (optionally leucine) and both A4 and A6 are independently selected from lysine or arginine (optionally lysine).

Optionally both B3 and C3 are independently selected from leucine, isoleucine or valine (optionally leucine) and A1, A2 and A6 are each independently selected from lysine or arginine (optionally lysine).

Optionally both B3 and C3 are independently selected from leucine, isoleucine or valine (optionally leucine) and A1, A3 and A6 are each independently selected from lysine or arginine (optionally lysine).

Optionally both B3 and C3 are independently selected from leucine, isoleucine or valine (optionally leucine) and A1, A4 and A6 are each independently selected from lysine or arginine (optionally lysine).

Optionally both B3 and C3 are independently selected from leucine, isoleucine or valine (optionally leucine) and A2, A3 and A6 are each independently selected from lysine or arginine (optionally lysine).

Optionally both B3 and C3 are independently selected from leucine, isoleucine or valine (optionally leucine) and A2, A4 and A6 are each independently selected from lysine or arginine (optionally lysine).

Optionally both B3 and C3 are independently selected from leucine, isoleucine or valine (optionally leucine) and A3, A4 and A6 are each independently selected from lysine or arginine (optionally lysine).

Optionally both B3 and C3 are independently selected from leucine, isoleucine or valine (optionally leucine) and A1, A2, A3 and A6 are each independently selected from lysine or arginine (optionally lysine).

Optionally both B3 and C3 are independently selected from leucine, isoleucine or valine (optionally leucine) and A1, A2, A4 and A6 are each independently selected from lysine or arginine (optionally lysine).

Optionally both B3 and C3 are independently selected from leucine, isoleucine or valine (optionally leucine) and A1, A3, A4 and A6 are each independently selected from lysine or arginine (optionally lysine).

Optionally both B3 and C3 are independently selected from leucine, isoleucine or valine (optionally leucine) and A2, A3, A4 and A6 are each independently selected from lysine or arginine (optionally lysine).

Optionally both B3 and C3 are independently selected from leucine, isoleucine or valine (optionally leucine) and A1, A2, A3, A4 and A6 are each independently selected from lysine or arginine (optionally lysine).

Optionally both B3 and C3 are independently selected from leucine, isoleucine or valine (optionally leucine) and A1, A2, A3, A4 and A6 are each independently selected from lysine or arginine (optionally lysine).

Optionally all of B3, C3, B4, B5, and C6 are each independently selected from leucine, isoleucine or valine and A1, A2, A3, A4 and A6 are each independently selected from lysine or arginine (optionally lysine).

Optionally both B3 and C3 are independently selected from leucine, isoleucine or valine (optionally leucine) and each of A1, A2, A3 is independently selected from lysine or arginine (optionally lysine) and each of m, n and p, q are both 1 and r and s are each 0.

Optionally both B3 and C3 are independently selected from leucine, isoleucine or valine (optionally leucine) and each of A1, A2, A3, A4 and A6 is lysine or arginine (optionally lysine) and n is 1.

Optionally both B3 and C3 are leucine, isoleucine or valine (optionally leucine) and each of A1, A2, A3, A4, and A6 is lysine or arginine (optionally lysine) and p is 1.

Optionally both B3 and C3 are leucine, isoleucine or valine (optionally leucine) and each of A1, A2, A3, A4, and A6 is lysine or arginine (optionally lysine) and m is 1.

Optionally both B3 and C3 are leucine, isoleucine or valine (optionally leucine) and each of A1, A2, A3, A4, and A6 is lysine or arginine (optionally lysine) and n and p are 1.

Optionally both B3 and C3 are leucine, isoleucine or valine (optionally leucine) and each of A1, A2, A3, A4, and A6 is lysine or arginine (optionally lysine) and n and m are both 1.

Optionally both B3 and C3 are leucine, isoleucine or valine (optionally leucine) and each of A1, A2, A3, A4, and A6 is lysine or arginine (optionally lysine) and m and p are both 1.

Optionally both B3 and C3 are leucine, isoleucine or valine (optionally leucine) and each of A1, A2, A3, A4, and A6 is lysine or arginine (optionally lysine) and each of m, n and p are both 1.

Optionally both B3 and C3 are leucine, isoleucine or valine (optionally leucine) and each of A1, A2, A3, A4, and A6 is lysine or arginine (optionally lysine) and each of m, n, p and q are both 1.

Optionally both B3 and C3 are leucine, isoleucine or valine (optionally leucine) and each of A1, A2, A3, A4, and A6 is lysine or arginine (optionally lysine) and each of m, n, p and q are both 1 and r and s are each 0.

Optionally either:

i) C6, B5, B4, B3 and C3 are each independently selected from leucine, isoleucine and valine (and are optionally each leucine), each of A1, A2, A3, A4 and A6 is independently selected from lysine and arginine (and are optionally each lysine), each of m, n, p and q are 1, r and s are 0, 01 and C2 are both substituted at the α carbon with a terminal alkenyl group or terminal alkynyl group, B3, C3, B4, C4, B5, B6, C5 and C6 are all unsubstituted, and each of B1, C1, D1, B2, C2, D2, C4, C5 and B6 are each independently selected from valine, alanine, isoleucine and leucine (and are optionally each alanine); or

ii) C6, B5, B4, B3 and C3 are each independently selected from leucine, isoleucine or valine (and are optionally each leucine), each of A1, A2, A3, A4 and A6 is independently selected from lysine or arginine (and are optionally each lysine), each of m, n, p and q are 1, r and s are 0, C1, C2 and C5 are each substituted at the α carbon with a terminal alkenyl group or terminal alkynyl group, B3, C3, B4, C4, B5, B6, and C6 are all unsubstituted, and each of B1, C1, D1, B2, C2, D2, C4, C5 and B6 are each independently selected from valine, alanine, isoleucine and leucine (and are optionally each alanine).

In a particular embodiment, the peptide has the following sequence:
B5-C5-(D5)ᵣ-A4-B4-(C4)_{q}-A1-B1-C1-(D1)ₙ-A3-B3-(C3)ₚ-A2-B2-C2-(D2)ₘ-A6-B6-C6-(D6)ₛ, wherein:
A1 is an amino acid that has a positively charged side chain at pH 7;
B1 is an amino acid having a hydrophobic side chain;
C1 is an amino acid having a hydrophobic side chain;
D1, when present, is an amino acid having a hydrophobic side chain; and
n is 0 or 1;
wherein one of B1, C1, or D1 is substituted at the α carbon with a terminal alkenyl group or terminal alkynyl group;
A2 is an amino acid that has a positively charged side chain at pH 7;
B2 is an amino acid having a hydrophobic side chain;
C2 is an amino acid having a hydrophobic side chain;
D2, when present, is an amino acid having a hydrophobic side chain; and
m is 0 or 1;
wherein at least one of B2, C2, or D2 is substituted at the α carbon with a terminal alkenyl group or terminal alkynyl group;
A3 is an amino acid that has a positively charged side chain at pH 7;
B3 is an amino acid having a hydrophobic side chain;
C3, when present, is an amino acid having a hydrophobic side chain; and
p is 0 or 1;
wherein B3 and C3 are independently (i) unsubstituted or (ii) substituted at the α carbon with a terminal alkenyl group or terminal alkynyl group;
A4 is an amino acid that has a positively charged side chain at pH 7;
B4 is an amino acid having a hydrophobic side chain;
C4, when present, is an amino acid having a hydrophobic side chain; and
q is 0 or 1;
wherein B4 and C4 are independently (i) unsubstituted or (ii) substituted at the α carbon with a terminal alkenyl group or terminal alkynyl group;
B5 is an amino acid having a hydrophobic side chain;
C5 is an amino acid having a hydrophobic side chain;
D5, when present, is an amino acid having a hydrophobic side chain; and
r is 0 or 1;

wherein B5 and C5 are independently (i) unsubstituted or (ii) substituted at the α carbon with a terminal alkenyl group or terminal alkynyl group; and
A6 is an amino acid that has a positively charged side chain at pH 7;
B6 is an amino acid having a hydrophobic side chain;
C6 is an amino acid having a hydrophobic side chain;
D6, when present, is an amino acid having a hydrophobic side chain; and
s is 0 or 1;
wherein B6 and C6 are independently (i) unsubstituted or (ii) substituted at the α carbon with a terminal alkenyl group or terminal alkynyl group.

Any of the peptides of the invention may further comprise a seventh sequence [A7-B7-C7-(D7)ₜ]ᵤ, and/or an eighth sequence [A8-B8-C8-(D8)ₓ]_{y}, wherein:
A7 is an amino acid that has a positively charged side chain at pH 7;
B7 is an amino acid having a hydrophobic side chain;
C7 is an amino acid having a hydrophobic side chain;
D7, when present, is an amino acid having a hydrophobic side chain;
t is 0 or 1; and
u is 0 to 6;
A8 is an amino acid that has a positively charged side chain at pH 7;
B8 is an amino acid having a hydrophobic side chain;
C8 is an amino acid having a hydrophobic side chain;
D8, when present, is an amino acid having a hydrophobic side chain;
x is 0 or 1; and
y is 0 to 6.

Optionally the or each A7 is independently selected from lysine or arginine (optionally each A7 is lysine).

Optionally the or each A8 is independently selected from lysine or arginine (optionally each A8 is lysine).

Optionally the or each A7 is independently selected from lysine or arginine (optionally each A7 is lysine) and the or each A8 is independently selected from lysine or arginine (optionally each A8 is lysine).

Optionally the or each B7 is independently selected from glycine, valine, alanine, isoleucine, leucine, proline and methionine. For example, the or each B7 is selected from lysine and alanine.

Optionally the or each B7 is independently selected from glycine, valine, alanine, isoleucine, leucine, proline and methionine (for example, the or each B7 is selected from lysine) and alanine and the or each A7 is independently selected from lysine or arginine (optionally each A7 is lysine).

Optionally the or each B7 is independently selected from glycine, valine, alanine, isoleucine, leucine, proline and methionine (for example, the or each B7 is selected from lysine and alanine) and the or each A8 is independently selected from lysine or arginine (optionally each A8 is lysine).

Optionally the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine)

Optionally the or each B8 is independently selected from glycine, valine, alanine, isoleucine, leucine, proline and methionine. For example, the or each B8 is selected from lysine and alanine.

Optionally the or each B8 is independently selected from glycine, valine, alanine, isoleucine, leucine, proline and methionine (for example, the or each B8 is selected from lysine) and alanine and the or each A7 is independently selected from lysine or arginine (optionally each A7 is lysine).

Optionally the or each B8 is independently selected from glycine, valine, alanine, isoleucine, leucine, proline and methionine (for example, the or each B8 is selected from lysine and alanine) and the or each A8 is independently selected from lysine or arginine (optionally each A8 is lysine).

Optionally the or each B8 is independently selected from glycine, valine, alanine, isoleucine, leucine, proline and methionine (for example, the or each B8 is selected from lysine and alanine) and the or each A7 is independently selected from lysine or arginine (optionally each A7 is lysine) and the or each A8 is independently selected from lysine or arginine (optionally each A8 is lysine).

Optionally the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine).

Optionally the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine).

Optionally the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A8 is independently selected from lysine or arginine (optionally each A8 is lysine).

Optionally the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine), the or each A7 is independently selected from lysine or arginine (optionally each A7 is lysine) and the or each A8 is independently selected from lysine or arginine (optionally each A8 is lysine).

Optionally the or each C7 is independently selected from glycine, valine, alanine, isoleucine, leucine, proline and methionine. For example, the or each C7 is selected from lysine and alanine.

Optionally the or each C8 is independently selected from glycine, valine, alanine, isoleucine, leucine, proline and methionine. For example, the or each C8 is selected from lysine and alanine.

Optionally the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine. For example, the or each C8 is selected from lysine and alanine.

Optionally the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine), the or each A7 is independently selected from lysine or arginine (optionally each A7 is lysine) and the or each A8 is independently selected from lysine or arginine (optionally each A8 is lysine).

Optionally the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine).

Optionally the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine), and the or each A7 is independently selected from lysine or arginine (optionally each A7 is lysine) and the or each A8 is independently selected from lysine or arginine (optionally each A8 is lysine).

Optionally the or each D7 (when present) is independently selected from glycine, valine, alanine, isoleucine, leucine, proline and methionine. For example, the or each D7 (when present) is selected from lysine and alanine.

Optionally the or each D8 (when present) is independently selected from glycine, valine, alanine, isoleucine, leucine, proline and methionine. For example, the or each D8 (when present) is selected from lysine and alanine.

Optionally the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine).

Optionally the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine), the or each A7 is independently selected from lysine or arginine (optionally each A7 is lysine) and the or each A8 is independently selected from lysine or arginine (optionally each A8 is lysine).

Optionally the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine).

Optionally the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine), and the or each A7 is independently selected from lysine or arginine (optionally each A7 is lysine) and the or each A8 is independently selected from lysine or arginine (optionally each A8 is lysine).

Optionally the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine. For example, the or each C8 is selected from lysine and alanine.

Optionally the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each A7 is independently selected from lysine or arginine (optionally each A7 is lysine) and the or each A8 is independently selected from lysine or arginine (optionally each A8 is lysine).

Optionally the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine).

Optionally the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

Each t is independently selected from 0 and 1.

u can be 0, 1, 2, 3, 4, 5 or 6.

For example, u is 0 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 0 and the or each D8 (when present) is independently selected from glycine, valine, alanine, isoleucine, leucine, proline and methionine (for example, the or each D8 (when present) is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each A8 is independently selected from lysine or arginine (optionally each A8 is lysine).

For example, u is 1 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 2 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 3 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 4 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 5 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 6 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

Each x is independently selected from 0 and 1.

y can be 0, 1, 2, 3, 4, 5 or 6.

For example, y is 0 and the or each D7 (when present) is independently selected from glycine, valine, alanine, isoleucine, leucine, proline and methionine (for example, the or each D7 (when present) is selected from lysine and alanine) and the or each C7 is independently selected from glycine, valine, alanine, isoleucine, leucine, proline and methionine (for example, the or each C7 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine).

For example, y is 1 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, y is 2 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, y is 3 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, y is 4 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, y is 5 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, y is 6 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, both u and y are 0.

For example, u is 1 and y is 1 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 1 and y is 2 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 1 and y is 3 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 1 and y is 4 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 1 and y is 5 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 1 and y is 6 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 2 and y is 1 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 2 and y is 2 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 2 and y is 3 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 2 and y is 4 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 2 and y is 5 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 2 and y is 6 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 3 and y is 1 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 3 and y is 2 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 3 and y is 3 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 3 and y is 4 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 3 and y is 5 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 3 and y is 6 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 4 and y is 1 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 4 and y is 2 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 4 and y is 3 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 4 and y is 4 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 4 and y is 5 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 4 and y is 6 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 5 and y is 1 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 5 and y is 2 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 5 and y is 3 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 5 and y is 4 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 5 and y is 5 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 5 and y is 6 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 6 and y is 1 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 6 and y is 2 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 6 and y is 3 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 6 and y is 4 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 6 and y is 5 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 6 and y is 6 and the or each D7 (when present) is independently selected from glycine, valine, alanine, isoleucine, leucine, proline and methionine (for example, the or each D7 (when present) is selected from lysine and alanine) and the or each D8 (when present) is independently selected from glycine, valine, alanine, isoleucine, leucine, proline and methionine (for example, the or each D8 (when present) is selected from lysine and alanine) and the or each C7 is independently selected from glycine, valine, alanine, isoleucine, leucine, proline and methionine (for example, the or each C7 is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine) and the or each A8 is independently selected from lysine or arginine (optionally each A8 is lysine).

For example, u is 0 and y is 1 and the or each D8 (when present) is independently selected from glycine, valine, alanine, isoleucine, leucine, proline and methionine (for example, the or each D8 (when present) is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each A8 is independently selected from lysine or arginine (optionally each A8 is lysine).

For example, u is 0 and y is 2 and the or each D8 (when present) is independently selected from glycine, valine, alanine, isoleucine, leucine, proline and methionine (for example, the or each D8 (when present) is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each A8 is independently selected from lysine or arginine (optionally each A8 is lysine).

For example, u is 0 and y is 3 and the or each D8 (when present) is independently selected from glycine, valine, alanine, isoleucine, leucine, proline and methionine (for example, the or each D8 (when present) is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each A8 is independently selected from lysine or arginine (optionally each A8 is lysine).

For example, u is 0 and y is 4 and the or each D8 (when present) is independently selected from glycine, valine, alanine, isoleucine, leucine, proline and methionine (for example, the or each D8 (when present) is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each A8 is independently selected from lysine or arginine (optionally each A8 is lysine).

For example, u is 0 and y is 5 and the or each D8 (when present) is independently selected from glycine, valine, alanine, isoleucine, leucine, proline and methionine (for example, the or each D8 (when present) is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each A8 is independently selected from lysine or arginine (optionally each A8 is lysine).

For example, u is 0 and y is 6 and the or each D8 (when present) is independently selected from glycine, valine, alanine, isoleucine, leucine, proline and methionine (for example, the or each D8 (when present) is selected from lysine and alanine) and the or each C8 is independently selected from glycine, valine, alanine, isoleucine, leucine, proline and methionine (for example, the or each C8 is selected from lysine and alanine) and the or each B8 is independently selected from glycine, valine, alanine, isoleucine, leucine, proline and methionine (for example, the or each B8 is selected from lysine and alanine) and the or each A8 is independently selected from lysine or arginine (optionally each A8 is lysine).

For example, u is 1 and y is 0 and the or each D7 (when present) is independently selected from glycine, valine, alanine, isoleucine, leucine, proline and methionine (for example, the or each D7 (when present) is selected from lysine and alanine) and the or each C7 is independently selected from glycine, valine, alanine, isoleucine, leucine, proline and methionine (for example, the or each C7 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine).

For example, u is 2 and y is 0 and the or each D7 (when present) is independently selected from glycine, valine, alanine, isoleucine, leucine, proline and methionine (for example, the or each D7 (when present) is selected from lysine and alanine) and the or each C7 is independently selected from glycine, valine, alanine, isoleucine, leucine, proline and methionine (for example, the or each C7 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine).

For example, u is 3 and y is 0 and the or each D7 (when present) is independently selected from glycine, valine, alanine, isoleucine, leucine, proline and methionine (for example, the or each D7 (when present) is selected from lysine and alanine) and the or each C7 is independently selected from glycine, valine, alanine, isoleucine, leucine, proline and methionine (for example, the or each C7 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine).

For example, u is 4 and y is 0 and the or each D7 (when present) is independently selected from glycine, valine, alanine, isoleucine, leucine, proline and methionine (for example, the or each D7 (when present) is selected from lysine and alanine) and the or each C7 is independently selected from glycine, valine, alanine, isoleucine, leucine, proline and methionine (for example, the or each C7 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine).

For example, u is 5 and y is 0 and the or each D7 (when present) is independently selected from glycine, valine, alanine, isoleucine, leucine, proline and methionine (for example, the or each D7 (when present) is selected from lysine and alanine) and the or each C7 is independently selected from glycine, valine, alanine, isoleucine, leucine, proline and methionine (for example, the or each C7 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine).

For example, u is 6 and y is 0 and the or each D7 (when present) is independently selected from glycine, valine, alanine, isoleucine, leucine, proline and methionine (for example, the or each D7 (when present) is selected from lysine and alanine) and the or each C7 is independently selected from glycine, valine, alanine, isoleucine, leucine, proline and methionine (for example, the or each C7 is selected from lysine and alanine) and the or each B7 is independently selected from glycine, valine, alanine, isoleucine, leucine, proline and methionine (for example, the or each B7 is selected from lysine and alanine) and the or each A7 is independently selected from lysine or arginine (optionally each A7 is lysine).

A peptide of the invention optionally has the following sequence:
[A7-B7-C7-(D7)ₜ]ᵤ-B5-C5-(D5)ᵣ-A4-B4-(C4)_{q}-A1-B1-C1-(D1)ₙ-A3-B3-(C3)ₚ-A2-B2-C2-(D2)ₘ-A6-B6-C6(D6)ₛ-[A8-B8-C8-(D8)ₓ]_{y},
wherein:
A1 is an amino acid that has a positively charged side chain at pH 7;
B1 is an amino acid having a hydrophobic side chain;
C1 is an amino acid having a hydrophobic side chain;
D1, when present, is an amino acid having a hydrophobic side chain; and
n is 0 or 1;
wherein one of B1, C1, or D1 is substituted at the α carbon with a terminal alkenyl group or terminal alkynyl group;
A2 is an amino acid that has a positively charged side chain at pH 7;
B2 is an amino acid having a hydrophobic side chain;
C2 is an amino acid having a hydrophobic side chain;
D2, when present, is an amino acid having a hydrophobic side chain; and
m is 0 or 1;
wherein at least one of B2, C2, or D2 is substituted at the α carbon with a terminal alkenyl group or terminal alkynyl group;
A3 is an amino acid that has a positively charged side chain at pH 7;
B3 is an amino acid having a hydrophobic side chain;
C3, when present, is an amino acid having a hydrophobic side chain; and
p is 0 or 1;
wherein B3 and C3 are independently unsubstituted or substituted at the α carbon with a terminal alkenyl group or terminal alkynyl group;
A4 is an amino acid that has a positively charged side chain at pH 7;
B4 is an amino acid having a hydrophobic side chain;
C4, when present, is an amino acid having a hydrophobic side chain; and
q is 0 or 1;
wherein B4 and C4 are independently (i) unsubstituted or (ii) substituted at the α carbon with a terminal alkenyl group or terminal alkynyl group;
B5 is an amino acid having a hydrophobic side chain;
C5 is an amino acid having a hydrophobic side chain;
D5, when present, is an amino acid having a hydrophobic side chain; and
r is 0 or 1;
wherein B5 and C5 are independently (i) unsubstituted or (ii) substituted at the α carbon with a terminal alkenyl group or terminal alkynyl group;
A6 is an amino acid that has a positively charged side chain at pH 7;
B6 is an amino acid having a hydrophobic side chain;
C6 is an amino acid having a hydrophobic side chain;
D6, when present, is an amino acid having a hydrophobic side chain; and
s is 0 or 1;
wherein B6 and C6 are independently unsubstituted or substituted at the α carbon with a terminal alkenyl group or terminal alkynyl group;
A7 is an amino acid that has a positively charged side chain at pH 7;
B7 is an amino acid having a hydrophobic side chain;
C7 is an amino acid having a hydrophobic side chain;
D7, when present, is an amino acid having a hydrophobic side chain;
t is 0 or 1; and
u is 0 to 6; and
A8 is an amino acid that has a positively charged side chain at pH 7;
B8 is an amino acid having a hydrophobic side chain;
C8 is an amino acid having a hydrophobic side chain; and
D8, when present, is an amino acid having a hydrophobic side chain.

The peptide is optionally selected from the following group:

(SEQ ID NO: 1)
H-L A K L A K A A(R8) A K L L K A A(S5) AKA L-NH2
(also known as 246);
(SEQ ID NO: 2)
STY-L A K L A K A A(R8) A K L L K A A(S5) A K A
L-NH2_(246 Sty);
(SEQ ID NO: 1)
H-L A K L A K A A(R8) A K L L K A A(S5) A K A L-
NH2_(246 Sta);
(SEQ ID NO: 2)
STY-L A K L A K A A(R8) A K L L K A A(S5) A K A
L-NH2_(246 Sta Sty);
(SEQ ID NO: 3)
H-L A K L A K A A(R8) A K L L K A A(S8) A K A L-
NH2 (Oct815);
(SEQ ID NO: 4)
H-L A(R8) K L A K A A(R8) A K L L K A A(S8) A K A
L-NH2 (Oct2815);
and
(SEQ ID NO: 5)
H-L A(R8) K L A K A A A K L L K A A(S8) A K A L-
NH2 (Oct215);

wherein A(R8)=(R)-2-(7-octenyl)alanine; A(S8)=(S)-2-(7-octenyl)alanine; A(S5)=(S)-2-(4-pentenyl)alanine; and STY=N-Stearyl modification.

The peptides of the invention can be synthesised using a routine fluorenylmethyloxycarbonyl protecting group (F-moc) solid-phase peptide synthesis. The inclusion of the carbonyl modifications can be achieved by using specially adapted amino acids, which are commercially available. In this regard, the amino acid identified herein as A(R8) can be introduced into a peptide during synthesis using Fmoc-(R)-2-(7-octenyl)alanine-OH, A(S8) can be introduced into a peptide during synthesis using Fmoc-(S)-2-(7-octenyl)alanine-OH and A(S5) can be introduced into a peptide during synthesis using Fmoc-(S)-2-(4-pentenyl)alanine-OH. The synthetic products can be purified by reversed-phase high-performance liquid chromatography (RP-HPLC) and analysed by mass spectrometry (MS), as is standard in the art.

The peptides of the present invention can be combined with a cargo molecule. The cargo molecule may be essentially any type of drug cargo: a nucleic acid such as an RNA molecule, a DNA molecule or a mixmer, a protein or peptide, an RNP or an EV such as an exosome or a microvesicle (for instance an ARRM), or any other type of nanoparticle such as a virus, or essentially any other drug modality which can be complexed with and/or conjugated to the peptide. Specifically, the cargo to be delivered according to the present invention can be mRNA, antisense or splice-switching oligonucleotides, siRNA, shRNA, miRNA, plasmid DNA (pDNA), supercoiled or unsupercoiled plasmids, mini-circles, peptides, proteins, antibodies, antibody-drug conjugates, small molecule drugs, gene editing technology such as CRISPR-Cas9, TALENs, meganucleases, or vesicle-based cargos such as viruses (e.g. AAVs, lentiviruses, etc.). In one embodiment, the cargo may be a mixture of protein, nucleic acid and/or EVs. The cargo may, for instance, be a protein combined with a nucleic acid, an EV combined with a nucleic acid, a protein combined with an EV or a combination of all three. The EVs are preferably modified to comprise at least one drug of interest, for instance an mRNA, a protein, a peptide, an RNAi agent such as an siRNA, shRNA or miRNA, or a small molecule drug. In particularly preferred embodiments of the present invention, the EVs are exosomes modified to comprise a protein and/or an mRNA.

The cargo may be associated with or bound to the peptides of the present invention. The cargo may be associated with or bound to the peptides of the present invention by simply bringing the two into contact. The cargo may be bound by covalent or non-covalent interactions with the peptide. In preferred embodiments, the cargo is non-covalently complexed with the peptide, so as to create a nanoparticle-like structure. Such nanoparticle-like structures may have a hydrodynamic diameter (which is a suitable way of measuring the size of said nanoparticle-like structures) ranging from a few nanometers (nm) to a few micrometers (μm), typically 10 nm to 500 nm, preferably in the range of 20 nm to 300 nm.

In one embodiment, the nucleic acid cargo molecule transported by the peptide may be selected from the group comprising shRNA, siRNA, saRNA, miRNA, an anti-miRNA, mRNA, gRNA, pri-miRNA, pre-miRNA, circular RNA, piRNA, tRNA, rRNA, snRNA, lncRNA, ribozymes, mini-circle DNA, plasmid DNA, RNA/DNA vectors, trans-splicing oligonucleotides, splice-switching oligonucleotides, CRISPR guide strands, morpholinos (PMO) antisense oligonucleotides (ASO), peptide-nucleic acids (PNA), a viral genome and viral genetic material, but essentially any type of nucleic acid molecule can be delivered by the peptides of the present invention. Both single-stranded and double-stranded nucleic acid molecules are within the scope of the present invention, and the nucleic acid molecule may be naturally occurring (such as RNA or DNA) or may be a chemically synthesised RNA and/or DNA molecule which may comprise chemically modified nucleotides such as 2′-O-Me, 2′-O-Allyl, 2′-O-MOE, 2′-F, 2′-CE, 2′-EA 2′-FANA, LNA, CLNA, ENA, PNA, phosphorothioates, tricyclo-DNA, thionucleotides, phosphoramidate, PNA, PMO, etc.

When the nucleic acid cargo is an mRNA cargo molecule, the mRNA may be a naturally or non-naturally occurring mRNA. An mRNA may include one or more modified nucleobases, nucleosides, or nucleotides. A nucleobase of an mRNA is an organic base such as a purine or pyrimidine or a derivative thereof. A nucleobase may be a canonical base (e.g., adenine, guanine, uracil, and cytosine) or a non-canonical or modified base including one or more substitutions or modifications including but not limited to alkyl, aryl, halo, oxo, hydroxyl, alkyloxy, and/or thio substitutions; one or more fused or open rings; oxidation; and/or reduction. Thus, a nucleobase may be selected from the non-limiting group consisting of adenine, guanine, uracil, cytosine, 7-methylguanine, 5-methylcytosine, 5-hydroxymethylcytosine, thymine, pseudouracil, dihydrouracil, hypoxanthine, and xanthine. A nucleoside of an mRNA is a compound including a sugar molecule (e.g., a 5-carbon or 6-carbon sugar, such as pentose, ribose, arabinose, xylose, glucose, galactose, or a deoxy derivative thereof) in combination with a nucleobase. A nucleoside may be a canonical nucleoside (e.g., adenosine, guanosine, cytidine, uridine, 5-methyluridine, deoxyadenosine, deoxyguanosine, deoxycytidine, deoxyuridine, and thymidine) or an analogue thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase and/or sugar component. A nucleotide of an mRNA is a compound containing a nucleoside and a phosphate group or alternative group (e.g., boranophosphate, thiophosphate, selenophosphate, phosphonate, alkyl group, amidate, and glycerol). A nucleotide may be a canonical nucleotide (e.g., adenosine, guanosine, cytidine, uridine, 5-methyluridine, deoxyadenosine, deoxyguanosine, deoxycytidine, deoxyuridine, and thymidine monophosphates) or an analogue thereof and may include one or more substitutions or modifications including but not limited to alkyl, aryl, halo, oxo, hydroxyl, alkyloxy, and/or thio substitutions; one or more fused or open rings; oxidation; and/or reduction of the nucleobase, sugar, and/or phosphate or alternative component. A nucleotide may include one or more phosphate or alternative groups. For example, a nucleotide may include a nucleoside and a triphosphate group. A “nucleoside triphosphate” (e.g., guanosine triphosphate, adenosine triphosphate, cytidine triphosphate, and uridine triphosphate) may refer to the canonical nucleoside triphosphate or an analogue or derivative thereof and may include one or more substitutions or modifications as described herein. For example, “guanosine triphosphate” should be understood to include the canonical guanosine triphosphate, 7-methylguanosine triphosphate, or any other definition encompassed herein. An mRNA may include a 5′ untranslated region, a 3′ untranslated region, and/or a coding or translating sequence. An mRNA may include any number of base pairs, including tens, hundreds, or thousands of base pairs. Any number (e.g., all, some, or none) of nucleobases, nucleosides, or nucleotides may be an analogue of a canonical species, substituted, modified, or otherwise non-naturally occurring. In certain embodiments, all of a particular nucleobase type may be modified. For example, all cytosines in an mRNA may be 5-methylcytosine. In some embodiments, an mRNA may include a 5′ cap structure, a chain terminating nucleotide, a stem loop, a polyA sequence, and/or a polyadenylation signal. A cap structure or cap species is a compound including two nucleoside moieties joined by a linker which caps the mRNA at its 5′ end, and which may be selected from a naturally occurring cap, a non-naturally occurring cap or cap analogue, or an anti-reverse cap analogue (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m7G(5′)ppp(5′)G, commonly written as m7GpppG. A cap species may also be an anti-reverse cap analogue. A non-limiting list of possible cap species includes m7GpppG, m7Gpppm7G, m73′dGpppG, iri27′03′GpppG, iri27′03′GppppG, iri27′02′GppppG, m7Gpppm7G, m73′dGpppG, iri27′03′GpppG, iri27′03′GppppG, and m27 02′GppppG. An mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. An mRNA may instead or additionally include a stem loop, such as a histone stem loop. A stem loop may include 1, 2, 3, 4, 5, 6, 7, 8, 9 or more nucleotide base pairs. For example, a stem loop may include 4, 5, 6, 7, 8, 9 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. An mRNA may instead or additionally include a polyA sequence and/or polyadenylation signal. A polyA sequence may be comprised entirely or mostly of adenine nucleotides or analogues or derivatives thereof. A polyA sequence may be a tail located adjacent to a 3′ untranslated region of an mRNA. The modified mRNA of the present invention may comprise, in addition to the coding region (which may be codon-optimized), one or more of a stem loop, a chain terminating nucleoside, miRNA binding sites, a polyA sequence, a polyadenylation signal, 3′ and/or 5′ untranslated regions (3′ UTRs and/or 5′ UTRs) and/or a 5′ cap structure. Various nucleotide modifications are preferably incorporated into the mRNA to modify it for increased translation, reduced immunogenicity, and increased stability. Suitable modified nucleotides include but are not limited to N1-methyladenosine (m1A), N6-methyladenosine (m6A), 5-methylcytidine (m5C), 5-methyluridine (m5U), 2-thiouridine (s2U), 5-methoxyuridine (5moU), pseudouridine (ψ), N1-methylpseudouridine (m1ψ). Among these mRNA modifications, m5C and ψ are the most preferred as they reduce the immunogenicity of mRNA as well as increase the translation efficiency in vivo. In preferred embodiments of the present invention, the compositions described herein comprise a non-viral delivery vector such as an LNP or a liposome comprising a modified mRNA as the polynucleotide cargo, wherein the mRNA is modified with at least 50% m5C and 50% ψ or m1ψ, preferably at least 75% m5C and 75% ψ or m1ψ, and even more preferably 90% m5C and 90% ψ or m1ψ, or even more preferably 100% modification using m5C and ψ or m1ψ.

A “nucleic acid” refers to a polynucleotide and includes polyribonucleotides and poly-deoxyribonucleotides. Nucleic acids according to the present invention may include any polymer or oligomer of pyrimidine and purine bases, e.g., cytosine, thymine, and uracil, and adenine and guanine, respectively. (See Albert L. Lehninger, Principles of Biochemistry, at 793-800 (Worth Pub. 1982) and G. Michael Blackburn, Michael J. Gait, David Loakes and David M. Williams, Nucleic Acids in Chemistry and Biology 3^{rd} edition, (RSC publishing 2006) which are herein incorporated in their entirety for all purposes). Indeed, the present invention contemplates any deoxyribonucleotide or ribonucleotide component, and any chemical variants thereof. The polymers or oligomers may be heterogeneous or homogeneous in composition, and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, at least 8, at least 15 or at least 25 nucleotides in length, but may be up to 50, 100, 1000, 5000, 10000, 15000, or 20000 nucleotides long or a compound that specifically hybridises to a polynucleotide. Polynucleotides include sequences of DNA or RNA or mimetics thereof, which may be isolated from natural sources, recombinantly produced or artificially synthesised. A further example of a polynucleotide as employed in the present invention may be a peptide nucleic acid (PNA; see U.S. Pat. No. 6,156,501, which is hereby incorporated by reference in its entirety.) The invention also encompasses situations in which there is a non-traditional base pairing, such as Hoogsteen base pairing, which has been identified in certain tRNA molecules and postulated to exist in a triple helix. “Polynucleotide” and “oligonucleotide” are used interchangeably herein. It will be understood that when a nucleotide sequence is represented herein by a DNA sequence (e.g., A, T, G, and C), this also includes the corresponding RNA sequence (e.g., A, U, G, C) in which “U” replaces “T”.

As used herein, “polynucleotide” includes, for instance, cDNA, RNA, DNA/RNA hybrid, antisense RNA, siRNA, mRNA, ribozyme, genomic DNA, synthetic forms, and mixed polymers, both sense and antisense strands, and may be chemically or biochemically modified to contain non-natural or derivatised, synthetic, or semi-synthetic nucleotide bases. Also, contemplated are alterations of a wild type or synthetic gene, including but not limited to deletion, insertion, substitution of one or more nucleotides, or fusion to other polynucleotide sequences, such as mRNA molecules, plasmids or mini-circles.

Non-limiting examples of proteins of interest (PoIs) that may be delivered by the peptides of the present invention or encoded by the polynucleotide cargos of the invention (such as mRNAs, plasmids and mini-circles) include: antibodies, intrabodies, nanobodies, single chain variable fragments (scFv), affibodies, bi- and multispecific antibodies or binders, receptors, ligands, transporters, enzymes for e.g. ERT or gene editing, tumour suppressors, viral or bacterial inhibitors, cell component proteins, DNA and/or RNA binding proteins, DNA repair inhibitors, nucleases, proteinases, integrases, transcription factors, growth factors, apoptosis inhibitors and inducers, toxins (for instance Pseudomonas exotoxins), structural proteins, neurotrophic factors such as NT3/4, brain-derived neurotrophic factor (BDNF) and nerve growth factor (NGF) and its individual subunits such as the 2.5S beta subunit, ion channels, membrane transporters, proteostasis factors, proteins involved in cellular signaling, translation- and transcription related proteins, nucleotide binding proteins, protein binding proteins, lipid binding proteins, glycosaminoglycans (GAGs) and GAG-binding proteins, metabolic proteins, cellular stress regulating proteins, inflammation and immune system regulating proteins, mitochondrial proteins, and heat shock proteins, etc. In one preferred embodiment, the encoded protein is a CRISPR-associated (Cas) polypeptide (such as Cas9) with intact nuclease activity which is associated with (i.e. carries with it) an RNA strand that enables the Cas polypeptide to carry out its nuclease activity in a target cell once delivered by the peptide. Alternatively, in another preferred embodiment, the Cas polypeptide may be catalytically inactive, to enable targeted genetic engineering. Yet another alternative may be any other type of CRISPR effector such as the single RNA guided endonuclease Cpf1. The inclusion of Cpf1 is a particularly preferred embodiment of the present invention, as it cleaves target DNA via a staggered double-stranded break. Cpf1 may be obtained from species such as Acidaminococcus or Lachnospiraceae. In yet another exemplary embodiment, the Cas polypeptide may also be fused to a transcriptional activator (such as the P3330 core protein), to specifically induce gene expression. Additional preferred embodiments include proteins selected from the group comprising enzymes or transporters for lysosomal storage disorders, for instance glucocerebrosidases such as imiglucerase, alpha-galactosidase, alpha-L-iduronidase, iduronate-2-sulfatase and idursulfase, arylsulfatase, galsulfase, acid-alpha glucosidase, sphingomyelinase, galactocerebrosidase, galactosylceramidase, ceramidase, alpha-N-acetylgalactosaminidase, beta-galactosidase, lysosomal acid lipase, acid sphingomyelinase, NPC1, NPC2, heparan sulfamidase, N-acetylglucosaminidase, heparan-α-glucosaminide-N-acetyltransferase, N-acetylglucosamine 6-sulfatase, galactose-6-sulfate sulfatase, galactose-6-sulfate sulfatase, hyaluronidase, alphaN-acetyl neuraminidase, GlcNAc phosphotransferase, mucolipin1, palmitoylprotein thioesterase, tripeptidyl peptidase I, palmitoyl-protein thioesterase 1, tripeptidyl peptidase 1, battenin, linclin, alpha-D-mannosidase, beta-mannosidase, aspartylglucosaminidase, alpha-L-fucosidase, cystinosin, cathepsin K, sialin, LAMP2, and hexoaminidase. In other preferred embodiments, the Pol may be e.g. an intracellular protein that modifies inflammatory responses, for instance epigenetic proteins such as methylases and bromodomains, or an intracellular protein that modifies muscle function, e.g. transcription factors such as MyoD or Myf5, proteins regulating muscle contractility e.g. myosin, actin, calcium/binding proteins such as troponin, or structural proteins such as Dystrophin, mini-dystrophin, micro-dystrophin, utrophin, titin, nebulin, dystrophin-associated proteins such as dystrobrevin, syntrophin, syncoilin, desmin, sarcoglycan, dystroglycan, sarcospan, agrin, and/or fukutin. The PoIs are typically proteins or peptides of human origin unless indicated otherwise by their name, any other nomenclature, or as known to a person skilled in the art, and they can be found in various publicly available databases such as Uniprot, RCSB, etc.

The terms “extracellular vesicle” or “EV” or “exosome” shall be understood to relate to any type of vesicle that is, for instance, obtainable from a cell, for instance a microvesicle (e.g. any vesicle shed from the plasma membrane of a cell, for instance an ARRDC1-mediated microvesicle (a so-called ARMM), an exosome (e.g. any vesicle derived from the endosomal, lysosomal or endo-lysosomal pathway), an apoptotic body (e.g. obtainable from apoptotic cells), a microparticle (which may be derived from e.g. platelets), an ectosome (derivable from e.g. neutrophils and monocytes in serum), prostatosome (e.g. obtainable from prostate cancer cells), or a cardiosome (e.g. derivable from cardiac cells) etc. Furthermore, the said terms shall also be understood to relate to lipoprotein particles, such as low-density lipoprotein (LDL), very low-density lipoprotein (VLDL), high-density lipoprotein (HDL) and chylomicrons, as well as EV mimics, cellular membrane vesicles obtained through membrane extrusion or other techniques, etc. Essentially, the EV may be any type of lipid-based structure (with vesicular morphology or with any other type of suitable morphology) that can act as a delivery or transport vehicle.

The terms “source cell” or “EV source cell” or “parental cell” or “cell source” or “EV-producing cell” or any other similar terminology shall be understood to relate to any type of cell that is capable of producing EVs under suitable conditions, typically in cell culture. Cell culture may include suspension culture, adherent culture or any other type of culturing system, in vivo, ex vivo and/or in vitro and may be generated as stable cell lines or single clones. Source cells for use with the present invention may also include cells producing exosomes in vivo, e.g. via delivery of a polynucleotide construct into a subject for subsequent translation and in vivo production of EVs, in e.g. the liver. The most advantageous source cells for use with the present invention are mesenchymal stem cells (MSCs), amnion-derived cells, amnion epithelial (AE) cells, human embryonic kidney (HEK) cells, such as HEK293, HEK293T, or suspension HEK cells, such as the Freestyle HEK293-F cells, endothelial cells, epithelial cells, lymphocytes, leukocytes, red blood cells (erythrocytes), hematopoietic cells and progenitor cells, any perinatal cells, and/or placenta-derived cells, all of which are of mammal, most preferably of human, origin. The MSCs may be obtained from e.g. bone marrow, adipose tissue, perinatal tissue (e.g. amnion, amniotic membrane, amniotic fluid, chorion, placenta, umbilical cord, Wharton's jelly, etc.), tooth buds, umbilical cord blood, skin tissue, etc. Generally, EVs may be derived from essentially any cell source, be it a primary cell source or an immortalised cell line. The EV source cells may be any embryonic, fetal or adult somatic stem cell types, including induced pluripotent stem cells (iPSCs) and other stem cells derived by any method, as well as any adult cell source. The source cell may be either allogeneic, autologous or even xenogeneic in nature to the patient to be treated, i.e. the cells may be from the patient himself or from an unrelated or related, matched or unmatched donor. In certain contexts, allogeneic cells may be preferable from a medical standpoint, as they could provide immuno-modulatory effects that may not be obtainable from autologous cells of a patient suffering from a certain indication.

The terms “patient,” “subject,” “individual,” and the like are used interchangeably herein, and refer to any animal, or cells thereof whether in vitro or in situ, amenable to the methods and treatments described herein. In certain non-limiting embodiments, the patient, subject or individual is a human.

In a further embodiment, the EVs carried as cargo by the peptides of the present invention may themselves be loaded with therapeutic cargo molecules such as: mRNA, antisense or splice-switching oligonucleotides, siRNA, shRNA, miRNA, pDNA, supercoiled or unsupercoiled plasmids, mini-circles, peptides, proteins, antibodies, antibody-drug conjugates, small molecule drugs, gene editing technology such as CRISPR-Cas9, TALENs, meganucleases, or vesicle-based cargos such as viruses (e.g. AAVs, lentiviruses, etc.). These cargos may be loaded by endogenous or exogenous loading mechanisms known from the literature.

Alternatively, or in addition, the EVs carried as cargo by the peptides of the present invention may comprise at least one targeting moiety, to enable targeted delivery to a cell, tissue, organ, and/or compartment of interest. The targeting moiety may be comprised in a fusion polypeptide, which is especially advantageous when using an exosomal polypeptide with a transmembrane domain to enable display of the targeting moiety on the surface of the EVs. Targeting moieties may be short peptides of 5 to 50, 5 to 70, 5 to 100, 5 to 150, 5 to 170, 5 to 200, 5 to 250 amino acids in length, proteins, receptors, ligands, peptides, single chain fragments, single domain antibodies, nanobodies, or any other derivatives of antibodies, such as a VHH, a VNAR, an alphabody, an affibody, a centyrin, heavy chain only antibodies, a humabody, or a nanobody. Targeting moieties may be used to target the EVs to cells, subcellular locations, tissues, organs or other bodily compartments. Organs and cell types that may be targeted include: the brain, neuronal cells, the blood brain barrier, muscle tissue, the eye, lungs, liver, kidneys, heart, stomach, intestines, pancreas, red blood cells, white blood cells including B cells and T cells, lymph nodes, bone marrow, spleen and cancer cells. Targeting can be achieved by a variety of means, for instance the use of targeting peptides. Such targeting peptides may be anywhere from a few amino acids in length to several 100s of amino acids in length, e.g. anywhere in the interval of 3-200 amino acids, 3-100 amino acids, 5-30 amino acids, 5-25 amino acids, e.g. 7 amino acids, 12 amino acids, 20 amino acids, etc. Targeting peptides of the present invention may also include full length proteins such as receptors, receptor ligands, etc. Exemplary targeting moieties include brain targeting moieties such as rabies virus glycoprotein (RVG), NGF, melanotransferrin and the scFv FC5. Peptide and muscle targeting include moieties such as Muscle Specific Peptide (MSP).

The targeting moiety may also form part of a separate polypeptide construct which is comprised in the EV. Further, the fusion polypeptides comprised in the EVs may also comprise various additional moieties to enhance bioactive delivery. Such moieties and/or domains may include the following non-limiting examples of functional domains: (i) multimerisation domains which dimerise, trimerise, or multimerise the fusion polypeptides to improve EV formation and/or loading, (ii) linkers, as above-mentioned, to avoid steric hindrance and provide flexibility, (iii) release domains, such as cis-cleaving elements like inteins, which have self-cleaving activity which is useful for release of particular parts of the fusion polypeptide and/or the nucleic acid cargo, (iv) RNA cleaving domains for improved release of the nucleic acid in recipient cells, for instance domains encoding nucleases such as Cas6, Cas13, (v) endosomal escape domains, such as HA2, VSVG, GALA, B18, etc., and/or (vi) nuclear localisation signals (NLSs). When the cargo is a targeted EV, the advantage is clearly that the peptide assists the delivery whilst the targeting moiety on the EV enables that delivery to be specifically tailored to the organ or cell type of interest.

When the targeting moiety on the EV is formed as part of a fusion protein with an exosomal polypeptide, the exosomal polypeptide may be selected from the group consisting of the following non-limiting examples: CD9, CD53, CD63, CD81, CD54, CD50, FLOT1, FLOT2, CD49d, CD71, CD133, CD138, CD235a, ALIX, Syntenin-1, Syntenin-2, Lamp2b, TSPAN8, syndecan-1, syndecan-2, syndecan-3, syndecan-4, TSPAN14, CD37, CD82, CD151, CD231, CD102, NOTCH1, NOTCH2, NOTCH3, NOTCH4, DLL1, DLL4, JAG1, JAG2, CD49d/ITGA4, ITGB5, ITGB6, ITGB7, CD11a, CD11 b, CD11c, CD18/ITGB2, CD41, CD49b, CD49c, CD49e, CD51, CD61, CD104, CD2, CD3 epsilon, CD3 zeta, CD13, CD18, CD19, CD30, CD34, CD36, CD40, CD40L, CD44, CD45, CD45RA, CD47, CD86, CD110, CD111, CD115, CD117, CD125, CD135, CD184, CD200, CD279, CD273, CD 274, CD362, COL6A1, AGRN, EGFR, GAPDH, GLUR2, GLUR3, HLA-DM, HSPG2, L1 CAM, LAMB1, LAMC1, ARRDC1, PDGFRN, ATP2B2, ATP2B3, ATP2B4, BSG, IGSF2, IGSF3, IGSF8, ITGB1, ATP1A2, ATP1A3, ATP1A4, ITGA4, SLC3A2, ATP1A1, ATP1B3, ATP2B1, LFA-1, LGALS3BP, Mac-1 alpha, Mac-1 beta, MFGE8, SLIT2, STX3, TCRA, TCRB, TCRD, TCRG, VTI1A, VTI1B, and any other EV proteins, and any combinations, derivatives, domains, variants, mutants, or regions thereof. Particularly advantageous EV proteins include CD63, CD81, CD9, CD82, CD44, CD47, CD55, LAMP2B, ICAMs, integrins, ARRDC1, syndecan, syntenin, and Alix, as well as derivatives, domains, variants, mutants, or regions thereof.

As used herein, “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system.

Both the coding strand, the nucleotide sequence of which is identical to the mRNA sequence, and the non-coding strand, used as the template for transcription of a gene or cDNA, can be referred to as encoding the protein or other product of that gene or cDNA.

The present invention also relates to a pharmaceutical composition comprising a peptide of the present invention and a pharmaceutically acceptable carrier. The peptide may be attached to a cargo of therapeutic nucleic acid, therapeutic protein or therapeutic vesicle as described herein. The composition may additionally comprise a pharmaceutically acceptable excipient and/or diluent or similar.

The pharmaceutically acceptable carrier may comprise an aqueous solution, for example an aqueous solution comprising an additive, such as a buffering agent (e.g. HEPES, sodium acetate, sodium succinate, sodium citrate, potassium phosphate based buffers, or similar). The pharmaceutically acceptable carrier may optionally also comprise one or more other additives, including mono-, di- and polysaccharides (such as glucose, fructose, galactose, sucrose, lactose, trehalose or others), sugar alcohols (such as sorbitol, mannitol, glycerol), dihydric alcohol, polymers (such as polyethylene glycols, propylene glycol, polyvinyl alcohols (PVA; such as PVA18, PVA40 or PVA-PEG), polyvinylpyrrolidone (PVP; such as PVP10 or PVP55) or surfactants (such as sorbitan esters, including Tween 20, Tween 40, Tween 65, Tween 80, or polyoxyethylene-polyoxypropylene block copolymers, such as Pluronic F68 and Pluronic F127, or others).

Furthermore, the present invention also pertains to the peptides of the present invention attached to the nucleic acid, protein or vesicle cargo as described herein, for use in medicine. As abovementioned, the nucleic acid, protein, vesicle or any other drug cargo may be attached to the peptide covalently or non-covalently, e.g. via electrostatic interactions, van der Waals interactions, hydrogen bonding, or general ionic interactions. Covalent attachment to the peptide has the advantage that it gives a more defined product in the form of a conjugate between the peptide and drug cargo, which is linked via a covalent bond, for instance a peptide bond or an amide bond, an ester bond, a thioester bond, a disulphide bridge, etc. Non-covalent interactions, on the other hand, advantageously result in the formation of nanoparticles which are efficiently internalised into target cells in vitro, ex vivo and in vivo. The size of said nanoparticles varies according to the drug cargo in question, with complexation of mRNA typically resulting in nanoparticles with a mean diameter in the range between 100-400 nm, more preferably 200-300 nm. However, the size of the peptide/mRNA nanoparticles may vary depending on the molar ratio between the peptide and mRNA, with a higher molar ratio typically resulting in larger particles. At the most efficient molar ratios, the peptide/mRNA nanoparticles of the present invention typically exhibit a positive zeta potential, but the zeta potential may at certain molar ratios also be negative and/or neutral (i.e. zero or around zero). The size of the nanoparticles and the zeta potential may also be influenced by the buffer in which the nanoparticles are present, as a result of ionic strength, pH or the presence and/or concentration of various salts. Analogously, nanoparticles formed between the peptides of the present invention and various short nucleic acid molecules, such as antisense oligonucleotides or siRNAs, typically have a mean diameter in the range between 50-400 nm, normally 75-200 nm, with a zeta potential that is typically positive but which may be negative and/or close to neutral or neutral (zero). Again, buffer selection is important for the formation, stability and size of the nanoparticles regardless of therapeutic cargo molecule.

Further, the size and zeta potential of the nanoparticles formed between the peptides of the present invention and RNPs or vesicles (such as EVs, exosomes, microvesicles, viruses, etc.) may vary according to the peptide in question, the molar ratio, the size of the RNP and/or the vesicle-based cargo (e.g. an AAV or an exosome), but the nanoparticles are typically in the nanometer range, i.e. below 1 μm, preferably 50-500 nm.

Thus, the present invention provides any of the peptides as described herein for use in therapy. In an embodiment, the peptide is bound to any of the cargo molecules as described herein. A method of medical treatment comprising administering a peptide of the invention, optionally bound to a cargo molecule as described herein, to a subject in need thereof is also provided.

Importantly, the present invention relates to use of the peptides described herein in the prophylaxis and/or treatment and/or alleviation of a variety of diseases, typically via the delivery of essentially any type of drug cargo, such as, for instance, mRNA, antisense or splice-switching oligonucleotides, siRNA, shRNA, miRNA, pDNA, peptides, proteins, antibodies, antibody-drug conjugates, small molecule drugs, gene editing technology such as CRISPR-Cas9, TALENs, meganucleases, or vesicle-based cargos such as viruses (e.g. AAVs, lentiviruses, etc.) or EVs (exosomes, microvesicles and the like, specifically those loaded with therapeutic cargo molecules).

As used herein a “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

The terms “therapy” or “therapeutic regimen” refer to those activities taken to alleviate or alter a disorder or disease state, e.g., a course of treatment intended to reduce or eliminate at least one sign or symptom of a disease or disorder using pharmacological, surgical, dietary and/or other techniques. A therapeutic regimen may include a prescribed dosage of one or more drugs or surgery. Therapies will most often be beneficial and reduce or eliminate at least one sign or symptom of the disorder or disease state, but in some instances the effect of a therapy will have non-desirable or side-effects. The effect of therapy will also be impacted by the physiological state of the subject, e.g., age, gender, genetics, weight, other disease conditions, etc.

To “treat” a disease as the term is used herein, means to reduce the frequency or severity of at least one sign or symptom of a disease or disorder experienced by a subject.

Non-limiting examples of diseases and conditions that are suitable targets for treatment using the peptide delivery system described herein include the following non-limiting examples: Crohn's disease, ulcerative colitis, ankylosing spondylitis, rheumatoid arthritis, multiple sclerosis, systemic lupus erythematosus, sarcoidosis, idiopathic pulmonary fibrosis, psoriasis, tumor necrosis factor (TN F) receptor-associated periodic syndrome (TRAPS), deficiency of the interleukin-1 receptor antagonist (DIRA), endometriosis, autoimmune hepatitis, scleroderma, myositis, stroke, acute spinal cord injury, vasculitis, Guillain-Barré syndrome, acute myocardial infarction, ARDS, sepsis, meningitis, encephalitis, liver failure, non-alcoholic steatohepatitis (NASH), non-alcoholic fatty liver disease (NAFLD), kidney failure, heart failure or any acute or chronic organ failure and the associated underlying etiology, graft-vs-host disease, Duchenne muscular dystrophy and other muscular dystrophies, In-born errors of metabolism including: Disorders of carbohydrate metabolism e.g., G6PD deficiency galactosemia, hereditary fructose intolerance, fructose 1,6-diphosphatase deficiency and the glycogen storage diseases, Disorders of organic acid metabolism (organic acidurias) such as alkaptonuria, 2-hydroxyglutaric acidurias, methylmalonic or propionic acidemia, multiple carboxylase deficiency′ Disorders of amino acid metabolism such as phenylketonuria, maple syrup urine disease, glutaric acidemia type 1, Aminoacidopathies e.g., hereditary tyrosinemia, nonketotic hyperglycinemia, and homocystinuria, Hereditary tyrosinemia, Fanconi syndrome, Primary Lactic Acidoses e.g., pyruvate dehydrogenase, pyruvate carboxylase and cytochrome oxidase deficiencies, Disorders of fatty acid oxidation and mitochondrial metabolism such as short, medium, and long-chain acyl-CoA dehydrogenase deficiencies also known as Beta-oxidation defects, Reye's syndrome, Medium-chain acyl-coenzyme A dehydrogenase deficiency (MCADD), mitochondrial encephalopathy lactic acidosis and stroke-like episodes (MELAS), myoclonic epilepsy with ragged red fibers (MERFF), pyruvate dehydrogenase deficiency, Disorders of porphyrin metabolism such as acute intermittent porphyria, Disorders of purine or pyrimidine metabolism such as Lesch-Nyhan syndrome′ Disorders of steroid metabolism such as lipoid congenital adrenal hyperplasia, congenital adrenal hyperplasia, Disorders of mitochondrial function such as Kearns-Sayre syndrome, Disorders of peroxisomal function such as Zellweger syndrome and neonatal adrenoleukodystrophy, congenital adrenal hyperplasia or SmithLemli-Opitz, Menkes syndrome, neonatal hemochromatosis, Urea cycle disorders such as N-Acetylglutamate synthase deficiency, carbamoyl phosphate synthetase deficiency, ornithine transcarbamoylase deficiency, citrullinemia (deficiency of argininosuccinic acid synthase), argininosuccinic aciduria (deficiency of argininosuccinic acid lyase), argininemia (deficiency of arginase), hyperornithinemia, hyperammonemia, homocitrullinuria (HHH) syndrome (deficiency of the mitochondrial ornithine transporter), citrullinemia II (deficiency of citrin, an aspartate glutamate transporter), lysinuric protein intolerance (mutation in y+L amino acid transporter 1, orotic aciduria (deficiency in the enzyme uridine monophosphate synthase UMPS), all of the lysosomal storage diseases, for instance Alpha-mannosidosis, Betamannosidosis, Aspartylglucosaminuria, Cholesteryl Ester Storage Disease, Cystinosis, Danon Disease, Fabry Disease, Farber Disease, Fucosidosis, Galactosialidosis, Gaucher Disease Type I, Gaucher Disease Type II, Gaucher Disease Type III, GM1 Gangliosidosis Type I, GM1 Gangliosidosis Type II, GM1 Gangliosidosis Type III, GM2—Sandhoff disease, GM2—Tay-Sachs disease, GM2—Gangliosidosis, AB variant, Mucolipidosis II, Krabbe Disease, Lysosomal acid lipase deficiency, Metachromatic Leukodystrophy, MPS I—Hurler Syndrome, MPS I—Scheie Syndrome, MPS I Hurler-Scheie Syndrome, MPS II—Hunter Syndrome, MPS IIIA—Sanfilippo Syndrome Type A, MPS IIIB—Sanfilippo Syndrome Type B, MPS IIIB—Sanfilippo Syndrome Type C, MPS IIIB—Sanfilippo Syndrome Type D, MPS IV Morquio Type A, MPS IV—Morquio Type B, MPS IX—Hyaluronidase Deficiency, MPS VI—Maroteaux-Lamy, MPS VII—Sly Syndrome, Mucolipidosis I—Sialidosis, Mucolipidosis IIIC, Mucolipidosis Type IV, Mucopolysaccharidosis, Multiple Sulfatase Deficiency, Neuronal Ceroid Lipofuscinosis T1, Neuronal Ceroid Lipofuscinosis T2, Neuronal Ceroid Lipofuscinosis T3, Neuronal Ceroid Lipofuscinosis T4, Neuronal Ceroid Lipofuscinosis T5, Neuronal Ceroid Lipofuscinosis T6, Neuronal Ceroid Lipofuscinosis T7, Neuronal Ceroid Lipofuscinosis T8, Neuronal Ceroid Lipofuscinosis T9, Neuronal Ceroid Lipofuscinosis T10, Niemann-Pick Disease Type A, Niemann-Pick Disease Type B, Niemann-Pick Disease Type C, Pompe Disease, Pycnodysostosis, Salla Disease, Schindler Disease and Wolman Disease, etc. cystic fibrosis, primary ciliary dyskinesia, pulmonary alveolar proteinosis, ARC syndrome, Ret syndrome, neurodegenerative diseases including Alzheimer's disease, Parkinson's disease, GBA associated Parkinson's disease, Huntington's disease and other trinucleotide repeat-related diseases, dementia, ALS, cancer-induced cachexia, anorexia, diabetes mellitus type 2, and various cancers. Virtually all types of cancer are relevant disease targets for the present invention, for instance, Acute lymphoblastic leukemia (ALL), Acute myeloid leukemia, Adrenocortical carcinoma, AIDS-related cancers, AIDS-related lymphoma, Anal cancer, Appendix cancer, Astrocytoma, cerebellar or cerebral, Basal-cell carcinoma, Bile duct cancer, Bladder cancer, Bone tumor, Brainstem glioma, Brain cancer, Brain tumor (cerebellar astrocytoma, cerebral astrocytoma/malignant glioma, ependymoma, medulloblastoma, supratentorial primitive neuroectodermal tumors, visual pathway and hypothalamic glioma), Breast cancer, Bronchial adenomas/carcinoids, Burkitt's lymphoma, Carcinoid tumor (childhood, gastrointestinal), Carcinoma of unknown primary, Central nervous system lymphoma, Cerebellar astrocytoma/Malignant glioma, Cervical cancer, Chronic lymphocytic leukemia, Chronic myelogenous leukemia, Chronic myeloproliferative disorders, Colon Cancer, Cutaneous T-cell lymphoma, Desmoplastic small round cell tumor, Endometrial cancer, Ependymoma, Esophageal cancer, Extracranial germ cell tumor, Extragonadal Germ cell tumor, Extrahepatic bile duct cancer, Eye Cancer (Intraocular melanoma, Retinoblastoma), Gallbladder cancer, Gastric (Stomach) cancer, Gastrointestinal Carcinoid Tumor, Gastrointestinal stromal tumor (GIST), Germ cell tumor (extracranial, extragonadal, or ovarian), Gestational trophoblastic tumor, Glioma (glioma of the brain stem, Cerebral Astrocytoma, Visual Pathway and Hypothalamic glioma), Gastric carcinoid, Hairy cell leukemia, Head and neck cancer, Heart cancer, Hepatocellular (liver) cancer, Hodgkin lymphoma, Hypopharyngeal cancer, Intraocular Melanoma, Islet Cell Carcinoma (Endocrine Pancreas), Kaposi sarcoma, Kidney cancer (renal cell cancer), Laryngeal Cancer, Leukemias ((acute lymphoblastic (also called acute lymphocytic leukemia), acute myeloid (also called acute myelogenous leukemia), chronic lymphocytic (also called chronic lymphocytic leukemia), chronic myelogenous (also called chronic myeloid leukemia), hairy cell leukemia)), Lip and Oral Cavity Cancer, Liposarcoma, Liver Cancer (Primary), Lung Cancer (Non-Small Cell, Small Cell), Lymphomas, AIDS-related lymphoma, Burkitt lymphoma, cutaneous T-Cell lymphoma, Hodgkin lymphoma, Non-Hodgkin, Medulloblastoma, Merkel Cell Carcinoma, Mesothelioma, Metastatic Squamous Neck Cancer with Occult Primary, Mouth Cancer, Multiple Endocrine Neoplasia Syndrome, Multiple Myeloma/Plasma Cell Neoplasm, Mycosis Fungoides, Myelodysplastic/Myeloproliferative Diseases, Myelogenous Leukemia, Chronic Myeloid Leukemia (Acute, Chronic), Myeloma, Nasal cavity and paranasal sinus cancer, Nasopharyngeal carcinoma, Neuroblastoma, Oral Cancer, Oropharyngeal cancer, Osteosarcoma/malignant fibrous histiocytoma of bone, Ovarian cancer, Ovarian epithelial cancer (Surface epithelial-stromal tumor), Ovarian germ cell tumor, Ovarian low malignant potential tumor, Pancreatic cancer, Pancreatic islet cell cancer, Parathyroid cancer, Penile cancer, Pharyngeal cancer, Pheochromocytoma, Pineal astrocytoma, Pineal germinoma, Pineoblastoma and supratentorial primitive neuroectodermal tumors, Pituitary adenoma, Pleuropulmonary blastoma, Prostate cancer, Rectal cancer, Renal cell carcinoma (kidney cancer), Retinoblastoma, Rhabdomyosarcoma, Salivary gland cancer, Sarcoma (Ewing family of tumors sarcoma, Kaposi sarcoma, soft tissue sarcoma, uterine sarcoma), Sézary syndrome, Skin cancer (nonmelanoma, melanoma), Small intestine cancer, Squamous cell, Squamous neck cancer, Stomach cancer, Supratentorial primitive neuroectodermal tumor, Testicular cancer, Throat cancer, Thymoma and Thymic carcinoma, Thyroid cancer, Transitional cell cancer of the renal pelvis and ureter, Urethral cancer, Uterine cancer, Uterine sarcoma, Vaginal cancer, Vulvar cancer, Waldenström macroglobulinemia, and/or Wilm's tumor. The compositions and methods of the present invention are particular useful in the treatment of genetic diseases, lysosomal storage disorders, inborn errors of metabolism, urea cycle disorders, neuromuscular diseases, neurodegenerative diseases, cancer, infectious diseases, autoimmune diseases, kidney diseases, liver diseases, cardiovascular diseases, and inflammatory diseases.

Thus, the present invention encompasses any of the peptides as described herein, optionally bound to any of the cargo molecules as described herein, for use in a method of treating any of the disorders and conditions recited above. Methods of treating any of the disorders and conditions recited above comprising administering a peptide of the invention, optionally bound to a cargo molecule as described herein, to a subject in need thereof are also provided.

The peptide and consequently the drug cargo according to the present invention may be administered to a human or animal subject via various different administration routes, for instance auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracerebroventricular, intracerebroventricular (ICV), intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the characteristics of the pharmaceutical compositions to be administered.

A therapeutically effective amount of a peptide of the invention and cargo molecule may be administered to a patient. The term “therapeutically effective amount” refers to the amount of the peptide/cargo that will elicit the biological or medical response of a tissue, system, or subject that is being sought by the researcher, veterinarian, medical doctor or other clinician. The term “therapeutically effective amount” includes that amount of a peptide/cargo that, when administered, is sufficient to prevent development of, or alleviate to some extent, one or more of the signs or symptoms of the disorder or disease being treated. As such, it means an amount which provides a therapeutic or prophylactic benefit. The therapeutically effective amount will vary depending on the peptide/cargo, the disease and its severity and the age, weight, etc., of the subject to be treated.

The invention and its various aspects, embodiments, alternatives, and variants will now be further exemplified with the enclosed examples, which naturally also may be modified considerably without departing from the scope and the gist of the invention.

### Example 1—Delivery of SSOs

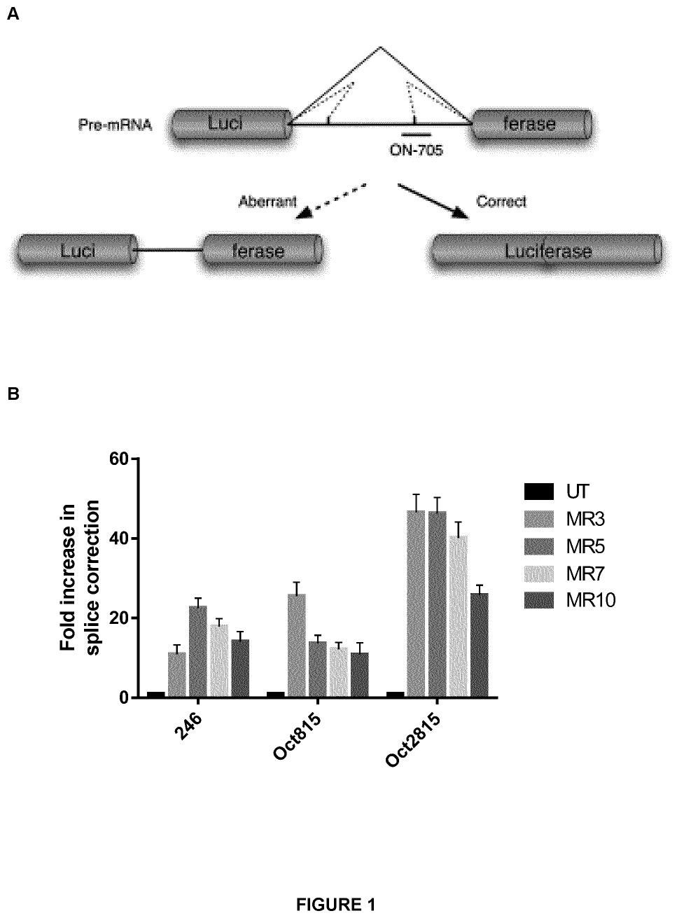

To evaluate the ability of peptides of the invention to effectively deliver SSOs into cells, the HeLa pluc705 splice correction reporter system was used as a model (FIG. 1A). In this assay, HeLa cells are stably transfected with a luciferase endocing sequence that is interrupted by the β-globin pre-mRNA carrying a crypting splice site. If the SSO is effectively delivered to the cells and reaches the cell nucleus, it masks the aberrant splicing site (depicted ON-705 on the right-hand side of FIG. 1A). By redirecting the splicing, it will give rise to the production of functional luciferase protein. The luciferase enzyme activity thus acts as a measure to assess the delivery efficacy of the SSOs. This model is known for being an excellent positive read-out system with wide dynamic range for evaluating and comparing the delivery of SSOs and different delivery vectors.

A range of peptides were tested, including H-L A K L A K A A(R8) A K L L K A A(S5) A K A L-NH2 (SEQ ID NO: 1) (246), H-L A K L A K A A(R8) A K L L K A A(S8) A K A L-NH2 (SEQ ID NO: 3) (Oct815) and H-L A(R8) K L A K A A(R8) A K L L K A A(S8) A K A L-NH2 (SEQ ID NO: 4) (Oct2815), wherein A(R8)=(R)-2-(7-octenyl)alanine, A(S8)=(S)-2-(7-octenyl)alanine and A(S5)=(S)-2-(4-pentenyl)alanine.

The peptides were formulated with 2′-O-methyl phosphorothioate (2′OMe)-based SSOs at different peptide-to-SSO molar ratios (MRs), namely MR3, MR5, MR7 and MR10. The HeLa pluc705 cells were treated with various peptide/SSO nanoformulations at 100 nM SSO concentration for 24 h. After this time, the luciferase activity was measured. Delivery efficacy was expressed as a fold-increase over the values from untreated (UT) cells (ie. fold increase in splice correction).

As shown in FIG. 1B, the tested peptides 246, Oct815 and Oct2815, were all able to mediate effective SSO delivery and induce strong splice switching activity in the HeLa pluc705 cells. The most hydrophobic derivative, Oct2815, demonstrated the greatest potency. Also, with all tested peptides, the highest degree of bioactivity was achieved between MR3 and MR5.

### Example 2—Delivery and Biodistribution of Short Antisense SSOs

In order to investigate the in vivo delivery capacity and biodistribution profile of the peptide/SSO nanoparticles, Oct2815 peptide was formulated at a dose of 50 μg (2.5 mg/kg) of Cy5-labelled 2′-OMe/LNA mixmer-based SSO (at MR10) and then injected intravenously into NMRI mice. After 48 h of treatment, the fluorescence values were measured from the dissected tissues (brain, liver, lungs, heart, G.I. tract, spleen, muscles and kidneys) of the animals with an IVIS in vivo imager.

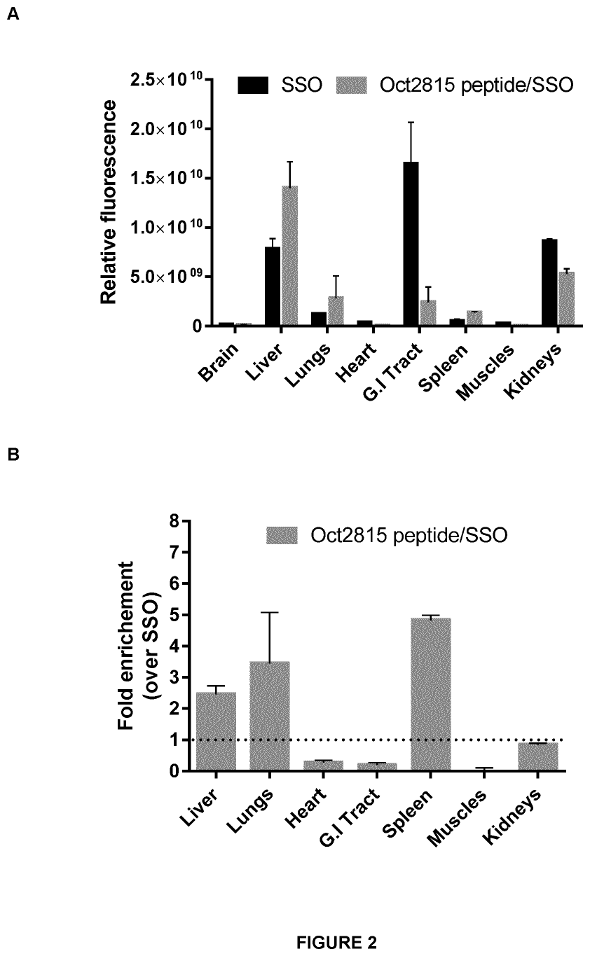

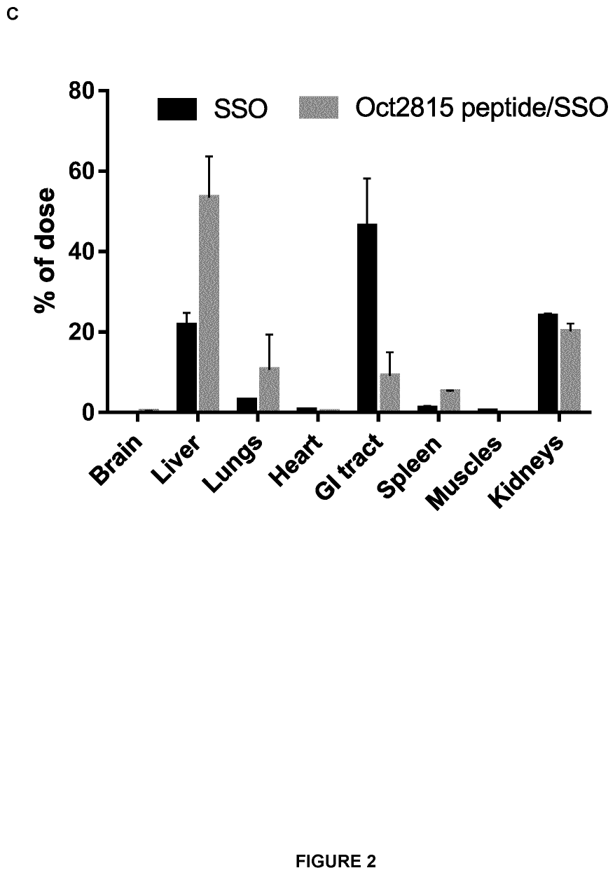

FIG. 2 shows, via three different parameters ((A) relative fluorescence, (B) fold enrichment, and (C) percentage of injected does), that peptide Oct2815/SSO improved the biodistribution of short antisense SSOs in vivo. The results, especially those in FIG. 2(B), show that using Oct2815 to deliver SSO significantly increased the accumulation of the SSOs in liver (2.5-fold), lungs (3.5-fold) and spleen (5-fold) in comparison with SSO alone.

### Example 3—Delivery of Charge Neutral PMO Antisense Oligonucleotides

The PMO used in this experiment was designed to target the alternative spicing of Gp130 (interleukin-6 signal transducer) for inducing the production of soluble Gp130 isoform. The PMO was formulated with Oct2815 peptide and was delivered to HEK293 cells at either 2 uM or 4 uM. The splice switching (exon 9 skipping activity) of this PMO was measured by reverse transcription polymerase chain reaction (RT-PCR) and polyacrylamide gel electrophoresis of the RT-PCR products.

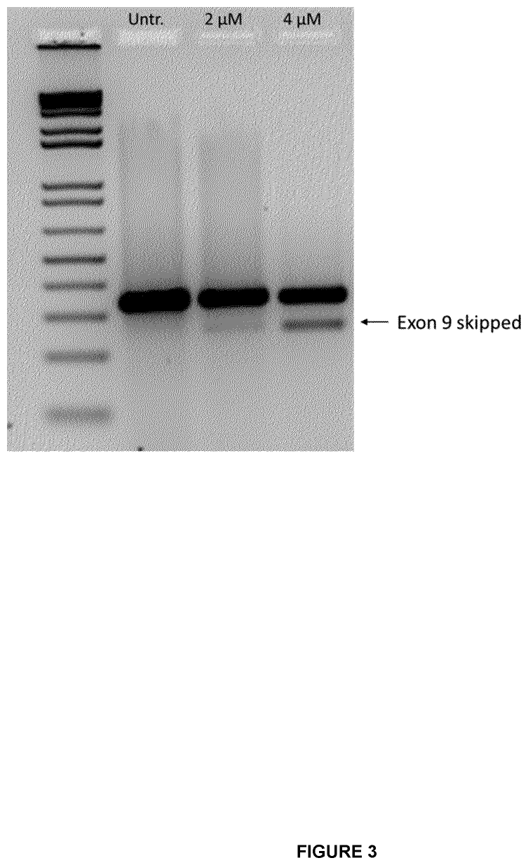

The RT-PCR analysis shown in FIG. 3 demonstrates that the Oct2815 peptide was able to mediate the delivery of charge neutral PMO antisense oligonucleotides to the cells. Moreover, it effected exon skipping to produce an alternative splice variant, which can be seen on the blot.

### Example 4—mRNA Delivery

Delivery of mRNA was tested using the 246, Oct815 and Oct2815 peptides and a luciferase-encoding mRNA (Luc mRNA). Various peptides were formulated at 1.25 mg/kg of mRNA dosing, at a peptide-to-mRNA weight ratio 4. Oct2815 was also formulated at 6.25 mg/kg of mRNA dosing, at a peptide-to-mRNA weight ratio 4. The formulations were subsequently injected intravenously into NMRI mice. After 24 h of treatment, the mice were sacrificed and the lungs, heart, liver, spleen and kidneys dissected. Luciferase activity was measured in the dissected tissues by bioluminescence imaging on an IVIS in vivo imaging system.

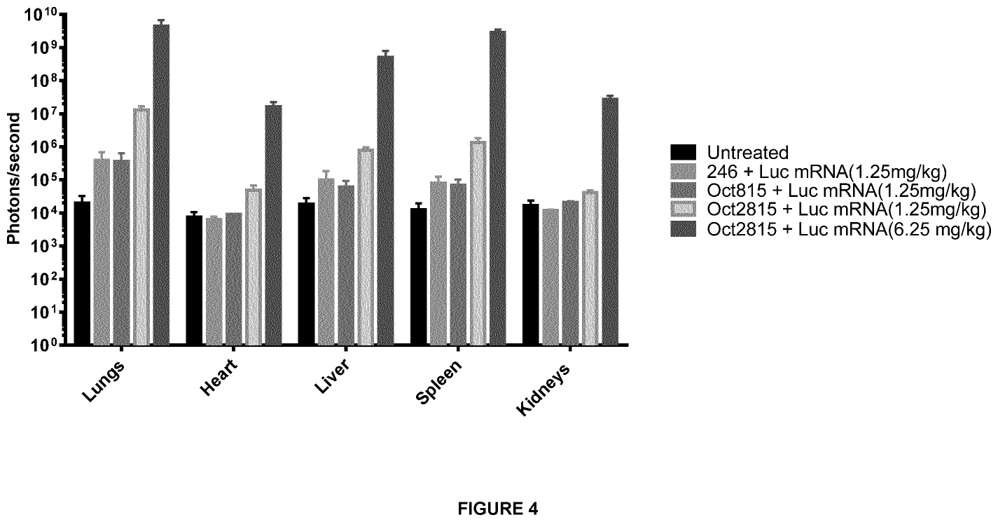

As shown in FIG. 4, all of the tested peptides mediated effective in vivo delivery of mRNA and luciferase protein expression in the various tissues upon systemic administration. Of the peptides tested, the most hydrophobic peptide (Oct2815) was shown to be the most effective.

Oct2815 was safely administered at very high dosing (6.25 mg/kg of mRNA/25 mg/kg of peptide) and induced high levels of protein expression from the delivered mRNA cargo without any apparent side-effects. The peptides of the invention are believed to be tolerated in a dose of up to 30 mg/kg in mice, which is at least 10-fold higher than the dose commonly known to be tolerated when using any CPP of the prior art in vivo.

### Example 5—Cas9 Protein Delivery

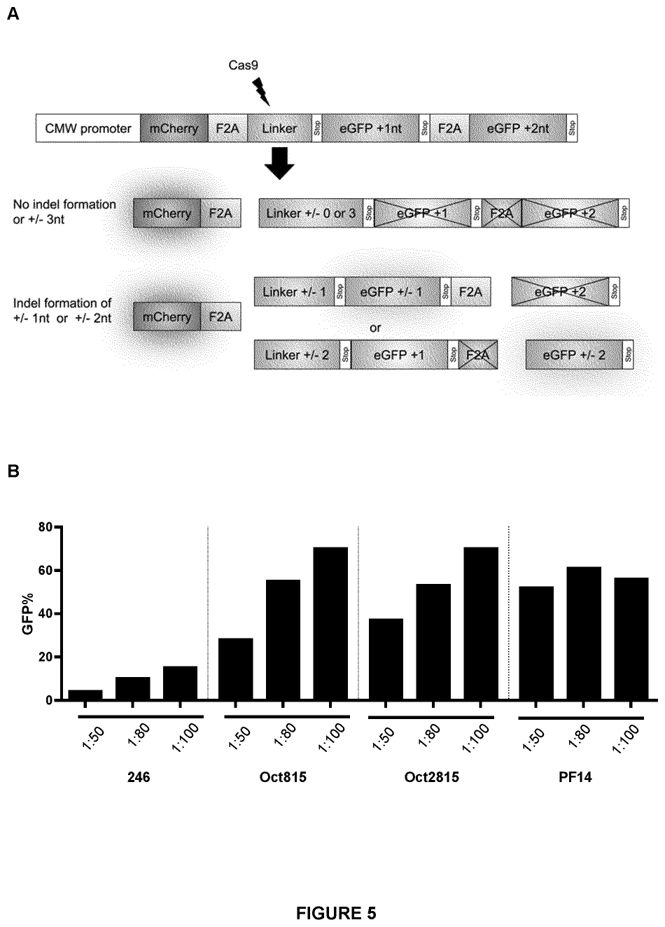

Delivery of proteins is another important modality for the peptides of the present invention. In order to investigate the ability of the peptides to deliver protein, a stop-light reporter system was employed. FIG. 5A illustrates the stop-light reporter system, which is a quick and simple system to evaluate indel formation in stably transduced cells. Cells are transfected with a construct containing an mCherry gene followed by two enhanced green fluorescent protein (eGFP) genes that are shifted out of frame by +1 and +2 nucleotides, respectively. The mCherry gene is constituently expressed, while the eGFP genes are only expressed if a double stranded break in the linker creates insertions or deletions of +/−1 or 2 bp. This occurs upon the effective delivery of Cas9 RNP system to the nucleus of cells, which leads to one of the two eGFP genes being in frame and thus expressed. Consequently, the induction of eGFP expression can be used to assess the efficacy of Cas9-mediated genome editing.

5k HEK293T Stop-Light cells were treated with 50 ng (0.31 μmol) Cas9 that was formulated with various peptides at different peptide-to-Cas9 RNP MRs (50:1-100:1). After 72 h of treatment, eGFP expression was analysed by fluorescence-activated cell sorting (FACS) analysis. Gene editing efficiency was calculated as a percentage of cells in the cell population where eGFP expression was induced by the treatment.

FIG. 5B shows the results of the experiment. As evidence by FIG. 5B, various peptides mediated efficient Cas9 RNP delivery and associated gene editing. The more hydrophobic analogues, such as Oct815 and Oct2815, were shown to be the most potent. FIG. 5B also shows the results as compared to a known CPP, PF14, and demonstrates that the peptides of the present invention are equally or more effective than the known CPPs.

### Example 6—Cas9 Protein Uptake and Transfection Efficiency Kinetics

To study the uptake and kinetics of peptides of the invention/Cas9 RNP complexes, an Atto550-labelled trans-activating CRISPR RNA (tracrRNA or trRNA) was utilised. For this, HEK wild-type (WT) cells were treated with peptide/Cas9 RNP complexes (100 ng Cas9, formulated at MR80). Cellular uptake and the kinetic profile were measured by FACS analysis by probing the fluorescence signal over different time points (0.25 to 6 h).

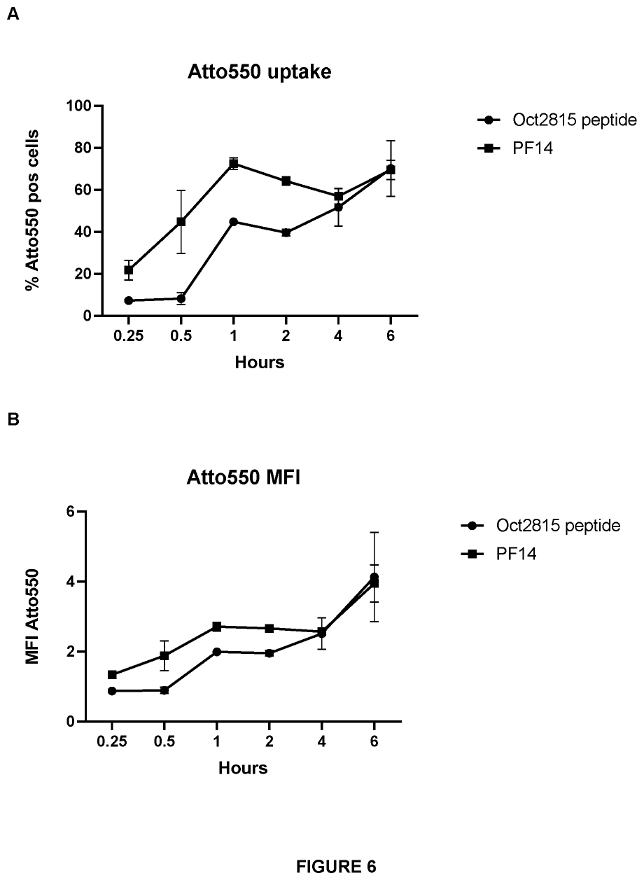

FIG. 6 demonstrates the speed and efficacy of uptake of Cas9 RNP into cells when complexed to Oct2815 as compared to a prior art CPP (PF14). In this regard, FIG. 6A shows that Oct2815/Cas9 RNP complexes are rapidly taken up by the cells and reach a major proportion of the cells within 1 h. FIG. 6B, showing mean fluorescence intensity (MFI), demonstrates that the transfection efficiency of the peptide/Cas9 RNP complexes in the cells was comparable to that of PF14. Both Oct2815 and the known CPP were taken up by the cells with high efficiency. Both peptides thus follow similar uptake kinetics and transfection efficiency.

### Example 7—Antibody Delivery by CPP

The ability of Oct2815 peptide to deliver antibodies to cells was also tested. In this experiment, HEK293 WT cells were treated with PE-conjugated IgG antibody that was formulated with Oct2815 peptide at MR50 (peptide:antibody). After treating the cells for 3 h at varying concentrations (0.5-4 nM), delivery efficiency was measured by FACS analysis by probing for the fluorescence of PE in the cells. In parallel, treatments were carried out with the naked PE-antibody, i.e. without the presence of the Oct2815 peptide.

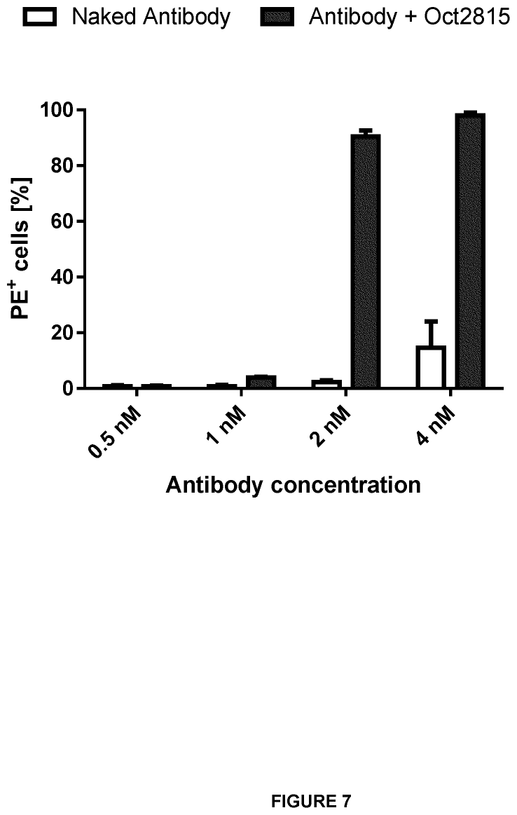

As shown in FIG. 7, Oct2815 mediated effective delivery of PE-conjugated antibodies into cells. It was significantly more effective in this regard than the naked PE-antibody.

### Example 8—Combined Cas9 RNP+mRNA Delivery

The ability of peptides of the invention to facilitate the simultaneous delivery and co-formulation compatibility for mRNA and Cas9 RNP was studied. In this study, Cas9 was combined with increasing amounts of luciferase-encoding mRNA and co-formulated with Oct2815 peptide at peptide-to-Cas9 molar ratio of 100. To study the co-delivery, HEK293T Stop-Light cells were treated with these formulations and the corresponding gene editing and luciferase values were measured by FACS (at 72 h) and luminometric analysis (at 24 h), respectively.

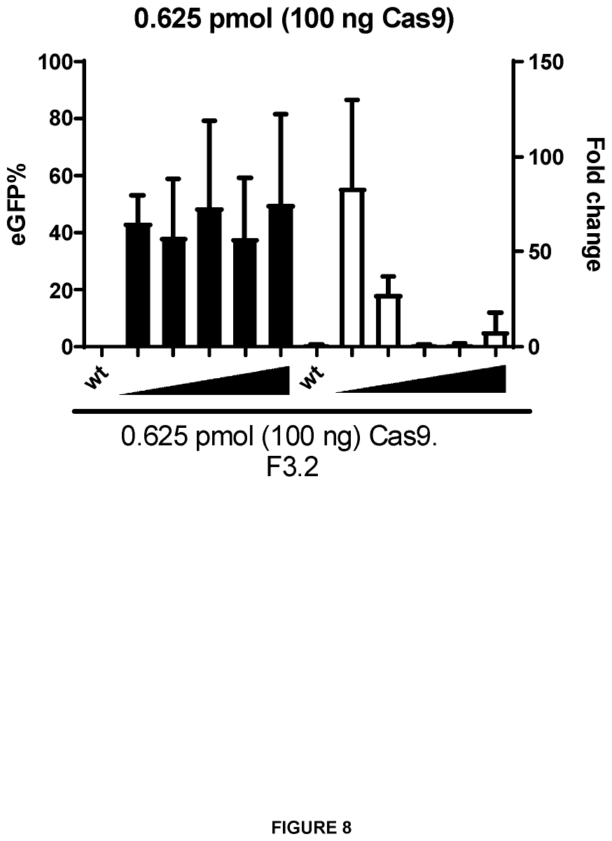

FIG. 8 illustrates the eGFP expression (black bars) and luciferase signal fold-change (white bars) of treated over the background of untreated cells.

As can be seen from FIG. 8, Oct2815 complexed with Cas9 RNP and mRNA enabled effective delivery and biological activity of both the Cas9 RNP gene editing system and protein-encoding mRNA simultaneously.

### Example 9—Vesicle Delivery Using CPP

To test the ability of Oct2815 to enhance the delivery of EVs, the peptide was mixed with WT EVs or EVs labelled with CD63-GFP or CD63-nLuc derived from HEK or MSC cells, respectively. WT and labelled EVs from both cell types, containing no peptide, were also included in the study as negative controls. The peptide/control-EVs were then administered to HEK cells. Uptake of EVs into cells was measured using GFP as the readout.

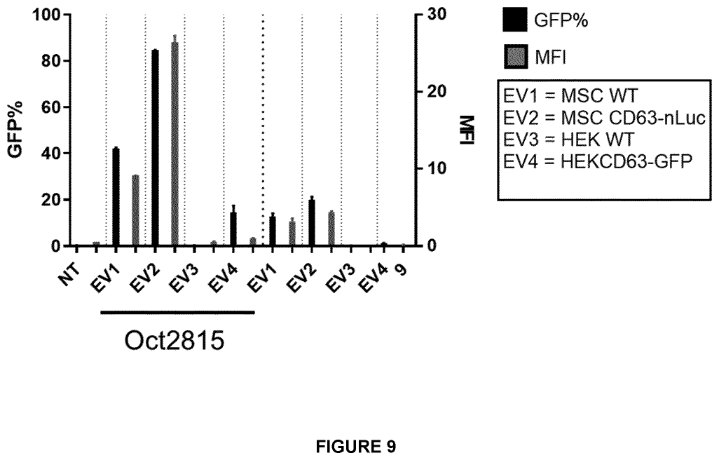

FIG. 9 shows that cells treated with peptide-EVs expressed significantly more GFP than those cells treated with control-EVs. This demonstrates that Oct2815 peptide significantly enhanced the transfection/delivery efficacy of various types of EVs from different EV cell sources.

### Example 10—Delivery of mRNA to the CNS

In order to investigate the ability of the peptide/nanoparticle to deliver cargos into brain/CNS, an in vivo experiment studying the delivery of mRNA directly to the CNS was designed.

Delivery of mRNA was tested using the Oct2815 peptides and a luciferase-encoding mRNA (Luc mRNA). Oct2815 peptide was formulated with 1 ug of mRNA dosing at a peptide-to-mRNA weight ratio 4 in 5 μl HEPES buffer+5% glucose. The formulation was subsequently injected by intracerebroventricular (ICV) injection into C57BL6/j female mice right ventricle (n=4). After 24 h of treatment, animals were injected with D-Luciferin (150 mg/kg) LP; after 10 minutes, mice were sacrificed and brain was dissected. Luciferase activity was measured ex vivo by bioluminescence imaging on an IVIS in vivo imaging system.

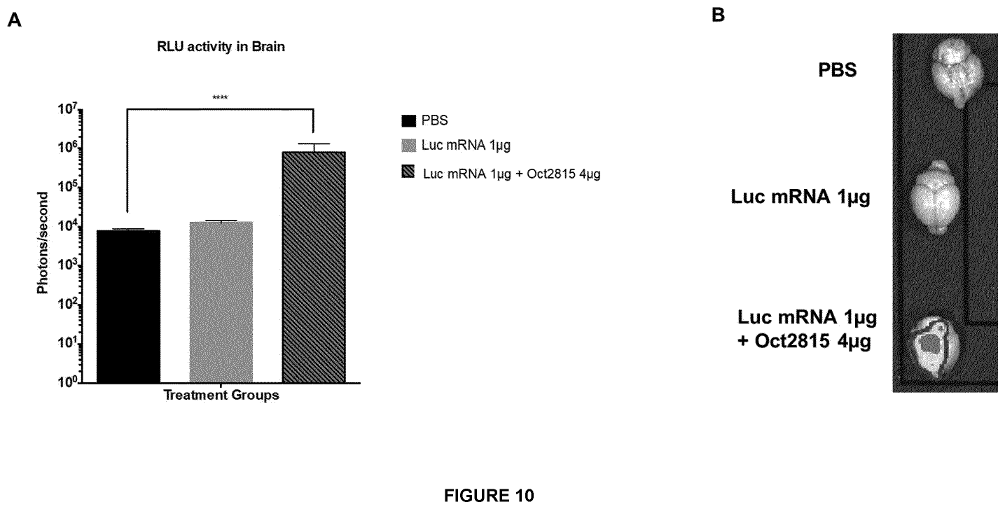

FIG. 10 shows that upon ICV administration, Oct2815 peptide mediated highly efficient mRNA delivery to the brain of mice and significantly enhanced the luciferase protein expression in the brain as compared to the injection of naked mRNA or vehicle (PBS). This demonstrates that peptides according to the invention are capable of transporting large cargos, such as mRNA molecules, to the brain upon administrating into cerebrospinal fluid (CSF).

### Example 11—Effect of Additives

In order to investigate the effect of additives on the efficacy of the peptides/nanoparticles of the invention a range of different additives were tested and the effect on delivery of Cas9 ribonuncleoprotein (RNP) was examined using the same stop-light reporter system described above and illustrated in FIG. 5.

Peptides Oct215 or Oct2815 were tested. Cas9 RNP was formulated in HBG buffer (HEPES buffered glucose) with either glycerol, PEG, PVP10, PVP55, PVA18, PVA40 or PVA-PEG. Oct215 or 2815 was added and incubated at room temperature for 40 min to allow peptide nanoparticles to form (1.1:1 sgRNA to Cas9 and 1:150 RNP:CPP).

Following incubation HEK293T Stop-Light cells were treated with the peptide RNP complex (25 ng Cas9 added to each well). After 72 h of treatment, eGFP expression was analysed by fluorescence-activated cell sorting (FACS) analysis. Gene editing efficiency was calculated as the percentage of eGFP expressing cells in the viable mCherry expressing population, mCherry being the marker for reporter construct expression and eGFP for gene editing of said construct.

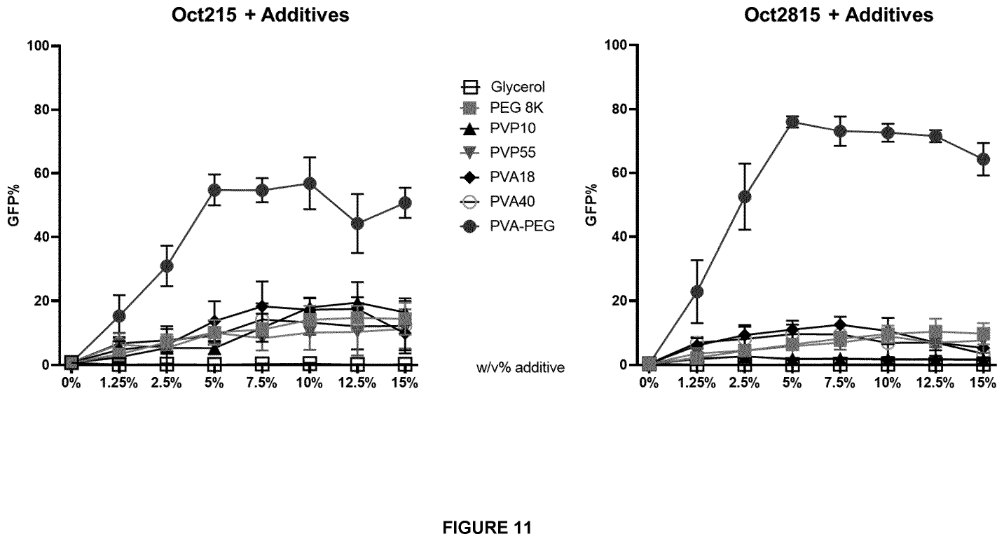

FIG. 11 shows the effect of different additives on the efficiency of gene editing by Cas9. Most additives improved productive delivery, raising editing rates from undetectable levels to between 10-20%. Notably PVA-PEG dramatically improved the efficacy of cargo delivery up to 60% for Oct215 and up to 80% for Oct2815.

### Example 12—siRNA Delivery and Effect of Additives on siRNA Delivery

To investigate the ability of the peptides/nanoparticles to deliver siRNAs into cells an in vitro study in the luciferase stable HEK293 reporter cell line was designed. Oct2815 peptide was mixed with a luciferase-targeting siRNA or control (Ctrl) siRNA at a peptide-to-siRNA molar ratio of 30 (MR30) in the HBG buffer (HEPES-buffered glucose) and incubated at RT for 30 min to allow peptide nanoparticles formation. For the inclusion of the additives, peptide/siRNA nanoparticles were further mixed and incubated with MilliQ (MQ) water, Hepes pH7.4, HBG, Dulbecco's phosphate buffered saline (DPBS), NaCl, sucrose, PVP40 & PVA-PEG before they were added to the cells.

After the incubation, luciferase stable HEK293 cells (HEK293_Luc) cells were treated with the peptide/siRNA formulations at different siRNA concentrations ranging from 200-12 nM. After 24 h of treatment, luciferase expression was analysed by luminometeric analysis. Gene knockdown efficiency was calculated as the percentage of luciferase expression in untreated cells.

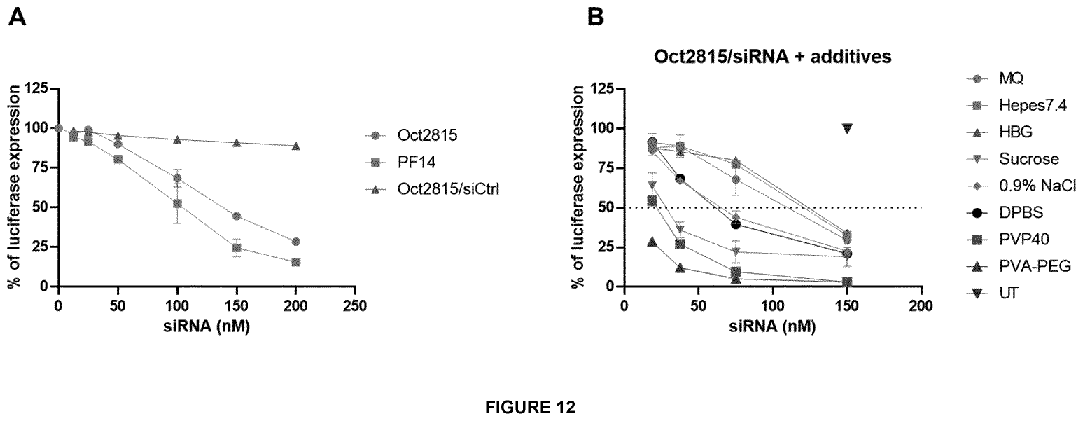

FIG. 12A shows the ability of Oct2815 peptide to mediate effective siRNA delivery. Treatment of HEK293_Luc cells with Oct2815/siRNA nanoparticles induced effective dose-dependent target gene silencing as measured by decreased luciferase activity, while Oct2815 nanoparticles with control (Ctrl) siRNA did not affect the luciferase levels. As compared to the control CPP (PF14), gene silencing activity of Oct2815 peptide remained in the same range. FIG. 12B shows the effect of various additives in enhancing the activity of Oct2815/siRNA nanoparticles. Notably sucrose, PVP40 and PVA-PEG all dramatically further improved the delivery efficacy and gene silencing activity of Oct2815/siRNA nanoparticles.

1. A peptide comprising a first sequence A1-B1-C1-(D1)ₙ and a second sequence A2-B2-C2-(D2)ₘ, wherein:
A1 is an amino acid that has a positively charged side chain at pH 7;
B1 is an amino acid having a hydrophobic side chain;
C1 is an amino acid having a hydrophobic side chain;
D1, when present, is an amino acid having a hydrophobic side chain; and
n is 0 or 1;
wherein one of B1, C1, or D1 is substituted at the α carbon with a terminal alkenyl group or terminal alkynyl group; and
A2 is an amino acid that has a positively charged side chain at pH 7;
B2 is an amino acid having a hydrophobic side chain;
C2 is an amino acid having a hydrophobic side chain;
D2, when present, is an amino acid having a hydrophobic side chain; and
m is 0 or 1;
wherein at least one of B2, C2, or D2 is substituted at the α carbon with a terminal alkenyl group or terminal alkynyl group.

2. (canceled)

3. (canceled)

4. A peptide as claimed in claim 1, wherein n is 1 and D1 is unsubstituted.

5. (canceled)

6. (canceled)

7. A peptide as claimed in claim 1, wherein m is 1 and D2 is unsubstituted.

8. A peptide as claimed in claim 1, wherein the amino acid of the first sequence that is substituted with a terminal alkenyl or terminal alkynyl group is located at position i, and the amino acid in the second sequence that is substituted with a terminal alkenyl or terminal alkynyl group is located at i+3, 4, 5, 6, 7, 8, 9, 10, 11, 12, 13, 14, 15, or 16.

9. A peptide as claimed in claim 1, further comprising a third sequence A3-B3-(C3)ₚ that is optionally conjugated to the C-terminus of the first sequence, wherein:
A3 is an amino acid that has a positively charged side chain at pH 7;
B3 is an amino acid having a hydrophobic side chain;
C3, when present, is an amino acid having a hydrophobic side chain; and
p is 0 or 1;
wherein B3 and C3 are independently (i) unsubstituted or (ii) substituted at the α carbon with a terminal alkenyl group or terminal alkynyl group.

10. (canceled)

11. A peptide as claimed in claim 9, wherein p is 1 and C3 is unsubstituted.

12. (canceled)

13. (canceled)

14. A peptide as claimed in claim 1, further comprising a fourth sequence A4-B4-(C4)_{q}, that is optionally conjugated to the N-terminus of the first sequence, wherein:
A4 is an amino acid that has a positively charged side chain at pH 7;
B4 is an amino acid having a hydrophobic side chain;
C4, when present, is an amino acid having a hydrophobic side chain; and
q is 0 or 1;
wherein B4 and C4 are independently (i) unsubstituted or (ii) substituted at the α carbon with a terminal alkenyl group or terminal alkynyl group.

15. (canceled)

16. (canceled)

17. A peptide as claimed in claim 14, wherein q is 1 and C4 is unsubstituted.

18. A peptide as claimed in claim 14, further comprising a fifth sequence B5-C5-(D5)ᵣ, that is optionally conjugated to the N-terminus of the fourth sequence, wherein:
B5 is an amino acid having a hydrophobic side chain;
C5 is an amino acid having a hydrophobic side chain;
D5, when present, is an amino acid having a hydrophobic side chain; and
r is 0 or 1;
wherein B5 and C5 are independently (i) unsubstituted or (ii) substituted at the α carbon with a terminal alkenyl group or terminal alkynyl group.

19. A peptide as claimed in claim 18, wherein the fifth sequence is conjugated to the N-terminus of the fourth sequence.

20.-23. (canceled)

24. A peptide as claimed in claim 1, further comprising a sixth sequence A6-B6-C6-(D6)ₛ, that is optionally conjugated to the C-terminus of the second sequence, wherein:
A6 is an amino acid that has a positively charged side chain at pH 7;
B6 is an amino acid having a hydrophobic side chain;
C6 is an amino acid having a hydrophobic side chain;
D6, when present, is an amino acid having a hydrophobic side chain; and
s is 0 or 1;
wherein B6 and C6 are independently (i) unsubstituted or (ii) substituted at the α carbon with a terminal alkenyl group or terminal alkynyl group.

25.-28. (canceled)

29. A peptide as claimed in claim 1, wherein the peptide has the following sequence:
B5-C5-(D5)ᵣ-A4-B4-(C4)_{q}-A1-B1-C1-(D1)ₙ-A3-B3-(C3)ₚ-A2-B2-C2-(D2)ₘ-A6-B6-C6-(D6)ₛ, wherein:
A1 is an amino acid that has a positively charged side chain at pH 7;
B1 is an amino acid having a hydrophobic side chain;
C1 is an amino acid having a hydrophobic side chain;
D1, when present, is an amino acid having a hydrophobic side chain; and
n is 0 or 1;
wherein one of B1, C1, or D1 is substituted at the α carbon with a terminal alkenyl group or terminal alkynyl group;
A2 is an amino acid that has a positively charged side chain at pH 7;
B2 is an amino acid having a hydrophobic side chain;
C2 is an amino acid having a hydrophobic side chain;
D2, when present, is an amino acid having a hydrophobic side chain; and
m is 0 or 1;
wherein at least one of B2, C2, or D2 is substituted at the α carbon with a terminal alkenyl group or terminal alkynyl group;
A3 is an amino acid that has a positively charged side chain at pH 7;
B3 is an amino acid having a hydrophobic side chain;
C3, when present, is an amino acid having a hydrophobic side chain; and
p is 0 or 1;
wherein B3 and C3 are independently (i) unsubstituted or (ii) substituted at the α carbon with a terminal alkenyl group or terminal alkynyl group;
A4 is an amino acid that has a positively charged side chain at pH 7;
B4 is an amino acid having a hydrophobic side chain;
C4, when present, is an amino acid having a hydrophobic side chain; and
q is 0 or 1;
wherein B4 and C4 are independently (i) unsubstituted or (ii) substituted at the α carbon with a terminal alkenyl group or terminal alkynyl group;
B5 is an amino acid having a hydrophobic side chain;
C5 is an amino acid having a hydrophobic side chain;
D5, when present, is an amino acid having a hydrophobic side chain; and
r is 0 or 1;
wherein B5 and C5 are independently (i) unsubstituted or (ii) substituted at the α carbon with a terminal alkenyl group or terminal alkynyl group; and
A6 is an amino acid that has a positively charged side chain at pH 7;
B6 is an amino acid having a hydrophobic side chain;
C6 is an amino acid having a hydrophobic side chain;
D6, when present, is an amino acid having a hydrophobic side chain; and
s is 0 or 1;
wherein B6 and C6 are independently (i) unsubstituted or (ii) substituted at the α carbon with a terminal alkenyl group or terminal alkynyl group.

30. A peptide as claimed in claim 1, further comprising a seventh sequence [A7-B7-C7-(D7)ₜ]ᵤ, and/or an eighth sequence [A8-B8-C8-(D8)ₓ]_{y}, wherein:
A7 is an amino acid that has a positively charged side chain at pH 7;
B7 is an amino acid having a hydrophobic side chain;
C7 is an amino acid having a hydrophobic side chain;
D7, when present, is an amino acid having a hydrophobic side chain;
t is 0 or 1; and
u is 0 to 6;
A8 is an amino acid that has a positively charged side chain at pH 7;
B8 is an amino acid having a hydrophobic side chain;
C8 is an amino acid having a hydrophobic side chain;
D8, when present, is an amino acid having a hydrophobic side chain;
x is 0 or 1; and
y is 0 to 6.

31. A peptide as claimed in claim 1, wherein the peptide has the following sequence:
[A7-B7-C7-(D7)ₜ]ᵤ-B5-C5-(D5)ᵣ-A4-B4-(C4)_{q}-A1-B1-C1-(D1)ₙ-A3-B3-(C3)ₚ-A2-B2-C2-(D2)ₘ-A6-B6-C6(D6)ₛ-[A8-B8-C8-(D8)ₓ]_{y},
wherein:
A1 is an amino acid that has a positively charged side chain at pH 7;
B1 is an amino acid having a hydrophobic side chain;
C1 is an amino acid having a hydrophobic side chain;
D1, when present, is an amino acid having a hydrophobic side chain; and
n is 0 or 1;
wherein one of B1, C1, or D1 is substituted at the α carbon with a terminal alkenyl group or terminal alkynyl group;
A2 is an amino acid that has a positively charged side chain at pH 7;
B2 is an amino acid having a hydrophobic side chain;
C2 is an amino acid having a hydrophobic side chain;
D2, when present, is an amino acid having a hydrophobic side chain; and
m is 0 or 1;
wherein at least one of B2, C2, or D2 is substituted at the α carbon with a terminal alkenyl group or terminal alkynyl group;
A3 is an amino acid that has a positively charged side chain at pH 7;
B3 is an amino acid having a hydrophobic side chain;
C3, when present, is an amino acid having a hydrophobic side chain; and
p is 0 or 1;
wherein B3 and C3 are independently unsubstituted or substituted at the α carbon with a terminal alkenyl group or terminal alkynyl group;
A4 is an amino acid that has a positively charged side chain at pH 7;
B4 is an amino acid having a hydrophobic side chain;
C4, when present, is an amino acid having a hydrophobic side chain; and
q is 0 or 1;
wherein B4 and C4 are independently (i) unsubstituted or (ii) substituted at the α carbon with a terminal alkenyl group or terminal alkynyl group;
B5 is an amino acid having a hydrophobic side chain;
C5 is an amino acid having a hydrophobic side chain;
D5, when present, is an amino acid having a hydrophobic side chain; and
r is 0 or 1;
wherein B5 and C5 are independently (i) unsubstituted or (ii) substituted at the α carbon with a terminal alkenyl group or terminal alkynyl group;
A6 is an amino acid that has a positively charged side chain at pH 7;
B6 is an amino acid having a hydrophobic side chain;
C6 is an amino acid having a hydrophobic side chain;
D6, when present, is an amino acid having a hydrophobic side chain; and
s is 0 or 1;
wherein B6 and C6 are independently unsubstituted or substituted at the α carbon with a terminal alkenyl group or terminal alkynyl group;
A7 is an amino acid that has a positively charged side chain at pH 7;
B7 is an amino acid having a hydrophobic side chain;
C7 is an amino acid having a hydrophobic side chain;
D7, when present, is an amino acid having a hydrophobic side chain;
t is 0 or 1; and
u is 0 to 6; and
A8 is an amino acid that has a positively charged side chain at pH 7;
B8 is an amino acid having a hydrophobic side chain;
C8 is an amino acid having a hydrophobic side chain; and
D8, when present, is an amino acid having a hydrophobic side chain.

32. A peptide as claimed in claim 1, wherein each terminal alkenyl group or terminal alkynyl group comprises at least 5 carbon atoms, optionally from 5 to carbon atoms, optionally from 5 to 15 carbon atoms, optionally from 5 to 11 carbon atoms, optionally from 5 to 10 carbon atoms, optionally from 5 to 8 carbon atoms.

33. A peptide as claimed in claim 1, wherein each alkenyl or alkynyl group is independently selected from 4-pentenyl, 5-hexenyl, 6-heptenyl, 7-octenyl, 8-nonenyl and 9-decenyl, 4-pentynyl, 5-hexynyl, 6-heptynyl, 7-octynyl, 8-nonynyl and 9-decynyl, optionally wherein each alkenyl group is independently selected from 4-pentenyl and 7-octenyl.

34. A peptide as claimed in claim 1, wherein the alkenyl or alkynyl groups in combination comprise at least 11 carbon atoms, optionally at least 13 carbon atoms.

35. A peptide as claimed in claim 1, wherein the peptide has an essentially alpha helical conformation as determined by helical wheel projection, optionally wherein the peptide has at least 90% alpha helicity, optionally at least 99% alpha helicity.

36. A peptide as claimed in claim 1, wherein each amino acid that has a positively charged side chain at pH 7 is individually selected from the group consisting of arginine, ornithine, histidine and lysine, and analogues thereof, optionally individually selected from the group consisting of arginine, ornithine and lysine, optionally wherein each amino acid that has a positively charged side chain at pH 7 is lysine or arginine.

37. A peptide as claimed in claim 1, wherein each amino acid having a hydrophobic side chain is individually selected from the group consisting of glycine, valine, alanine, isoleucine, leucine, proline and methionine, optionally wherein each amino acid having a hydrophobic side chain is individually selected from the group consisting of leucine and alanine.

38. (canceled)

39. A peptide as claimed in claim 1, wherein the peptide is bound covalently or non-covalently to a cargo molecule.

40. A peptide as claimed in claim 39, wherein the cargo molecule is or comprises at least one biomacromolecule, optionally a nanoparticle, optionally an extracellular vesicle (EV), optionally an exosome, a microvesicle or an ARRDC1-mediated microvesicle (ARRM), or a virus, optionally a lentivirus, an adenovirus, an adeno-associated virus (AAV), a retrovirus, a respiratory syncytial virus (RSV), a herpes simplex virus (HSV), an encapsulated virus, a non-encapsulated virus or a naked viral genome; a protein, a polypeptide, a peptide; an oligonucleotide and/or a polynucleotide, optionally an antisense oligonucleotide, a splice-switching oligonucleotide, a short interfering RNA (siRNA), a short activating RNA (saRNA), a short hairpin RNA (shRNA), a microRNA (miRNA), an anti-miRNA, a plasmid DNA, a mini-circle, an mRNA, a viral genome and/or viral genetic material; an RNP such as CRISPR-Cas; or a gene editing technology such as a TALEN, a Zinc finger or CRISPR-Cas.

41. A pharmaceutical composition comprising a peptide as claimed in claim 1 and a pharmaceutically acceptable carrier, optionally wherein the pharmaceutically acceptable carrier comprises an aqueous solution, optionally an aqueous solution comprising 4-(2-hydroxyethyl)-1-piperazineethanesulfonic acid (HEPES), wherein the pharmaceutically acceptable carrier optionally comprises a monosaccharide, disaccharide, polyvinylpyrrolidone, polyvinyl alcohol, dihydric alcohol, polyhydric alcohol (optionally sorbitol, polyethylene glycol or propylene glycol) and/or a detergent, optionally a polyoxyethylenesorbitan (Tween).
